SEQUENCE LISTING (113 sequences)

SEQ ID NO: 1 - DNA, 720 bp, Artificial Sequence
  Description of Artificial Sequence Synthetic polynucleotide
atggtgtccaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtaccaaggg

SEQ ID NO: 2 - DNA, 2435 bp, Artificial Sequence
  Description of Artificial Sequence Synthetic polynucleotide
gggttattgtctcatgagcggatacatatttgaatgtatttagaaaaataaacaaataggggttccgcgcacatttccccgaaaagtgccacgtctccaatgagaagagcctgcagcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctggagctccaccgcggtggcggccgctctagaactagtggatcccccgggctgcaggaattcgatatcaagcttatcgataccgtcgacctcgagggggggcccggtacccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacctcgaggctcttctgggaggagacgatccaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcaccaagcttcagaagaactcgtcaagaaggcgatagaaggcgatgcgctgcgaatcgggagcggcgataccgtaaagcacgaggaagcggtcagcccattcgccgccaagctcctcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgttcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcgaagcatttatca

SEQ ID NO: 3 - DNA, 2457 bp, Artificial Sequence
  Description of Artificial Sequence Synthetic polynucleotide
gggttattgtctcatgagcggatacatatttgaatgtatttagaaaaataaacaaataggggttccgcgcacatttccccgaaaagtgccacgtctccaatggtgtccaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtaccaagggaggagacgatccaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcaccaagcttcagaagaactcgtcaagaaggcgatagaaggcgatgcgctgcgaatcgggagcggcgataccgtaaagcacgaggaagcggtcagcccattcgccgccaagctcctcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgttcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcgaagcatttatca

SEQ ID NO: 4 - DNA, 2481 bp, Artificial Sequence
  Description of Artificial Sequence Synthetic polynucleotide
aatgtccggaggtggcggtgggagcctggaagttctgttccaggggccaatgagagacgctgcagcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctggagctccaccgcggtggcggccgctctagaactagtggatcccccgggctgcaggaattcgatatcaagcttatcgataccgtcgacctcgagggggggcccggtacccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacctcgagcgtctcagggagaagagcatccaaaggcggtaatacggttatccgcggaacccctatttgtttatttttctaaatacattcaaatatgtatccgctcatgagacaataaccctgataaatgcttcgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagaacatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaggagcttggcggcgaatgggctgaccgcttcctcgtgctttacggtatcgccgctcccgattcgcagcgcatcgccttctatcgccttcttgacgagttcttctgaagcttggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtgcacatttccccgaaaagtgccagctcttc

SEQ ID NO: 5 - DNA, 1861 bp, Artificial Sequence
  Description of Artificial Sequence Synthetic polynucleotide
caatgagaagagcaagcttgctcttctgggaggagaccatccaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcaccaagcttgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccaggtctc

SEQ ID NO: 6 - DNA, 2577 bp, Artificial Sequence
  Description of Artificial Sequence Synthetic polynucleotide
caatgtccggaggtggcggtgggagcctggaagttctgttccaggggccaatgagagacgctgcagcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctggagctccaccgcggtggcggccgctctagaactagtggatcccccgggctgcaggaattcgatatcaagcttatcgataccgtcgacctcgagggggggcccggtacccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacctcgagcgtctcagggaggagaccatccaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcaccaagcttgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccaggtctc

SEQ ID NO: 7 - DNA, 2529 bp, Artificial Sequence
  Description of Artificial Sequence Synthetic polynucleotide
caatgagagacgctgcagcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctggagctccaccgcggtggcggccgctctagaactagtggatcccccgggctgcaggaattcgatatcaagcttatcgataccgtcgacctcgagggggggcccggtacccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacctcgagcgtctcagggaggagaccatccaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcaccaagcttgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccaggtctc

SEQ ID NO: 8 - DNA, 1785 bp, Artificial Sequence
  Description of Artificial Sequence Synthetic polynucleotide
caatgagagaccctaatcaaaagcttctaatcaaggtctctgggagctaaggaagagcatccaaaggcggtaatacggttatccgcggaacccctatttgtttatttttctaaatacattcaaatatgtatccgctcatgagacaataaccctgataaatgcttcgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagaacatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaggagcttggcggcgaatgggctgaccgcttcctcgtgctttacggtatcgccgctcccgattcgcagcgcatcgccttctatcgccttcttgacgagttcttctgaagcttggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtgcacatttccccgaaaagtgccagctctt

SEQ ID NO: 9 - DNA, 2439 bp, Artificial Sequence
  Description of Artificial Sequence Synthetic polynucleotide
caatgagagacgctgcagcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctggagctccaccgcggtggcggccgctctagaactagtggatcccccgggctgcaggaattcgatatcaagcttatcgataccgtcgacctcgagggggggcccggtacccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacctcgagcgtctcagggagctaaggaagagcatccaaaggcggtaatacggttatccgcggaacccctatttgtttatttttctaaatacattcaaatatgtatccgctcatgagacaataaccctgataaatgcttcgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagaacatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaggagcttggcggcgaatgggctgaccgcttcctcgtgctttacggtatcgccgctcccgattcgcagcgcatcgccttctatcgccttcttgacgagttcttctgaagcttggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtgcacatttccccgaaaagtgccagctctt

SEQ ID NO: 10 - DNA, 2931 bp, Artificial Sequence
  Description of Artificial Sequence Synthetic polynucleotide
caatgaaaagacccttggaaggtattcgtgtacttgatttaacacaggcttacagtggccccttttgtacaatgaatcttgctgatcatggtgctgaggttattaaaattgagcgccccggcagtggagatcaaacaagaggttgggggcctatggaaaatgactacagtggctactatgcttacattaaccgtaataaaaaaggaatcaccttaaatcttgcttccgaagaaggaaagaaagtttttgccgaattggttaaatctgccgatgtgatttgcgaaaactataaggttggtgttttagaaaaattaggcttttcctatgaggtcttaaaagaactcaacccccgcatcatttatggctccatcagcggttttggattaacaggtgaattgtcctcccgcccctgctatgatatcgtcgctcaagcaatgagcggaatgatgagtgtaaccggctttgcagacggtcctccctgcaaaatcggcccttctgtaggagatagctatactggtgcatatttgtgcatgggtgttttgatggcattatacgaaagagaaaaaacaggcgttggccgccgtatcgatgtgggaatggtagataccctgttctctacaatggaaaactttgttgttgaatacaccattgctggtaagcatccccaccgtgcaggcaatcaagatccaagtattgccccttttgactcctttagggcaaaagattcggattttgtaatggggtgtggcacaaacaaaatgtttgcaggactatgtaaagcaatgggcagagaggatttgattgatgatcctcgtttcaatacaaacctgaatcgttgtgataactatttaaatgacttaaagccaatcatcgaagaatggacccaaacaaagaccgttgcagagttagaggaaatcatctgcggactttccattcccttcggcccaatcctcacgattcccgagatttctgagcattccttaacaaaagaaagaaatatgctttgggaagtttatcagcctggcatggatagaacaattcgcattcccggctcccctattaaaatccacggtgaagaagataaggctcagaaaggtgcccctattctgggagaagacaattttgctgtctacgcagaaattttaggtctctcagtagaagaaattaaatcactggaagagaaaaatgtcatcgggaggagacgatccaaaggcggtaatacggttatccgcggaacccctatttgtttatttttctaaatacattcaaatatgtatccgctcatgagacaataaccctgataaatgcttcgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagaacatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaggagcttggcggcgaatgggctgaccgcttcctcgtgctttacggtatcgccgctcccgattcgcagcgcatcgccttctatcgccttcttgacgagttcttctgaagcttggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtgcacatttccccgaaaagtgccacgtctc

SEQ ID NO: 11 - DNA, 4003 bp, Artificial Sequence
  Description of Artificial Sequence Synthetic polynucleotide
aaatgggagacgggatcccccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctccaccgcggtggcggccgctctagaactagtggatcccccgggctgcaggaattcgatatcaagcttatcgataccgtcgacctcgagggggggcccggtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacggatcccgtctcggggagcagaggatcgcatcaccatcaccatcactaataagcttgacctgtgaagtgaaaaatggcgcacattgtgcgacattttttttgtctgccgtttaccgctactgcgtcacggatctccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattgatagactggatggaggcggataaagttgcaggaccacttctgcgctcggcccttccggctggctggtttattgctgataaatctggagccggtgagcgtggctctcgcggtatcattgcagcactggggccagatggtaagccctcccgtatcgtagttatctacacgacggggagtcaggcaactatggatgaacgaaatagacagatcgctgagataggtgcctcactgattaagcattggtaggaattaatgatgtctcgtttagataaaagtaaagtgattaacagcgcattagagctgcttaatgaggtcggaatcgaaggtttaacaacccgtaaactcgcccagaagctaggtgtagagcagcctacattgtattggcatgtaaaaaataagcgggctttgctcgacgccttagccattgagatgttagataggcaccatactcacttttgccctttagaaggggaaagctggcaagattttttacgtaataacgctaaaagttttagatgtgctttactaagtcatcgcgatggagcaaaagtacatttaggtacacggcctacagaaaaacagtatgaaactctcgaaaatcaattagcctttttatgccaacaaggtttttcactagagaatgcattatatgcactcagcgcagtggggcattttactttaggttgcgtattggaagatcaagagcatcaagtcgctaaagaagaaagggaaacacctactactgatagtatgccgccattattacgacaagctatcgaattatttgatcaccaaggtgcagagccagccttcttattcggccttgaattgatcatatgcggattagaaaaacaacttaaatgtgaaagtgggtcttaaaagcagcataacctttttccgtgatggtaacttcactagtttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgacccgacaccatcgaatggccagatgattaattcctaatttttgttgacactctatcattgatagagttattttaccactccctatcagtgatagagaaaagtgaaatgaatagttcgacaaaaattctagaaataattttgtttaactttaagaaggagatatac

SEQ ID NO: 12 - DNA, 4363 bp, Artificial Sequence
  Description of Artificial Sequence Synthetic polynucleotide
tctagaaataattttgtttaactttaagaaggagatatacaaatgaaaagacccttggaaggtattcgtgtacttgatttaacacaggcttacagtggccccttttgtacaatgaatcttgctgatcatggtgctgaggttattaaaattgagcgccccggcagtggagatcaaacaagaggttgggggcctatggaaaatgactacagtggctactatgcttacattaaccgtaataaaaaaggaatcaccttaaatcttgcttccgaagaaggaaagaaagtttttgccgaattggttaaatctgccgatgtgatttgcgaaaactataaggttggtgttttagaaaaattaggcttttcctatgaggtcttaaaagaactcaacccccgcatcatttatggctccatcagcggttttggattaacaggtgaattgtcctcccgcccctgctatgatatcgtcgctcaagcaatgagcggaatgatgagtgtaaccggctttgcagacggtcctccctgcaaaatcggcccttctgtaggagatagctatactggtgcatatttgtgcatgggtgttttgatggcattatacgaaagagaaaaaacaggcgttggccgccgtatcgatgtgggaatggtagataccctgttctctacaatggaaaactttgttgttgaatacaccattgctggtaagcatccccaccgtgcaggcaatcaagatccaagtattgccccttttgactcctttagggcaaaagattcggattttgtaatggggtgtggcacaaacaaaatgtttgcaggactatgtaaagcaatgggcagagaggatttgattgatgatcctcgtttcaatacaaacctgaatcgttgtgataactatttaaatgacttaaagccaatcatcgaagaatggacccaaacaaagaccgttgcagagttagaggaaatcatctgcggactttccattcccttcggcccaatcctcacgattcccgagatttctgagcattccttaacaaaagaaagaaatatgctttgggaagtttatcagcctggcatggatagaacaattcgcattcccggctcccctattaaaatccacggtgaagaagataaggctcagaaaggtgcccctattctgggagaagacaattttgctgtctacgcagaaattttaggtctctcagtagaagaaattaaatcactggaagagaaaaatgtcatcgggagcagaggatcgcatcaccatcaccatcactaataagcttgacctgtgaagtgaaaaatggcgcacattgtgcgacattttttttgtctgccgtttaccgctactgcgtcacggatctccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattgatagactggatggaggcggataaagttgcaggaccacttctgcgctcggcccttccggctggctggtttattgctgataaatctggagccggtgagcgtggctctcgcggtatcattgcagcactggggccagatggtaagccctcccgtatcgtagttatctacacgacggggagtcaggcaactatggatgaacgaaatagacagatcgctgagataggtgcctcactgattaagcattggtaggaattaatgatgtctcgtttagataaaagtaaagtgattaacagcgcattagagctgcttaatgaggtcggaatcgaaggtttaacaacccgtaaactcgcccagaagctaggtgtagagcagcctacattgtattggcatgtaaaaaataagcgggctttgctcgacgccttagccattgagatgttagataggcaccatactcacttttgccctttagaaggggaaagctggcaagattttttacgtaataacgctaaaagttttagatgtgctttactaagtcatcgcgatggagcaaaagtacatttaggtacacggcctacagaaaaacagtatgaaactctcgaaaatcaattagcctttttatgccaacaaggtttttcactagagaatgcattatatgcactcagcgcagtggggcattttactttaggttgcgtattggaagatcaagagcatcaagtcgctaaagaagaaagggaaacacctactactgatagtatgccgccattattacgacaagctatcgaattatttgatcaccaaggtgcagagccagccttcttattcggccttgaattgatcatatgcggattagaaaaacaacttaaatgtgaaagtgggtcttaaaagcagcataacctttttccgtgatggtaacttcactagtttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgacccgacaccatcgaatggccagatgattaattcctaatttttgttgacactctatcattgatagagttattttaccactccctatcagtgatagagaaaagtgaaatgaatagttcgacaaaaa

SEQ ID NO: 13 - DNA, 3153 bp, Artificial Sequence
  Description of Artificial Sequence Synthetic polynucleotide
gggttattgtctcatgagcggatacatatttgaatgtatttagaaaaataaacaaataggggttccgcgcacatttccccgaaaagtgccacgtctccaatgaaacaaagcactattgcactggcactcttaccgttactgtttacccctgtgacaaaagcccggacaccagaaatgcctgttctggaaaaccgggctgctcagggcgatattactgcacccggcggtgctcgccgtttaacgggtgatcagactgccgctctgcgtgattctcttagcgataaacctgcaaaaaatattattttgctgattggcgatgggatgggggactcggaaattactgccgcacgtaattatgccgaaggtgcgggcggcttttttaaaggtatagatgccttaccgcttaccgggcaatacactcactatgcgctgaataaaaaaaccggcaaaccggactacgtcaccgactcggctgcatcagcaaccgcctggtcaaccggtgtcaaaacctataacggcgcgctgggcgtcgatattcacgaaaaagatcacccaacgattctggaaatggcaaaagccgcaggtctggcgaccggtaacgtttctaccgcagagttgcaggatgccacgcccgctgcgctggtggcacatgtgacctcgcgcaaatgctacggtccgagcgcgaccagtgaaaaatgtccgggtaacgctctggaaaaaggcggaaaaggatcgattaccgaacagctgcttaacgctcgtgccgacgttacgcttggcggcggcgcaaaaacctttgctgaaacggcaaccgctggtgaatggcagggaaaaacgctgcgtgaacaggcacaggcgcgtggttatcagttggtgagcgatgctgcctcactgaattcggtgacggaagcgaatcagcaaaaacccctgcttggcctgtttgctgacggcaatatgccagtgcgctggctaggaccgaaagcaacgtaccatggcaatatcgataagcccgcagtcacctgtacgccaaatccgcaacgtaatgacagtgtaccaaccctggcgcagatgaccgacaaagccattgaattgttgagtaaaaatgagaaaggctttttcctgcaagttgaaggtgcgtcaatcgataaacaggatcatgctgcgaatccttgtgggcaaattggcgaaacggtcgatctcgatgaagccgtacaacgggcgctggaattcgctaaaaaggagggtaacacgctggtcatagtcaccgctgatcacgcccacgccagccagattgttgcgccggataccaaagctccgggcctcacccaggcgctaaataccaaagatggcgcagtgatggtgatgagttacgggaactccgaagaggattcacaagaacataccggcagtcagttgcgtattgcggcgtatggcccgcatgccgccaatgttgttggactgaccgaccagaccgatctcttctacaccatgaaagccgctctggggctgaaagggaggagacgatccaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcaccaagcttcagaagaactcgtcaagaaggcgatagaaggcgatgcgctgcgaatcgggagcggcgataccgtaaagcacgaggaagcggtcagcccattcgccgccaagctcctcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgttcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcgaagcatttatca

SEQ ID NO: 14 - DNA, 4389 bp, Artificial Sequence
  Description of Artificial Sequence Synthetic polynucleotide
gggttattgtctcatgagcggatacatatttgaatgtatttagaaaaataaacaaataggggttccgcgcacatttccccgaaaagtgccacgtctccaatgaacacgattaacatcgctaagaacgacttctctgacatcgaactggctgctatcccgttcaacactctggctgaccattacggtgagcgtttagctcgcgaacagttggcccttgagcatgagtcttacgagatgggtgaagcacgcttccgcaagatgtttgagcgtcaacttaaagctggtgaggttgcggataacgctgccgccaagcctctcatcactaccctactccctaagatgattgcacgcatcaacgactggtttgaggaagtgaaagctaagcgcggcaagcgcccgacagccttccagttcctgcaagaaatcaagccggaagccgtagcgtacatcaccattaagaccactctggcttgcctaaccagtgctgacaatacaaccgttcaggctgtagcaagcgcaatcggtcgggccattgaggacgaggctcgcttcggtcgtatccgtgaccttgaagctaagcacttcaagaaaaacgttgaggaacaactcaacaagcgcgtagggcacgtctacaagaaagcatttatgcaagttgtcgaggctgacatgctctctaagggtctactcggtggcgaggcgtggtcttcgtggcataaggaagactctattcatgtaggagtacgctgcatcgagatgctcattgagtcaaccggaatggttagcttacaccgccaaaatgctggcgtagtaggtcaagactctgagactatcgaactcgcacctgaatacgctgaggctatcgcaacccgtgcaggtgcgctggctggcatctctccgatgttccaaccttgcgtagttcctcctaagccgtggactggcattactggtggtggctattgggctaacggtcgtcgtcctctggcgctggtgcgtactcacagtaagaaagcactgatgcgctacgaagacgtttacatgcctgaggtgtacaaagcgattaacattgcgcaaaacaccgcatggaaaatcaacaagaaagtcctagcggtcgccaacgtaatcaccaagtggaagcattgtccggtcgaggacatccctgcgattgagcgtgaagaactcccgatgaaaccggaagacatcgacatgaatcctgaggctctcaccgcgtggaaacgtgctgccgctgctgtgtaccgcaaggacagggctcgcaagtctcgccgtatcagccttgagttcatgcttgagcaagccaataagtttgctaaccataaggccatctggttcccttacaacatggactggcgcggtcgtgtttacgccgtgtcaatgttcaacccgcaaggtaacgatatgaccaaaggactgcttacgctggcgaaaggtaaaccaatcggtaaggaaggttactactggctgaaaatccacggtgcaaactgtgcgggtgtcgataaggttccgttccctgagcgcatcaagttcattgaggaaaaccacgagaacatcatggcttgcgctaagtctccactggagaacacttggtgggctgagcaagattctccgttctgcttccttgcgttctgctttgagtacgctggggtacagcaccacggcctgagctataactgctcccttccgctggcgtttgacgggtcttgctctggcatccagcacttctccgcgatgctccgagatgaggtaggtggtcgcgcggttaacttgcttcctagtgagaccgttcaggacatctacgggattgttgctaagaaagtcaacgagattctacaagcagacgcaatcaatgggaccgataacgaagtagttaccgtgaccgatgagaacactggtgaaatctctgagaaagtcaagctgggcactaaggcactggctggtcaatggctggctcacggtgttactcgcagtgtgactaagcgttcagtcatgacgctggcttacgggtccaaagagttcggcttccgtcaacaagtgctggaagataccattcagccagctattgattccggcaagggtccgatgttcactcagccgaatcaggctgctggatacatggctaagctgatttgggaatctgtgagcgtgacggtggtagctgcggttgaagcaatgaactggcttaagtctgctgctaagctgctggctgctgaggtcaaagataagaagactggagagattcttcgcaagcgttgcgctgtgcattgggtaactcctgatggtttccctgtgtggcaggaatacaagaagcctattcagacgcgcttgaacctgatgttcctcggtcagttccgcttacagcctaccattaacaccaacaaagatagcgagattgatgcacacaaacaggagtctggtatcgctcctaactttgtacacagccaagacggtagccaccttcgtaagactgtagtgtgggcacacgagaagtacggaatcgaatcttttgcactgattcacgactccttcggtaccattccggctgacgctgcgaacctgttcaaagcagtgcgcgaaactatggttgacacatatgagtcttgtgatgtactggctgatttctacgaccagttcgctgaccagttgcacgagtctcaattggacaaaatgccagcacttccggctaaaggtaacttgaacctccgtgacatcttagagtcggacttcgcgttcgcggggaggagacgatccaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcaccaagcttcagaagaactcgtcaagaaggcgatagaaggcgatgcgctgcgaatcgggagcggcgataccgtaaagcacgaggaagcggtcagcccattcgccgccaagctcctcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgttcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcgaagcatttatca

SEQ ID NO: 15 - DNA, 4003 bp, Artificial Sequence
  Description of Artificial Sequence Synthetic polynucleotide
ccatcgaatggccagatgattaattcctaatttttgttgacactctatcattgatagagttattttaccactccctatcagtgatagagaaaagtgaaatgaatagttcgacaaaaattctagaaataattttgtttaactttaagaaggagatatacaaatgggagacgggatcccccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctccaccgcggtggcggccgctctagaactagtggatcccccgggctgcaggaattcgatatcaagcttatcgataccgtcgacctcgagggggggcccggtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacggatcccgtctcggggagcgcttggagccacccgcagttcgaaaaataataagcttgacctgtgaagtgaaaaatggcgcacattgtgcgacattttttttgtctgccgtttaccgctactgcgtcacggatctccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattgatagactggatggaggcggataaagttgcaggaccacttctgcgctcggcccttccggctggctggtttattgctgataaatctggagccggtgagcgtggctctcgcggtatcattgcagcactggggccagatggtaagccctcccgtatcgtagttatctacacgacggggagtcaggcaactatggatgaacgaaatagacagatcgctgagataggtgcctcactgattaagcattggtaggaattaatgatgtctcgtttagataaaagtaaagtgattaacagcgcattagagctgcttaatgaggtcggaatcgaaggtttaacaacccgtaaactcgcccagaagctaggtgtagagcagcctacattgtattggcatgtaaaaaataagcgggctttgctcgacgccttagccattgagatgttagataggcaccatactcacttttgccctttagaaggggaaagctggcaagattttttacgtaataacgctaaaagttttagatgtgctttactaagtcatcgcgatggagcaaaagtacatttaggtacacggcctacagaaaaacagtatgaaactctcgaaaatcaattagcctttttatgccaacaaggtttttcactagagaatgcattatatgcactcagcgcagtggggcattttactttaggttgcgtattggaagatcaagagcatcaagtcgctaaagaagaaagggaaacacctactactgatagtatgccgccattattacgacaagctatcgaattatttgatcaccaaggtgcagagccagccttcttattcggccttgaattgatcatatgcggattagaaaaacaacttaaatgtgaaagtgggtcttaaaagcagcataacctttttccgtgatggtaacttcactagtttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgacccgaca

SEQ ID NO: 16 - DNA, 3147 bp, Artificial Sequence
  Description of Artificial Sequence Synthetic polynucleotide
tcacggatctccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattgatagactggatggaggcggataaagttgcaggaccacttctgcgctcggcccttccggctggctggtttattgctgataaatctggagccggtgagcgtggctctcgcggtatcattgcagcactggggccagatggtaagccctcccgtatcgtagttatctacacgacggggagtcaggcaactatggatgaacgaaatagacagatcgctgagataggtgcctcactgattaagcattggtaggaattaatgatgtctcgtttagataaaagtaaagtgattaacagcgcattagagctgcttaatgaggtcggaatcgaaggtttaacaacccgtaaactcgcccagaagctaggtgtagagcagcctacattgtattggcatgtaaaaaataagcgggctttgctcgacgccttagccattgagatgttagataggcaccatactcacttttgccctttagaaggggaaagctggcaagattttttacgtaataacgctaaaagttttagatgtgctttactaagtcatcgcgatggagcaaaagtacatttaggtacacggcctacagaaaaacagtatgaaactctcgaaaatcaattagcctttttatgccaacaaggtttttcactagagaatgcattatatgcactcagcgcagtggggcattttactttaggttgcgtattggaagatcaagagcatcaagtcgctaaagaagaaagggaaacacctactactgatagtatgccgccattattacgacaagctatcgaattatttgatcaccaaggtgcagagccagccttcttattcggccttgaattgatcatatgcggattagaaaaacaacttaaatgtgaaagtgggtcttaaaagcagcataacctttttccgtgatggtaacttcactagtttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgacccgacaccatcgaatggccagatgattaattcctaatttttgttgacactctatcattgatagagttattttaccactccctatcagtgatagagaaaagtgaaatgaatagttcgacaaaaatctagaaataattttgtttaactttaagaaggagatatacagggagccacccgcaagcttgacctgtgaagtgaaaaatggcgcacattgtgcgacattttttttgtctgccgtttaccgctactgcg

SEQ ID NO: 17 - DNA, 3126 bp, Artificial Sequence
  Description of Artificial Sequence Synthetic polynucleotide
tcacggatctccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattgatagactggatggaggcggataaagttgcaggaccacttctgcgctcggcccttccggctggctggtttattgctgataaatctggagccggtgagcgtggctctcgcggtatcattgcagcactggggccagatggtaagccctcccgtatcgtagttatctacacgacggggagtcaggcaactatggatgaacgaaatagacagatcgctgagataggtgcctcactgattaagcattggtaggaattaatgatgtctcgtttagataaaagtaaagtgattaacagcgcattagagctgcttaatgaggtcggaatcgaaggtttaacaacccgtaaactcgcccagaagctaggtgtagagcagcctacattgtattggcatgtaaaaaataagcgggctttgctcgacgccttagccattgagatgttagataggcaccatactcacttttgccctttagaaggggaaagctggcaagattttttacgtaataacgctaaaagttttagatgtgctttactaagtcatcgcgatggagcaaaagtacatttaggtacacggcctacagaaaaacagtatgaaactctcgaaaatcaattagcctttttatgccaacaaggtttttcactagagaatgcattatatgcactcagcgcagtggggcattttactttaggttgcgtattggaagatcaagagcatcaagtcgctaaagaagaaagggaaacacctactactgatagtatgccgccattattacgacaagctatcgaattatttgatcaccaaggtgcagagccagccttcttattcggccttgaattgatcatatgcggattagaaaaacaacttaaatgtgaaagtgggtcttaaaagcagcataacctttttccgtgatggtaacttcactagtttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgacccgacaccatcgaatggccagatgattaattcctaatttttgttgacactctatcattgatagagttattttaccactccctatcagtgatagagaaaagtgaaatgaatagttcgacaaaaatctagataacgagggcaaaagggagccacccgcaagcttgacctgtgaagtgaaaaatggcgcacattgtgcgacattttttttgtctgccgtttaccgctactgcg

SEQ ID NO: 18 - DNA, 2766 bp, Artificial Sequence
  Description of Artificial Sequence Synthetic polynucleotide
ataaccccttggggcctctaaacgggtcttgaggggttttttgctgaaaggaggaactatatccggatctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggttctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggatgagcgcccaatacgcaaaccgcctctccccgcgcgttggccgattcattaatgcaggatctcgatcccgcgaaattaatacgactcactatagggaggccacaacggtttccctctagaaataattttgtttaactttaagaaggagatatacagggagccacccgcaagcttgatccggctgctaacaaagcccgaaaggaagctgagttggctgctgccaccgctgagcaataactagc

SEQ ID NO: 19 - DNA, 5358 bp, Artificial Sequence
  Description of Artificial Sequence Synthetic polynucleotide
gttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaggagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagaacttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctattccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggtctagacccacgggagccacccgcaagcttgcggccgcagatctagcttaagtttaaaccgctgatcagcctcgactgtgccttcta

SEQ ID NO: 20 - DNA, 7108 bp, Artificial Sequence
  Description of Artificial Sequence Synthetic polynucleotide
aaaataatgaacaatgccaaaaatcatgtagctgcccaacggggtgtaacagcgacgacaaatgcccctgcggtaacaagtctgaagaaaccaagaagtcatgctgctctgggaaatgaaacgaatagtctttaatatattcatctaactatttgctgtttttaatttttaaaaggagaaggaagtttaatcgacgattctactcagtttgagtacacttatgtattttgtttagatactttgttaatttataggtatacgttaataattaagaaaaggaaataaagtatctccatatgtcgccccaagaataaaatattattaccaaattctagtttgcctaacttacaactctgtatagaatccccagatttcgaataaaaaaaaaaaaagctattcatggtaccgcgatgtagtaaaactagctagaccgagaaagagactagaaatgcaaaaggcacttctacaatggctgccatcattattatccgatgtgacgctgcatttttttttttttttttttttttttttttttttttttgtgtacaaatatcataaaaaaagagaatctttttaagcaaggattttcttaacttcttcggcgacagcatcaccgacttcggtggtactgttggaaccacctaaatcaccagttctgatacctgcatccaaaacctttttaactgcatcttcaatggctttaccttcttcaggcaagttcaatgacaatttcaacatcattgcagcagacaagatagtggcgatagggttgaccttattctttggcaaatctggagcggaaccatggcatggttcgtacaaaccaaatgcggtgttcttgtctggcaaagaggccaaggacgcagatggcaacaaacccaaggagcctgggataacggaggcttcatcggagatgatatcaccaaacatgttgctggtgattataataccatttaggtgggttgggttcttaactaggatcatggcggcagaatcaatcaattgatgttgaaccttcaatgtaggaaattcgttcttgatggtttcctccacagtttttctccataatcttgaagaggccaaaacattagctttatccaaggaccaaataggcaatggtggctcatgttgtagggccatgaaagcggccattcttgtgattctttgcacttctggaacggtgtattgttcactatcccaagcgacaccatcaccatcgtcttcctttctcttaccaaagtaaatacctcccactaattctctgacaacaacgaagtcagtacctttagcaaattgtggcttgattggagataagtctaaaagagagtcggatgcaaagttacatggtcttaagttggcgtacaattgaagttctttacggatttttagtaaaccttgttcaggtctaacactacctgtaccccatttaggaccacccacagcacctaacaaaacggcatcagccttcttggaggcttccagcgcctcatctggaagtggaacacctgtagcatcgatagcagcaccaccaattaaatgattttcgaaatcgaacttgacattggaacgaacatcagaaatagctttaagaaccttaatggcttcggctgtgatttcttgaccaacgtggtcacctggcaaaacgacgatcttcttaggggcagacattacaatggtatatccttgaaatatatataaaaaaaaaaaaaaaaaaaaatgcagcttctcaatgatattcgaatacgctttgaggagatacagcctaatatccgacaaactgttttacagatttacgatcgtacttgttacccatcattgaattttgaacatccgaacctgggagttttccctgaaacagatagtatatttgaacctgtataataatatatagtctagcgctttacggaagacaatgtatgtatttcggttcctggagaaactattgcatctattgcataggtaatcttgcacgtcgcatccccggttcattttctgcgtttccatcttgcacttcaatagcatatctttgttaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatataggggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcgatccgatgataagctgtcaaacatgagaattgggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtcgcccttgtcatctaaacccacaccgggtgtcataatcaaccaatcgtaaccttcatctcttccacccatgtctctttgagcaataaagccgataacaaaatctttgtcgctcttggcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcatgtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaggaaaccgaaatcaaaaaaaagaataaaaaaaaaatgatgaattgaaaagctaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcgaaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgcatgtcttttgctggcatttctcctagaagcaaaaagagcgatgcgtcttttccgctgaaccgttccagcaaaaaagactaccaacgcaatatggattgtcagaatcatataaaagagaagcaaataactccttgtcttgtatcaattgcattataatatcttcttgttagtgcaatatcatatagaagtcatcgaaatagatattaagaaaaacaaactgtacaatcaatcatcacatcaatcatcacataaaatattcagcgaattgaatctagacccacgcttaattcattaacttccaaaatgaaggtcatgagtgccaatgccaatgtggtagctgca

SEQ ID NO: 21 - DNA, 690 bp, Artificial Sequence
  Description of Artificial Sequence Synthetic polynucleotide
agagacgctgcagcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctggagctccaccgcggtggcggccgctctagaactagtggatcccccgggctgcaggaattcgatatcaagcttatcgataccgtcgacctcgagggggggcccggtacccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacctcgagcgtctca

SEQ ID NO: 22 - DNA, 2475 bp, Artificial Sequence
  Description of Artificial Sequence Synthetic polynucleotide
gggttattgtctcatgagcggatacatatttgaatgtatttagaaaaataaacaaataggggttccgcgcacatttccccgaaaagtgctggacccatctagaaaggaacgtctccaatgagaagagcctgcagcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctggagctccaccgcggtggcggccgctctagaactagtggatcccccgggctgcaggaattcgatatcaagcttatcgataccgtcgacctcgagggggggcccggtacccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacctcgaggctcttctgggaggagacgaaggaaaagcttgtcgagggcaatccaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgattccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcaccgagcttcagaagaactcgtcaagaaggcgatagaaggcgatgcgctgcgaatcgggagcggcgataccgtaaagcacgaggaagcggtcagcccattcgccgccaagctcctcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgttcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcgaagcatttatca

SEQ ID NO: 23 - DNA, 2471 bp, Artificial Sequence
  Description of Artificial Sequence Synthetic polynucleotide
gggttattgtctcatgagcggatacatatttgaatgtatttagaaaaataaacaaataggggttccgcgcacatttccccgaaaagtgctggacccatctagaaaggaacgtctccaatgagactcctgcagcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctggagctccaccgcggtggcggccgctctagaactagtggatcccccgggctgcaggaattcgatatcaagcttatcgataccgtcgacctcgagggggggcccggtacccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacctcgaggagtcagggaggagacgaaggaaaagcttgtcgagggcaatccaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgattccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcaccgagcttcagaagaactcgtcaagaaggcgatagaaggcgatgcgctgcgaatcgggagcggcgataccgtaaagcacgaggaagcggtcagcccattcgccgccaagctcctcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgttcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcgaagcatttatca

SEQ ID NO: 24 - DNA, 2548 bp, Artificial Sequence
  Description of Artificial Sequence Synthetic polynucleotide
gggttattgtctcatgagcggatacatatttgaatgtatttagaaaaataaacaaataggggttccgcgcacatttccccgaaaagtgccagctcttcaatgagagacgctgcagcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctggagctccaccgcggtggcggccgctctagaactagtggatcccccgggctgcaggaattcgatatcaagcttatcgataccgtcgacctcgagggggggcccggtacccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacctcgagcgtctcagggagctaacgagggcaaaaaatggaagagctccaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcaccaagcttgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatca

SEQ ID NO: 25 - DNA, 2523 bp, Artificial Sequence
  Description of Artificial Sequence Synthetic polynucleotide
gggttattgtctcatgagcggatacatatttgaatgtatttagaaaaataaacaaataggggttccgcgcacatttccccgaaaagtgccagctcttcaaatgagagacgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctggagctccaccgcggtggcggccgctctagaactagtggatcccccgggctgcaggaattcgatatcaagcttatcgataccgtcgacctcgagggggggcccggtacccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgctcctttcgctttcttcccttcctttctcgccacgttcgccggctttccccgtcaagctctaaatcaggggctccctttagggttccgatttagtgctttacggcacctcgaccccaaaaaacttgattagggtgatggttcacctcgagcgtctcagggagaagagctccaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcaccaagcttgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatca

SEQ ID NO: 26 - DNA, 38 bp, Artificial Sequence
  Description of Artificial Sequence Synthetic oligonucleotide
cgaagagccgctcgaaataatattcgagcggctcttcg

SEQ ID NO: 27 - DNA, 11 bp, Artificial Sequence
  Description of Artificial Sequence Synthetic oligonucleotide
nnnngaagagc

SEQ ID NO: 28 - DNA, 18 bp, Artificial Sequence
  Description of Artificial Sequence Synthetic oligonucleotide
ngctcttcgcgaagagcn

SEQ ID NO: 29 - DNA, 44 bp, Artificial Sequence
  Description of Artificial Sequence Synthetic oligonucleotide
ngctcttcnnnnnngactcnnnnnngagtcnnnnnngaagagcn

SEQ ID NO: 30 - DNA, 11 bp, Artificial Sequence
  Description of Artificial Sequence Synthetic oligonucleotide
nnnnngagacg

SEQ ID NO: 31 - DNA, 12 bp, Artificial Sequence
  Description of Artificial Sequence Synthetic oligonucleotide
cacnnnnnntcc

SEQ ID NO: 32 - DNA, 16 bp, Artificial Sequence
  Description of Artificial Sequence Synthetic oligonucleotide
gctaacgagggcaaaa

SEQ ID NO: 33 - DNA, 44 bp, Artificial Sequence
  Description of Artificial Sequence Synthetic oligonucleotide
catcgaagagccgctcgaaataatattcgagcggctcttcgatg

SEQ ID NO: 34 - DNA, 44 bp, Artificial Sequence
  Description of Artificial Sequence Synthetic oligonucleotide
gggcgaagagccgctcgaaataatattcgagcggctcttcgccc

SEQ ID NO: 35 - DNA, 41 bp, Artificial Sequence
  Description of Artificial Sequence Synthetic oligonucleotide
catcgaagagccgctcgaaataatattcgagcggctcttcg

SEQ ID NO: 36 - DNA, 41 bp, Artificial Sequence
  Description of Artificial Sequence Synthetic oligonucleotide
gggcgaagagccgctcgaaataatattcgagcggctcttcg

SEQ ID NO: 37 - DNA, 41 bp, Artificial Sequence
  Description of Artificial Sequence Synthetic oligonucleotide
ncgtctcnaatgngaagagcngctcttcngggangagacgn

SEQ ID NO: 38 - DNA, 12 bp, Artificial Sequence
  Description of Artificial Sequence Synthetic oligonucleotide
cattngagacgn

SEQ ID NO: 39 - DNA, 12 bp, Artificial Sequence
  Description of Artificial Sequence Synthetic oligonucleotide
gggangagacgn

SEQ ID NO: 40 - DNA, 12 bp, Artificial Sequence
  Description of Artificial Sequence Synthetic oligonucleotide
ncgtctcnaatg

SEQ ID NO: 41 - DNA, 23 bp, Artificial Sequence
  Description of Artificial Sequence Synthetic oligonucleotide
aatgngagacgncgtctcnggga

SEQ ID NO: 42 - DNA, 12 bp, Artificial Sequence
  Description of Artificial Sequence Synthetic oligonucleotide
ttttngagacgn

SEQ ID NO: 43 - DNA, 12 bp, Artificial Sequence
  Description of Artificial Sequence Synthetic oligonucleotide
ncgtctcntttt

SEQ ID NO: 44 - DNA, 12 bp, Artificial Sequence
  Description of Artificial Sequence Synthetic oligonucleotide
gggangagacgn

SEQ ID NO: 45 - DNA, 11 bp, Artificial Sequence
  Description of Artificial Sequence Synthetic oligonucleotide
cgtctcaaatg

SEQ ID NO: 46 - DNA, 30 bp, Artificial Sequence
  Description of Artificial Sequence Synthetic oligonucleotide
gggaggagaccnggtctcagggaggagacg

SEQ ID NO: 47 - DNA, 30 bp, Artificial Sequence
  Description of Artificial Sequence Synthetic oligonucleotide
gctcttcaatgtgagacgncgtctcaggga

SEQ ID NO: 48 - DNA, 11 bp, Artificial Sequence
  Description of Artificial Sequence Synthetic oligonucleotide
taaggaagagc

SEQ ID NO: 49 - DNA, 11 bp, Artificial Sequence
  Description of Artificial Sequence Synthetic oligonucleotide
gctcttcaatg

SEQ ID NO: 50 - DNA, 23 bp, Artificial Sequence
  Description of Artificial Sequence Synthetic oligonucleotide
aatgtgagaccnggtctcaggga

SEQ ID NO: 51 - DNA, 11 bp, Artificial Sequence
  Description of Artificial Sequence Synthetic oligonucleotide
taaggaagagc

SEQ ID NO: 52 - DNA, 18 bp, Artificial Sequence
  Description of Artificial Sequence Synthetic oligonucleotide
ggtctcaaatgggagacg

SEQ ID NO: 53 - DNA, 18 bp, Artificial Sequence
  Description of Artificial Sequence Synthetic oligonucleotide
cgtctcagggaggagacc

SEQ ID NO: 54 - DNA, 11 bp, Artificial Sequence
  Description of Artificial Sequence Synthetic oligonucleotide
gctcttcaatg

SEQ ID NO: 55 - DNA, 11 bp, Artificial Sequence
  Description of Artificial Sequence Synthetic oligonucleotide
aatgtgagacg

SEQ ID NO: 56 - DNA, 11 bp, Artificial Sequence
  Description of Artificial Sequence Synthetic oligonucleotide
cgtctcaggga

SEQ ID NO: 57 - DNA, 11 bp, Artificial Sequence
  Description of Artificial Sequence Synthetic oligonucleotide
taaggaagagc

SEQ ID NO: 58 - DNA, 44 bp, Artificial Sequence
  Description of Artificial Sequence Synthetic oligonucleotide
aagcgaagagccgctcgaaataatattcgagcggctcttcgctt

SEQ ID NO: 59 - DNA, 44 bp, Artificial Sequence
  Description of Artificial Sequence Synthetic oligonucleotide
cttcgaagagccgctcgaaataatattcgagcggctcttcgaag

SEQ ID NO: 60 - DNA, 41 bp, Artificial Sequence
  Description of Artificial Sequence Synthetic oligonucleotide
cttcgaagagccgctcgaaataatattcgagcggctcttcg

SEQ ID NO: 61 - DNA, 41 bp, Artificial Sequence
  Description of Artificial Sequence Synthetic oligonucleotide
aagcgaagagccgctcgaaataatattcgagcggctcttcg

SEQ ID NO: 62 - DNA, 23 bp, Artificial Sequence
  Description of Artificial Sequence Synthetic oligonucleotide
aatgngagaccnggtctcnggga

SEQ ID NO: 63 - DNA, 25 bp, Artificial Sequence
  Description of Artificial Sequence Synthetic oligonucleotide
ncacnnnnnntccnnnnnnnaaatg

SEQ ID NO: 64 - DNA, 26 bp, Artificial Sequence
  Description of Artificial Sequence Synthetic oligonucleotide
ggggannnnnnnncacnnnnnntccn

SEQ ID NO: 65 - DNA, 32 bp, Artificial Sequence
  Description of Artificial Sequence Synthetic oligonucleotide
nnnnnnnncacnnnnnntccnnnnnnnnnnnn

SEQ ID NO: 66 - DNA, 32 bp, Artificial Sequence
  Description of Artificial Sequence Synthetic oligonucleotide
nnnnnnnggannnnnngtgnnnnnnnntcccc

SEQ ID NO: 67 - DNA, 32 bp, Artificial Sequence
  Description of Artificial Sequence Synthetic oligonucleotide
nnnnnnnncacnnnnnntccnnnnnnnaaatg

SEQ ID NO: 68 - DNA, 32 bp, Artificial Sequence
  Description of Artificial Sequence Synthetic oligonucleotide
nnnnnnnggannnnnngtgnnnnnnnnnnnnn

SEQ ID NO: 69 - DNA, 37 bp, Artificial Sequence
  Description of Artificial Sequence Synthetic oligonucleotide
aaatgnnnnnnnncacnnnnnntccnnnnnnngggga

SEQ ID NO: 70 - DNA, 37 bp, Artificial Sequence
  Description of Artificial Sequence Synthetic oligonucleotide
ncgtctcnnnnnngactcngagtcnnnnnngagacgn

SEQ ID NO: 71 - DNA, 12 bp, Artificial Sequence
  Description of Artificial Sequence Synthetic oligonucleotide
ncgtctcntccc

SEQ ID NO: 72 - DNA, 12 bp, Artificial Sequence
  Description of Artificial Sequence Synthetic oligonucleotide
cattngagacgn

SEQ ID NO: 73 - DNA, 44 bp, Artificial Sequence
  Description of Artificial Sequence Synthetic oligonucleotide
ngctcttcaatgngagacgncgtctcnggganaatngaagagcn

SEQ ID NO: 74 - DNA, 12 bp, Artificial Sequence
  Description of Artificial Sequence Synthetic oligonucleotide
ngctcttcaatg

SEQ ID NO: 75 - DNA, 17 bp, Artificial Sequence
  Description of Artificial Sequence Synthetic oligonucleotide
ggganaatngaagagcn

SEQ ID NO: 76 - DNA, 40 bp, Artificial Sequence
  Description of Artificial Sequence Synthetic oligonucleotide
ngctcttcnaatgngagacgncgtctcngggagaagagcn

SEQ ID NO: 77 - DNA, 13 bp, Artificial Sequence
  Description of Artificial Sequence Synthetic oligonucleotide
ngctcttcnaatg

SEQ ID NO: 78 - DNA, 12 bp, Artificial Sequence
  Description of Artificial Sequence Synthetic oligonucleotide
gggagaagagcn

SEQ ID NO: 79 - DNA, 46 bp, Artificial Sequence
  Description of Artificial Sequence Synthetic oligonucleotide
gggagcggtggcggtagcggtggcggttccggtggcggtagcaatg

SEQ ID NO: 80 - protein, 14 aa, Artificial Sequence
  Description of Artificial Sequence Synthetic peptide
GSGGGSGGGSGGGS

SEQ ID NO: 81 - DNA, 36 bp, Artificial Sequence
  Description of Artificial Sequence Synthetic oligonucleotide
gggagcgcttggagccacccgcagttcgaaaaataa

SEQ ID NO: 82 - protein, 11 aa, Artificial Sequence
  Description of Artificial Sequence Synthetic peptide
GSAWSHPQFEK

SEQ ID NO: 83 - DNA, 46 bp, Artificial Sequence
  Description of Artificial Sequence Synthetic oligonucleotide
aatggctagcgcatggagtcatcctcaattcgaaaaatccggaatg

SEQ ID NO: 84 - protein, 15 aa, Artificial Sequence
  Description of Artificial Sequence Synthetic peptide
MASAWSHPQFEKSGM

SEQ ID NO: 85 - DNA, 13 bp, Artificial Sequence
  Description of Artificial Sequence Synthetic oligonucleotide
aatgtcccctata

SEQ ID NO: 86 - protein, 4 aa, Artificial Sequence
  Description of Artificial Sequence Synthetic peptide
MSPI

SEQ ID NO: 87 - DNA, 60 bp, Artificial Sequence
  Description of Artificial Sequence Synthetic oligonucleotide
cctccaaaaatgtccggaggtggcggtgggagcctggaagttctgttccaggggccaatg

SEQ ID NO: 88 - protein, 20 aa, Artificial Sequence
  Description of Artificial Sequence Synthetic peptide
PPKMSGGGGGSLEVLFQGPM

SEQ ID NO: 89 - DNA, 30 bp, Artificial Sequence
  Description of Artificial Sequence Synthetic oligonucleotide
gggagcgctcaccatcaccatcaccattaa

SEQ ID NO: 90 - protein, 9 aa, Artificial Sequence
  Description of Artificial Sequence Synthetic peptide
GSAHHHHHH

SEQ ID NO: 91 - DNA, 37 bp, Artificial Sequence
  Description of Artificial Sequence Synthetic oligonucleotide
aatggctagccatcaccatcaccatcactccggaatg

SEQ ID NO: 92 - protein, 12 aa, Artificial Sequence
  Description of Artificial Sequence Synthetic peptide
MASHHHHHHSGM

SEQ ID NO: 93 - DNA, 96 bp, Artificial Sequence
  Description of Artificial Sequence Synthetic oligonucleotide
gggagcgcttggagccacccgcagttcgaaaaaggtggaggttctggcggtggatcgggaggttcagcgtggagccacccgcagttcgagaaataa

SEQ ID NO: 94 - protein, 31 aa, Artificial Sequence
  Description of Artificial Sequence Synthetic polypeptide
GSAWSHPQFEKGGGSGGGSGGSAWSHPQFEK

SEQ ID NO: 95 - DNA, 106 bp, Artificial Sequence
  Description of Artificial Sequence Synthetic polynucleotide
aatggctagcgcatggagtcatcctcaattcgagaaaggtggaggttctggcggtggatcgggaggttcagcgtggagccacccgcagttcgaaaaatccggaatg

SEQ ID NO: 96 - protein, 35 aa, Artificial Sequence
  Description of Artificial Sequence Synthetic polypeptide
MASAWSHPQFEKGGGSGGGSGGSAWSHPQFEKSGM

SEQ ID NO: 97 - DNA, 13 bp, Artificial Sequence
  Description of Artificial Sequence Synthetic oligonucleotide
aatgaaaaagaca

SEQ ID NO: 98 - protein, 4 aa, Artificial Sequence
  Description of Artificial Sequence Synthetic peptide
MKKT

SEQ ID NO: 99 - DNA, 15 bp, Artificial Sequence
  Description of Artificial Sequence Synthetic oligonucleotide
gcgcaggccgcaatg

SEQ ID NO: 100 - protein, 5 aa, Artificial Sequence
  Description of Artificial Sequence Synthetic peptide
AQAAM

SEQ ID NO: 101 - DNA, 57 bp, Artificial Sequence
  Description of Artificial Sequence Synthetic oligonucleotide
gcgcaggccgcaatggctagcgcatggagtcatcctcaattcgaaaaatccggaatg

SEQ ID NO: 102 - protein, 19 aa, Artificial Sequence
  Description of Artificial Sequence Synthetic peptide
AQAAMASAWSHPQFEKSGM

SEQ ID NO: 103 - DNA, 117 bp, Artificial Sequence
  Description of Artificial Sequence Synthetic polynucleotide
gcgcaggccgcaatggctagcgcatggagtcatcctcaattcgagaaaggtggaggttctggcggtggatcgggaggttcagcgtggagccacccgcagttcgaaaaatccggaatg

SEQ ID NO: 104 - protein, 39 aa, Artificial Sequence
  Description of Artificial Sequence Synthetic polypeptide
AQAAMASAWSHPQFEKGGGSGGGSGGSAWSHPQFEKSGM

SEQ ID NO: 105 - DNA, 13 bp, Artificial Sequence
  Description of Artificial Sequence Synthetic oligonucleotide
aatgagggcctgg

SEQ ID NO: 106 - protein, 4 aa, Artificial Sequence
  Description of Artificial Sequence Synthetic peptide
MRAW

SEQ ID NO: 107 - DNA, 15 bp, Artificial Sequence
  Description of Artificial Sequence Synthetic oligonucleotide
gctctggcagcaatg

SEQ ID NO: 108 - protein, 5 aa, Artificial Sequence
  Description of Artificial Sequence Synthetic peptide
ALAAM

SEQ ID NO: 109 - DNA, 117 bp, Artificial Sequence
  Description of Artificial Sequence Synthetic polynucleotide
gctctggcagcaatggctagcgcatggagtcatcctcaattcgagaaaggtggaggttctggcggtggatcgggaggttcagcgtggagccacccgcagttcgaaaaatccggaatg

SEQ ID NO: 110 - protein, 39 aa, Artificial Sequence
  Description of Artificial Sequence Synthetic polypeptide
ALAAMASAWSHPQFEKGGGSGGGSGGSAWSHPQFEKSGM

SEQ ID NO: 111 - DNA, 48 bp, Artificial Sequence
  Description of Artificial Sequence Synthetic oligonucleotide
gctctggcagcaatggctagccatcaccatcaccatcactccggaatg

SEQ ID NO: 112 - protein, 16 aa, Artificial Sequence
  Description of Artificial Sequence Synthetic peptide
ALAAMASHHHHHHSGM

SEQ ID NO: 113 - protein, 6 aa, Artificial Sequence
  Description of Artificial Sequence Synthetic 6xHis tag
HHHHHH

(19) United States
(12) Patent Application Publication
(10) Pub. No.: US 2010/0291633 A1
(43) Pub. Date: Nov. 18, 2010

(54) METHOD OF CLONING AT LEAST ONE NUCLEIC ACID MOLECULE OF INTEREST USING TYPE IIS RESTRICTION ENDONUCLEASES, AND CORRESPONDING CLONING VECTORS, KITS AND SYSTEM USING TYPE IIS RESTRICTION ENDONUCLEASES

(76) Inventors: Thorsten Selmer, Bonn-Buschdorf (DE); Olaf Pinkenburg, Marburg (DE)

Correspondence Address: BioTechnology Law Group;12707 High Bluff Drive, Suite 200, San Diego, CA, 92130-2037, US

(21) Appl. No.: 12/525,905

(22) PCT Filed: Feb. 5, 2008
(86) PCT No.: PCT/EP08/51396

§ 371 Date: May 11, 2010

(30) Foreign Application Priority Data
Sep. 3, 2007 (EP) 07017230.9

(51) Int. Cl. C12P 19/34; C12N 15/63

(52) U.S. Cl. 435/91.1; 435/320.1

(57) ABSTRACT

The present invention refers to methods of (sub)cloning at least one nucleic acid molecule of interest. One embodiment relates to a method of (sub)cloning at least one nucleic acid molecule of interest comprising a) providing at least one (replicable) Entry vector into which the at least one nucleic acid molecule of interest is to be inserted, wherein the at least one Entry vector carries two recognition sites for at least one first type IIS and/or type IIS like restriction endonuclease and wherein said at least one nucleic acid molecule of interest can be excised from the at least one Entry vector at two combinatorial sites with one (same) or more (different) cohesive ends that are formed by the at least one first type IIS or type IIS like restriction endonuclease, and b) providing an Acceptor vector, into which the at least one nucleic acid molecule of interest is transferred from the at least one Entry vector carrying the at least one nucleic acid molecule of interest, wherein said Acceptor vector comprises at least one recognition site for at least one second type IIS restriction endonuclease and/or at least one recognition sites for at least one type IIS like restriction endonuclease, and wherein said Acceptor vector provides two combinatorial sites identical to the two combinatorial sites present in the Entry vector. The inventions also relates respective cloning vector and kits.

### CROSS-REFERENCE TO RELATED APPLICATIONS

The present application claims the benefit of priority of U.S. provisional application No. 60/888,216 filed Feb. 5, 2007, U.S. provisional application No. 60/889,429 filed Feb. 12, 2007, U.S. provisional application No. 60/950,559 filed Jul. 18, 2007, European patent application 07017230 filed Sep. 3, 2007 and U.S. provisional application No. 60/969,781 filed Sep. 4, 2007, the contents of each being hereby incorporated by reference in its entirety for all purposes.

### FIELD OF THE INVENTION

The invention is generally in the field of polynucleotide manipulation techniques, particularly amplification and cloning techniques. The invention provides, for example, a new generic cloning method, respective cloning vectors and a cloning kit allowing the precise and directed recombination of nucleic acid molecules, e.g., from a Donor vector into one Acceptor vector or in parallel into a multitude of Acceptor vectors thereby bringing the nucleic acid molecule into different genetic surroundings which are pre-defined by each Acceptor vector. The invention also provides a new and elegant way of mutating a nucleic acid molecule of interest. In another aspect of the invention, directed assembly of a multitude of nucleic acid molecules is enabled in a one tube reaction or sequentially by generating intermediate Entry vectors thereby providing new efficient means for generic plasmid construction. Such an efficient means for generic plasmid construction by combining individual nucleic acid molecules is for example useful for the fast development of vectors to be applied in diagnosis and therapy of human or animal diseases. Examples are gene therapy vectors, e.g. to substitute inherited absence of important protein factors, and DNA vaccination vectors, e.g. to express antigens in vivo for immunization against pathogens and other targets.

### BACKGROUND OF THE INVENTION

Genomics and proteomics are rapidly evolving fields since the genomes of many organisms have been sequenced and mapped. One of the challenges in the post-genomic era is functional annotation of genes and gene products, i.e. proteins, and their dynamic interaction for the generation of cellular functions.

Gene and gene product analysis often involves the initial cloning of the target nucleic acid molecule via PCR into a first cloning vector for sequence confirmation. Then, subcloning into a genetic environment which enables the desired manipulations or studies often becomes necessary. For example, but without limited thereto, subcloning is necessary when genetic studies are to be performed in different host organisms, if gene expression is to be tested in different host organisms or under the control of different promoters, or if different labels (tags) for affinity purification or for fluorescent labelling have to be tested.

When e.g. the desired manipulation is to express the gene in order to generate/produce the gene product then the gene has to be placed under the control of a suitable promoter in a vector that functions in a suitable expression host. Examples for commonly used expression hosts are bacteria, yeasts, insect and mammalian cells. For each host several promoters are known with different functionalities lying primarily in different strength or in different means for regulation. Examples for promoters commonly used in e.g. bacteria are the arabinose, T7, tetracycline, lac and T5 promoter and the like. If the gene product is further intended to be purified, the fusion of particular affinity tag(s) for the application of facilitated purification scheme(s) may be advantageous. Examples for common affinity tags are the oligohistidine-tags, for example, hexahistidine tags, the FLAG-tag, the glutathione-S-transferase tag (GST-tag) and the different versions of strepavidin binding tags, for example those marketed under the trademark STREP-TAG®, and the like. It is often desirable to compare amino terminal and carboxy terminal affinity tag fusions regarding activity, solubility, stability, and the like.

Thus, many tools for the expression and purification of a recombinant protein are currently available. Due to the heterogenic nature of proteins, however, it is impossible to predict which combination of these tools will perform best in a defined situation, and often many have to be tried in order to identify an optimal solution for a given problem. This example makes clear that there is a significant need for screening which is extremely facilitated when having efficient subcloning systems to recombine nucleic acid molecules at hand.

Traditional subcloning strategies are slow and inefficient. A way to improve traditional subcloning is attempted by the GATEWAY™; system marketed by Invitrogen. This system uses site directed recombination as described in U.S. Pat. No. 5,888,732. Briefly, the desired gene is initially cloned in an entry vector where it may be verified by sequencing when PCR has been used during cloning. Then, an enzymatic in vitro recombination reaction is used to transfer the gene into different destination vectors in order to bring the gene into different genetic surroundings in parallel by one step only. This strategy uses distinct phage lambda derived recombination sites at the 5′ and the 3′ end of the gene fragment (attL), which are provided by the entry vector. During transfer reaction, these sites are directionally recombined with compatible recombination sites of destination vectors (attR) operatively linked to functional genetic elements like, e.g., host specific promoters or affinity tags and attB sites will remain in the final product separating the gene from the functional elements. A similar system called CREATOR™ using cre/lox recombination sites from phage P1 has been developed and marketed by Clontech.

This strategy using recombination sites at the 5′ and the 3′ end of the gene fragment/nucleic acid molecule of interest avoids multiple subcloning steps which typically consist of (i) digestion the DNA of interest with one or two restriction enzymes; (ii) gel purification of the DNA segment of interest when known; (iii) preparation of the vector by cutting with appropriate restriction enzymes, treating with alkaline phosphatase, gel purification etc., as appropriate; (iv) ligation the DNA segment to vector, with appropriate controls to estimate background of uncut and self-ligated vector; (v) introduction of the resulting vector into an E. coli host cell; (vi) picking selected colonies and growing small cultures overnight; (vii) making DNA minipreparation; and (viii) analysis of the isolated plasmid on agarose gels (often after diagnostic restriction enzyme digestions) or by PCR.

Although subcloning efficiency towards traditional strategies is improved by the GATEWAY™ and CREATOR™ cloning systems, limitations remain. They primarily lie in the availability and length of recombination sites, especially when more than 2 fragments have to be assembled. These limitations are difficult to overcome, since only a very limited number of pre-defined recombination sites are known. Moreover, these pre-defined recombination sites require extensive changes within a given or desired target nucleic acid molecule at the point of fusion, since these recombination sites have a significant sequence length (the loxP site is commonly 34 bases and attB is 25 bases long). One alternative cloning system is described in the German Offenlegungsschrift DE 103 37 407. Therein an entry vector comprising two recognition sites for a type IIS restriction endonuclease and an acceptor vector comprising recognition sites for a regular type IIP restriction enzyme are used for subcloning a nucleic acid of interest.

Directionality is an important factor for efficiency. Therefore, the use of non compatible recombination sites at the 3′ and 5′ ends of the nucleic acid molecule to be investigated is essential. Whenever multiple recombination sites are considered, a directed assembly of various individual nucleic acid molecules is only possible if (i) the recombination site at either end of a molecule matches the needs for recombination with the adjacent partner and (ii) if the number of different recombination sites is at least equal or larger than the number of fragments to be combined. This problem becomes even more complex whenever multiple nucleic acid molecules have to be combined simultaneously (e.g. when the time consuming successive assembly is to be avoided) and must recombine in ordered (e.g. the natural order of promotor, RBS and start codon) and directed way (e.g. the in frame fusion of gene with a N- or C-terminal tag). The number of problems increases exponentially when for example several genes encoding subunits of e.g. an enzyme complex are intended to be embedded in a polycistronic operon or, ultimatively, when whole vectors are intended to be assembled by the use of functional nucleic acid molecules pre-cloned in donor vectors.

Another important problem is the retention of all of the recombination sites in the newly assembled vector in the above described recombination systems, as they cause an alteration or function which may be not desired. Such an alteration or function may for example be, but not limited thereto, encoding defined amino acids that modify a target gene product thereby potentially altering its function and impairing functional analysis or introducing a slippery codon inducing frameshifts during translation (see for example Belfield et al., Nucleic Acid Research 35, pages 1322-1332, 2007, The gateway pDEST17 expression vector encodes a −1 ribosomal frameshifting sequence). The method described by Rebatchouk et al., Proc. Natl. Acad. Sci. USA, Vol 93, pages 10891-10896, 1996 and termed nucleic acid ordered molecule assembly with directionality (NOMAD) tries to overcome this problem.

However, in view of the foregoing limitations of current recombinant DNA technology, there is still a need for a method for conveniently manipulating nucleic acid molecules without having to rely on natural occurring recombination sites. Such a method should allow efficient subcloning and recombination of nucleic acid molecules without the need for substantial modification. Additionally, such a method should allow the directed assembly of a multitude of nucleic acid molecules.

The present invention meets these needs by the feature(s) as defined in the respective independent claims.

### SUMMARY OF THE INVENTION

Thus, in a first aspect the invention provides a method of (sub)cloning at least one nucleic acid molecule of interest comprising
a) providing at least one (replicable) Entry vector into which the at least one nucleic acid molecule of interest is to be inserted, wherein the at least one Entry vector carries two recognition sites for at least one first type IIS restriction endonuclease and wherein said at least one nucleic acid molecule of interest can be excised from the at least one Entry vector at two combinatorial sites with one (same) or more (different) cohesive ends that are formed by the at least one first type IIS restriction endonuclease, and
b) providing an Acceptor vector, into which the at least one nucleic acid molecule of interest is transferred from the at least one Entry vector carrying the at least one nucleic acid molecule of interest, wherein said Acceptor vector comprises at least one recognition site for at least one second type IIS restriction endonuclease, and wherein said Acceptor vector provides two combinatorial sites identical to the two combinatorial sites present in the Entry vector.

In other words, the first aspect of the invention provides a method of (sub)cloning at least one nucleic acid molecule of interest comprising
a) providing at least one (replicable) Entry vector into which the at least one nucleic acid molecule of interest is to be inserted, wherein the at least one Entry vector carries two combinatorial sites with associated recognition sites for at least one first type IIS restriction endonuclease and wherein said at least one nucleic acid molecule of interest can be excised from the at least one Entry vector at said combinatorial sites, and
b) providing an Acceptor vector, wherein said Acceptor vector provides two combinatorial sites with associated recognition sites for at least one second type IIS restriction endonuclease of identical sequence to said two combinatorial sites present in the Entry vector.

In a second aspect, the invention provides a method of (sub)cloning at least one nucleic acid molecule of interest comprising
a) providing a (replicable) Donor vector comprising a nucleic acid molecule of interest to be transferred into an corresponding Acceptor vector,

wherein said Donor vector carries two recognition sites for an at least one first type IIS restriction endonuclease and wherein said nucleic acid molecule of interest can be excised from the at least one Donor vector at two combinatorial sites with one (same) or more (different) cohesive ends that are formed by the at least one first type IIS restriction endonuclease,

wherein the two recognition sites of the at least one first type IIS restriction endonuclease are arranged in the Donor vector in such relation to the combinatorial sites that said combinatorial sites are positioned in between these two type IIS restriction endonuclease recognition sites, and

wherein the two combinatorial sites are identical in sequence to two combinatorial sites present in the corresponding Acceptor vector, which are associated with at least one recognition site(s) in the Acceptor vector that are positioned in between said combinatorial sites,
b) providing an Acceptor vector, into which the at least one nucleic acid molecule of interest is transferred from the at least one Donor vector carrying the at least one nucleic acid molecule of interest, wherein said Acceptor vector comprises at least one recognition site for at least one second type IIS restriction endonuclease, and wherein said Acceptor vector provides two combinatorial sites identical to the two combinatorial sites present in the Donor vector.

In a third aspect, the invention provides a (replicable) Entry vector (cloning vector) into which the at least one nucleic acid molecule of interest is to be inserted,

wherein the at least one Entry vector carries two recognition sites for an at least one first type IIS restriction endonuclease and wherein said at least one nucleic acid molecule of interest can be excised from the at least one Entry vector at combinatorial sites with one (same) or more (different) cohesive ends that are formed by the at least one first type IIS restriction endonuclease,

wherein the two recognition sites of the at least first type IIS restriction endonuclease are arranged in the Entry vector in such relation to the combinatorial sites that said combinatorial sites are positioned in between these two type IIS restriction endonuclease recognition sites, and

wherein the Entry vector further comprises two recognition sites of an at least one third type IIS restriction endonuclease, wherein these two recognition sites of the at least one third type IIS restriction endonucleases are arranged such in the Entry vector that the one or two recognition sites of the third type IIS restrictions endonuclease are positioned in between the two recognition sites of the at least one first type IIS restriction endonuclease.

In a fourth aspect, the invention provides a nucleic acid cloning kit comprising
a) a (replicable) Entry vector into which the at least one nucleic acid molecule of interest is to be inserted, wherein the at least one Entry vector carries two recognition sites for a at least one first type IIS restriction endonuclease and wherein said at least one nucleic acid molecule of interest can be excised from the at least one Entry vector at two combinatorial sites with one (same) or more (different) cohesive ends that are formed by the at least one first type IIS restriction endonuclease, and
b) at least one Acceptor vector, into which the at least one nucleic acid molecule of interest can be transferred from the at least one Entry vector carrying the at least one nucleic acid molecule of interest, wherein said Acceptor vector comprises at least one recognition site for a second type IIS restriction endonuclease, and wherein said Acceptor vector provides combinatorial sites identical to the two combinatorial sites present in the Entry vector.

In a fifth aspect, the invention provides a (replicable) Entry vector (cloning vector) into which the at least one nucleic acid molecule of interest is to be inserted,

wherein the at least one Entry vector carries two recognition sites for an at least one first type IIS restriction endonuclease and wherein said at least one nucleic acid molecule of interest can be excised from the at least one Entry vector at combinatorial sites with one (same) or more (different) cohesive ends that are formed by the at least one first type IIS restriction endonuclease,

wherein the two recognition sites of the at least first type IIS restriction endonuclease are arranged in the Entry vector in such relation to the combinatorial sites that said combinatorial sites are positioned in between these two type IIS restriction endonuclease recognition sites, and

wherein the Entry vector further comprises two recognition sites of an at least one third type IIS restriction endonuclease, wherein these two recognition sites of the at least one third type IIS restriction endonucleases are arranged such in the Entry vector that the one or two recognition sites of the third type IIS restrictions endonuclease are positioned in between the two recognition sites of the at least one first type IIS restriction endonuclease.

In a sixth aspect, the invention provides a (replicable) Donor vector comprising a nucleic acid molecule of interest to be transferred into a corresponding Acceptor vector,

wherein said Donor vector carries two recognition sites for an at least one first type IIS restriction endonuclease and wherein said nucleic acid molecule of interest can be excised from the at least one Donor vector at two combinatorial sites with one (same) or more (different) cohesive ends that are formed by the at least one first type IIS restriction endonuclease,

wherein the two recognition sites of the at least one first type IIS restriction endonuclease are arranged in the Donor vector in such relation to the combinatorial sites that said combinatorial sites are positioned in between these two type IIS restriction endonuclease recognition sites, and

wherein the two combinatorial sites are identical in sequence to the two combinatorial sites present in the corresponding Acceptor vector, which are associated with at least one recognition site(s) in the Acceptor vector that are positioned in between said combinatorial sites.

The invention also provides in a seventh aspect a reaction mixture containing at least 2 nucleic acid molecules derived from different plasmids and carrying compatible cohesive ends that were generated by at least one type IIS restriction endonuclease and that are able to ligate to create a circular nucleic acid molecule that at least at one ligated site cannot be re-cut by said type IIS restriction endonuclease(s).

In an eight aspect the invention provides a method of (sub)cloning at least one nucleic acid molecule of interest from at least one replicable Entry vector into an Acceptor vector,

wherein the nucleic acid of interest is to be inserted into the at least one (replicable) Entry vector,

wherein the at least one Entry vector carries two recognition sites for at least one first type IIS and/or type IIS like restriction endonuclease and

wherein said at least one nucleic acid molecule of interest can be excised from the at least one Entry vector at two combinatorial sites with one (same) or more (different) cohesive ends that are formed by the at least one first type IIS and/or type IIS like restriction endonuclease,

the method comprising:

providing an Acceptor vector, into which the at least one nucleic acid molecule of interest is transferred from said at least one Entry vector carrying the at least one nucleic acid molecule of interest, wherein said Acceptor vector comprises at least one recognition site for at least one second type IIS restriction endonuclease and/or a recognition site for a second type IIS like restriction endonuclease, and wherein said Acceptor vector is adapted to provide two combinatorial sites identical to the two combinatorial sites present in the Entry vector.

In a ninth aspect the invention provide for a method of (sub)cloning at least one nucleic acid molecule of interest from an at least one (replicable) Entry vector into an Acceptor vector,

wherein the nucleic acid of interest is to be inserted into the at least one (replicable) Entry vector,

wherein the at least one Entry vector carries two combinatorial sites with associated recognition sites for at least one first type IIS and/or type IIS like restriction endonuclease,

and wherein said at least one nucleic acid molecule of interest can be excised from the at least one Entry vector at said combinatorial sites,

said method comprising

providing an Acceptor vector into which the at least one nucleic acid molecule of interest is transferred from said at least one Entry vector carrying the at least one nucleic acid molecule of interest, wherein said Acceptor vector is adapted to provide two combinatorial sites with associated recognition sites for at least one second type IIS restriction endonuclease of identical sequence to said two combinatorial sites present in the Entry vector or the Acceptor vector is adapted to provide two combinatorial sites with associated recognition sites for at least one type IIS like restriction endonuclease of identical sequence to said two combinatorial sites present in the Entry vector or the Acceptor vector is adapted to provide two combinatorial sites with associated recognition sites of both type IIS and type IIS like restriction endonucleases.

In a tenth aspect the invention provides for a method of (sub)cloning at least one nucleic acid molecule of interest from a replicable Donor vector into an Acceptor vector,

said Donor vector comprising the nucleic acid molecule of interest to be transferred into the Acceptor vector,

wherein said Donor vector carries two recognition sites for an at least one first type IIS and/or type IIS like restriction endonuclease and wherein said nucleic acid molecule of interest can be excised from the at least one Donor vector at two combinatorial sites with one (same) or more (different) cohesive ends that are formed by the at least one first type IIS restriction endonuclease,

wherein the two recognition sites of the at least one first type IIS restriction endonuclease are arranged in the Donor vector in such relation to the combinatorial sites that said combinatorial sites are positioned in between these two type IIS restriction endonuclease recognition sites, and

wherein the two combinatorial sites are identical in sequence to two combinatorial sites present in the corresponding Acceptor vector, which are associated with at least one recognition site(s) in the Acceptor vector that are positioned in between said combinatorial sites,

said method comprising

providing the Acceptor vector, into which the at least one nucleic acid molecule of interest is transferred from the at least one Donor vector carrying the at least one nucleic acid molecule of interest, wherein said Acceptor vector comprises at least one recognition site for at least one second type IIS restriction endonuclease or at least one recognition site for at least one type IIS like restriction endonuclease, and wherein said Acceptor vector is adapted to provide two combinatorial sites identical to the two combinatorial sites present in the Donor vector.

In an eleventh aspect the invention provides a method of (sub)cloning at least one nucleic acid molecule of interest from at least one replicable Entry vector into an Acceptor vector,

wherein the nucleic acid molecule of interest is to be inserted into the at least one (replicable) Entry vector,

wherein the at least one Entry vector carries two recognition sites for at least one first type IIS or type IIS like restriction endonuclease and

wherein said at least one nucleic acid molecule of interest can be excised from the at least one Entry vector at two combinatorial sites with one (same) or more (different) cohesive ends that are formed by the at least one first type IIS or type IIS like restriction endonuclease,

the method comprising:

providing an Acceptor vector, into which the at least one nucleic acid molecule of interest is transferred from said at least one Entry vector carrying the at least one nucleic acid molecule of interest, wherein said Acceptor vector is linearized and provides overhangs of two combinatorial sites identical to the two combinatorial sites present in the Entry vector, and wherein said combinatorial sites comprise a non-palindromic nucleic acid sequence.

In yet a further aspect the invention provides a method of (sub)cloning at least one nucleic acid molecule of interest from a replicable Donor vector into an Acceptor vector,

said Donor vector comprising the nucleic acid molecule of interest to be transferred into the Acceptor vector,

wherein said Donor vector carries two recognition sites for an at least one first type IIS or type IIS like restriction endonuclease and wherein said nucleic acid molecule of interest can be excised from the at least one Donor vector at two combinatorial sites with one (same) or more (different) cohesive ends that are formed by the at least one first type IIS or type IIS like restriction endonuclease,

wherein the two recognition sites of the at least one first type IIS restriction endonuclease are arranged in the Donor vector in such relation to the combinatorial sites that said combinatorial sites are positioned in between these two type IIS restriction endonuclease recognition sites, and

wherein the two combinatorial sites are identical in sequence to two combinatorial sites present in the corresponding Acceptor vector,

said method comprising

providing the Acceptor vector, into which the at least one nucleic acid molecule of interest is transferred from the at least one Donor vector carrying the at least one nucleic acid molecule of interest, wherein said Acceptor vector is linearized and provides overhangs of two combinatorial sites identical to the two combinatorial sites present in the Donor vector and wherein said combinatorial sites comprise a non-palindromic nucleic acid sequence.

### DETAILED DESCRIPTION OF THE INVENTION

In a first step of a method of the invention, a target nucleic acid molecule is inserted into an Entry vector to create a Donor vector. A one-step method is provided to perform this insertion relying on type IIS restriction endonucleases or type IIS like restriction endonucleases. For this purpose, the target nucleic acid molecule is usually equipped at both ends with combinatorial sites by, e.g., PCR using dedicated primers (provision of the combinatorial sites is of course not necessary, if the target nucleic acid molecule, has, for example, by chance, already one or both combinatorial sites at its 3′ or 5′-end). A recognition site for a (first) type IIS restriction endonuclease is brought in operative linkage with said two combinatorial sites, for example, by using primers with accordingly designed 5′ appendages or by ligating an adapter oligonucleotide to the PCR product. Furthermore, combinatorial sites introduced at both ends of the nucleic acid molecule may be identical to the combinatorial sites that are present in the Entry vector (cf. FIG. 1). After cleavage with a type IIS restriction endonuclease, complementary cohesive ends are therefore generated in both the nucleic acid molecule and the Entry vector. These cohesive ends anneal in an oriented manner creating the Donor vector after ligation. Positioning of the recognition sequences of the used type IIS restriction endonuclease(s) leads to elimination of the recognition sequences from the resulting Donor vector. Therefore, cleavage of the nucleic acid molecule and the Entry vector and ligation of said recombined nucleic acid fragments to create a Donor vector can be performed efficiently in one step in one single reaction mixture.

Furthermore, methods are provided in the present invention that address the problem of “internal” (i.e. pre-existing recognition sites in regions of the target nucleic acid molecules such as genes not derived from the synthesis primers or vectors) type IIS restriction endonuclease recognition sites of the same type that have to be used in the initial and/or subsequent transfer reactions. One alternative method to create a Donor vector does not rely on the methods of the invention but simply consists of a blunt end ligation of the nucleic acid molecule (PCR fragment) with a pre-cut blunt end Entry vector. In this case, the combinatorial sites are preferentially added to the nucleic acid molecule, preferentially via PCR primers, and are brought into operative linkage with a type IIS restriction endonuclease recognition site, that is present at the ends of the pre-cut Entry vector, through the ligation reaction only (cf. FIG. 8). Nucleic acid molecules of interest, after being transferred into Donor vectors should preferentially be sequenced for verification of their nucleic acid sequence, particularly when PCR had been involved during cloning of the gene and/or subsequent equipping the nucleic acid molecule with the combinatorial sites.

In a second step of a method of the invention, one or more nucleic acid molecule(s) of interest are excised from the Donor vector by a second type IIS restriction endonuclease or a second type IIS like restriction endonuclease and are recombined via compatible combinatorial sites with an Acceptor vector in a directed manner in order to create a Destination vector. Alternatively, individual excised nucleic acid molecules are intermediately assembled in respective Entry vectors in a certain combination prior to be transferred into an Acceptor vector to create a Destination vector. The dedicated positioning of type IIS restriction endonuclease recognition sites and of the combinatorial sites ensures unique compatibility of nucleic acid molecules resulting in a directed assembly of individual nucleic acid molecules so that type IIS restriction endonuclease recognition sites are eliminated from the desired intermediate or final vector product (Entry or Destination vector, respectively) after ligation. This enables assembly of two or more nucleic acid molecules in a single reaction without the need of intermediate purification steps after cleavage and prior to ligation (i.e. cleavage and ligation are performed in the same reaction mixture). Selection of assembled nucleic acid molecules (Destination vectors) may be facilitated by using Donor and Acceptor vectors with different selectable markers and using a reporter gene in the Acceptor vector that is eliminated by insertion of the nucleic acid molecule(s). When relying on the use of type IIS restriction endonucleases, only the sequences of the cohesive ends (combinatorial sites)—but not the sequences of the recognition sites—appear in the final nucleic acid (Destination vector). In the present invention, these sequences are usually 1 to 5 bases in length. Depending on the type IIS enzyme used blunt ends can, however, also be generated. The remaining sequences of the cohesive ends are minimal cloning associated changes of the initial sequences of the nucleic acid molecules as compared to, for example, natural recombination sites (e.g. attB or loxP, which are, 25 or 34 bases in length, respectively) which will be present using cloning systems such as GATEWAY™. This reduction of unrelated sequences achieved in the present invention minimizes the risk of changing properties of nucleic acid molecules such as gene(s) or gene product(s) to be analyzed.

The high degree of versatility and simplicity of the methods and products of the invention enables straightforward systematic recombination and, for example, thus efficient studies of almost authentic target nucleic acid molecules such as genes in various genetic contexts. Moreover, de novo vector construction is reduced to combination of nucleic acid molecules exhibiting position determining specific combinatorial sites that may be cleaved by at least one type IIS restriction endonuclease to generate compatible cohesive ends for directed assembly of multiple nucleic acid molecules in a single reaction.

The invention will be better understood from the following description and with reference to the following definitions.

### DEFINITIONS

### Acceptor Vector

An Acceptor vector is a vector having two (2) divergently oriented type IIS restriction endonuclease recognition sites defining combinatorial sites that are compatible with combinatorial sites defined by the convergently oriented type IIS restriction endonuclease recognition sites present in Entry and/or Donor vector(s) thereby enabling the oriented insertion of one or more nucleic acid molecules provided by Entry and/or Donor vectors. This (divergent) positioning of type IIS recognition site(s) leads to their elimination from the resulting chimeric vector.

An Acceptor vector can be provided in the present invention, when used for reaction with a Donor vector, either in circularized or linearized form. When provided in linearized form, the Acceptor vector may have been opened and linearized in any suitable way as long as the linearized Acceptor vector is capable of ultimately providing the desired (free) cohesive ends. In one illustrative example, the Acceptor vector can be opened/linearized by cleavage of any restriction endonuclease, for example any regular type IIP restriction endonuclease, at an arbitrary position between the two at least one second (divergent) type IIS restriction endonuclease recognition sites. In this approach the desired/necessary cohesive ends for uptake of the nucleic acid molecule from the Donor vector will be created by the at least one second type IIS or type IIS like restriction endonuclease during the reaction with the Donor vector. In another illustrative example, the Acceptor vector can be opened/linearized by cleavage of the at least one second type IIS restriction endonuclease. In this approach the cohesive ends of the Acceptor vector comprise the combinatorial sites and are available prior to the reaction with the Donor vector for uptake of the nucleic acid molecule from the Donor vector after excision with the at least one first type IIS restriction endonuclease.

### Adapter Oligonucleotide

Type IIS restriction endonucleases cleave the nucleic acid remote from the recognition site. Thus, if the recognition site is positioned at the extreme ends of an annealed pair of two at least partially complementary synthetic oligonucleotides or, alternatively, at the end of the stem of a monomeric oligonucleotide forming a stem-loop and if such synthetic recognition site is ligated to the ends of a target nucleic acid molecule, cohesive ends may be generated in said target nucleic acid molecule by cleavage of a type IIS restriction endonuclease. These cohesive ends may be of predestined/predefined sequence if the target nucleic acid molecule had been equipped with combinatorial sites, or at least with a part of the combinatorial sites (in the latter case the residual part may then be provided by the adapter oligonucleotide), by, e.g., PCR. These combinatorial sites (or parts thereof) may, however, also be attached to the nucleic acid molecule by other methods well known to the person skilled in the art. Thus, the term “adapter oligonucleotide” denotes any nucleic acid comprising a sequence that forms a recognition site for a type IIS restriction endonuclease positioned so that said type IIS restriction endonuclease is at least in part not able to cleave the adapter molecule but will cleave at least one strand of a foreign nucleic acid molecule that has been ligated to the adapter molecule.

### Combinatorial Site

The term “combinatorial site” as used herein is a specific (usually predetermined) nucleic acid sequence that forms a specific cohesive end after cleavage with a type IIS restriction endonuclease. The term “combinatorial site” thus denotes any suitable nucleic acid sequence that is the cleavage target of a type IIS restriction endonuclease (or of a type IIS like restriction endonuclease in certain embodiments as explained below) for recombination with a further compatible combinatorial site. The sequence of the combinatorial site defines the position and/or orientation of the nucleic acid molecule in the final assembly. This is to be considered in the design of a strategy where more than one nucleic acid molecule is, for example, transferred for the de novo construction of vectors. In the situation where only one defined nucleic acid molecule of interest is brought into different but defined genetic surroundings by sub-cloning the nucleic acid molecule into respective Acceptor vectors carrying such genetic surroundings, an Entry vector is chosen that has convergent recognition sites defining combinatorial sites that are compatible with the combinatorial sites present in all Acceptor vectors carrying the genetic surroundings of interest. Or, taking the opposite approach, Acceptor vectors are provided that have identical combinatorial sites in operative linkage with a series of different genetic surroundings that are desired to be evaluated in the context of the nucleic acid molecule of interest. An illustrative example is the provision of different affinity tags that are evaluated in the context of a gene to be expressed. In contrast to the type IIS restriction endonuclease recognition sequences that will be preferentially eliminated from the final assembly in the sub-cloning process of the invention, the combinatorial sites remain in the final assembly. As an advantage towards the Gateway™ methodology, the sequence of the combinatorial sites used in the present invention can be freely chosen. This has the advantage that functional elements can be included in the combinatorial sites so that they do not necessarily imply a foreign function or alteration like in Gateway™. An illustrative example is that an ATG start codon can easily constitute a combinatorial site for a type IIS restriction endonuclease such as LguI creating cohesive ends of 3 bases in length which can be exploited to clone genes in Destination vectors carrying authentic N-terminal ends.

The term “convergent” type IIS restriction endonuclease recognition site(s) as used herein means that at least two (2) recognition sites are arranged such in relation to one or more of the respective combinatorial site(s) that said combinatorial site(s) are arranged in between said recognition sites (cf. the Donor vector of FIG. 1C and FIG. 2A, in which the combinatorial sites “ATG” and GGG (FIG. 1C) and “AATG” and “GGGA” (FIG. 2A) are arranged in between the two associated Esp3I recognition sites).

The term “divergent” type IIS restriction endonuclease recognition sites as used herein means that two (2) or more combinatorial sites are arranged such in relation to one or more of their associated type IIS restriction endonuclease recognition site(s) that the type IIS endonuclease recognition site(s) are arranged in between said combinatorial sites (see for example, FIG. 1C, where two SapI recognition sites are arranged in the Entry vector in between the “ATG” and “CCC” combinatorial sites).

In this context, it is noted that the terms “convergently oriented”, “convergent orientation”, “divergently oriented”, “divergent orientation” when used here in connection with type IIS restriction endonucleases are only applicable for type IIS restriction endonucleases that cleave a nucleic acid molecule only in one direction, either in 5′- or 3′ direction. These terms are not applicable when those “special type” type IIS restriction endonucleases that cleave the target DNA at the same time at 2 specific sites in both 5′ and 3′ direction from the recognition site are used herein.

### Destination Vector

A “Destination vector” as used herein is a vector obtained herein as result of a transfer reaction between a Donor vector and an Acceptor vector. A destination vector contains one or more nucleic acid molecules that cannot (any longer) be excised by means of a type IIS restriction endonuclease nor is the destination vector designed for or capable of inserting further nucleic acid molecules of interest like for the purpose of this invention via type IIS restriction endonucleases. Accordingly, a Destination vector typically does not comprise any type IIS restriction endonuclease recognition sites at all to be used for the purpose of this invention but only the fixed combinatorial sites (see the Destination vector of FIG. 2B which only comprises the nucleic acid molecule of interest arranged in context with the “AATG” and “GGGA” combinatorial site sequences).

### Donor Vector

A “Donor vector” as used herein is a nucleic acid molecule such as a plasmid DNA with one or more inserted nucleic acid molecules that may be excised via convergently oriented type IIS endonuclease recognition sites at combinatorial sites compatible to the combinatorial sites present in an Acceptor or Entry vector.

### Entry Vector

An “Entry vector” as used herein is a nucleic acid molecule such as a plasmid DNA designed for the insertion of one or more target nucleic acid molecules. For this purpose an Entry vector typically comprises divergently oriented type IIS recognition sites (see the Entry vector of FIG. 1C in which the two SapI recognition sites are divergently arranged). Another feature of an Entry vector is that it additionally comprises at least 2 convergently arranged type IIS recognition sites (typically, these convergently arranged type IIS recognition sites differ from the divergently arranged type IIS recognition sites) for excision of the one or more target nucleic acid molecule(s) (after being inserted) for transfer of the target nucleic acid molecule(s) into an Acceptor or an other Entry vector (see, for example, the Entry vector 3 shown in FIG. 4A and FIG. 4B wherein the SapI recognition sites, the Entry vector 1 shown in FIG. 4A wherein the Esp3I recognition sites or Entry vector 4 shown in FIG. 4B wherein the BsaI recognition sites represent such convergently oriented type IIS restriction endonuclease recognition sites). In this regard it should be noted that if a nucleic acid molecule is inserted in an Entry vector together with 2 divergently oriented type IIS restriction endonuclease recognition sites on the same nucleic acid fragment then a new (further) Entry vector is generated that is capable for the uptake of a further nucleic acid molecule (see FIG. 4A and the respective description thereof, wherein the BsaI recognition sites of Entry vector 1 represent such divergently oriented type IIS restriction endonuclease recognition sites). This strategy is useful for the sequential assembly of multiple nucleic acid molecules. It should be noted that a typical Entry vector carries the characteristics of both a Donor vector and an Acceptor vector.

It should also be noted here that an Entry vector can be provided in the present invention, when used for reaction with a PCR product (or with a Donor vector), either in circularized or linearized form. When provided in linearized form, the Entry vector may have been opened and linearized in any suitable way as long as the linearized Entry vector is capable of ultimately providing the desired (free) cohesive ends. In one illustrative example, the Entry vector can be opened/linearized by cleavage of any restriction endonuclease, for example any regular type IIP restriction endonuclease, at an arbitrary position between two of the at least one third (divergent) type IIS restriction endonuclease recognition sites. In this approach the necessary cohesive ends for uptake of the nucleic acid molecule from the Donor vector or PCR product will be created by the at least one third type IIS or type IIS like restriction endonuclease during the reaction with the Donor vector or PCR fragment. In another illustrative example the Entry vector can be opened/linearized by cleavage of the at least one third type IIS restriction endonuclease so that the cohesive ends of the Acceptor vector comprise the combinatorial sites and are available prior to the reaction with the Donor vector or PCR fragment for uptake of the nucleic acid molecule from the Donor vector or PCR fragment after cleavage with the at least one first type IIS restriction endonuclease.

### Nucleic Acid Molecule

The term “nucleic acid molecule” or “nucleic acid molecule of interest” or “target nucleic acid” denotes any functional nucleic acid sequence element that may be recombined with other elements to create new nucleic acid molecules such as plasmids, expression vectors, viruses, etc by application of methods of the present invention. The nucleic acid molecule of interest will generally be engineered to be equipped at both of its termini with combinatorial sites. Illustrative examples for such nucleic acid molecules are, without limitation, a structural (target) gene to be expressed, a promoter, a promoter regulating site (operator or enhancer), a translation initiation site, a signal sequence for secretion or other subcellular localization, a terminator for transcription, a polyadenylation signal, a C-terminal affinity tag (for example a STREP-TAG®, His-tag, Flag-tag, myc-tag, HA-tag, GST-tag, thioredoxin-tag, SNAP-tag and the like), an N-terminal affinity tag, a reporter gene (fluorescent protein, enzyme, and the like), a protease cleavage site, an origin of replication, a selectable marker, and the like. The nucleic acid of interest may also be an assembly of genes to be expressed or any other modular assembly of genes, for example an expression cassette that comprises one or more regulatory sequences and target genes which are modularly assembled in a polycistronic operon and placed under the functional control of such regulatory sequences.

### Type IIS Like Restriction Endonuclease

The use of type IIS like restriction endonucleases as defined herein is also contemplated in the present invention and they can be used in the present invention in a similar manner as type IIS restriction endonucleases, meaning whenever a type IIS restriction endonuclease is used, it can be replaced by a type IIS like restriction endonuclease. This means that the present invention also comprises Entry and Acceptor vectors in which type IIS and type IIS like recognition sites are mixed to create combinatorial sites. For example, an Acceptor vector can comprise one recognition site for a second type IIS restriction endonuclease and one recognition site for a second type IIS like restriction endonuclease to create the overhangs at combinatorial sites for uptake of a nucleic acid molecule excised from a Donor vector. Likewise, also an Entry vector can comprise one recognition site for a first type IIS restriction endonuclease combined with a first type IIS like restriction endonuclease for excision of the nucleic acid molecule at combinatorial sites.

The type IIS like restriction endonucleases include enzymes such as AasI, AdeI, BglI, Bme1390I, BseLI, BsiYI, BstXI, CaiI, DmIII, DrdI, Eam1105I, EcoNI, Fnu4HI, HpyF10VI, MwoI, PflMI, PsyI, SatI, ScrFI, SfiI, TaaI, Tsp4CI, Tth111I, Van91I, XagI. The type IIS like restriction endonucleases have a split recognition site wherein for each enzyme the defined elements are separated by an arbitrary sequence of a defined length and wherein the DNA strands are cleaved within the arbitrary sequence to create overhangs. Thus the overhangs to be generated can be freely chosen by placing a corresponding sequence between the defined elements. Such enzymes may be useful—also in a highly parallel manner—to generate linearized Acceptor vector like DNA that is then able to ligate with a nucleic acid molecule excised from a Donor vector at combinatorial sites. It is also possible to use type IIS like restriction endonucleases in circularized Acceptor vectors or Entry vectors into which one or more nucleic acid molecules of interest are transferred. In either case, meaning if type IIS like restriction endonucleases are used to replace type IIS restriction endonucleases in Acceptor vectors (at least) one or two IIS like restriction endonuclease are present in order to generate (the overhangs of) combinatorial sites via which the ligation of a nucleic acid of interest into an Acceptor vector occurs.

### Type IIS Restriction Endonuclease

The term “type IIS restriction endonucleases” is used herein in its usual meaning as explained by Szybalski et al., 1991, Gene 100, pages 13-26 for example to refer to the class of endonucleases that—unlike the most characterized and frequently used type IIP restriction enzymes that cleave inside their recognition sequence—cleave nucleic acid molecules at a specified position up to, for example, 20 bases remote from the recognition site. Illustrative examples for type IIS restriction endonucleases with known recognition sites that can be used in the present invention include, but are not limited to AarI, AceIII, AloI, Alw26I, BaeI, Bbr7I, BbvI, BbvII, BccI, Bce83I, BceAI, BcefI, BcgI, BciVI, BfiI, BfuI, BinI, BpiI, BsaI, BsaXI, BscAI, BseMI, BseMII, BseRI, BseXI, BsgI, BsmI, BsmAI, BsmFI, Bsp24I, BspCNI, BspMI, BspPI, BsrI, BsrDI, BstF5I, BtsI, CjeI, CjePI, EciI, Eco31I, Eco57I, Eco57MI, Esp3I, FaII, FauI, FokI, GsuI, HaeIV, HgaI, Hin4I, HphI, HpyAV, Ksp632I, LguI, MboII, MlyI, MmeI, MnII, PleI, PpiI, PsrI, RleAI, SapI, SchI, SfaNI, SspD5I, Sth132I, StsI, TaqII, TspDTI, TspGWI, or Tth111II.

### DESCRIPTION OF EXEMPLARY EMBODIMENTS

The invention is based, in part, on the finding of the present inventors to systematically position recognition sites of restriction endonucleases known as type IIS restriction endonucleases or type IIS like restriction endonucleases in a new manner in cloning vectors. As mentioned above, examples for suitable type IIS restriction endonucleases with known recognition sites include, but are not limited to AarI, AceIII, AloI, Alw26I, BaeI, Bbr7I, BbvI, BbvII, BccI, Bce83I, BceAI, BcefI, BcgI, BciVI, BfiI, BfuI, BinI, BpiI, BsaI, BsaXI, BscAI, BseMI, BseMII, BseRI, BseXI, BsgI, BsmI, BsmAI, BsmFI, Bsp24I, BspCNI, BspMI, BspPI, BsrI, BsrDI, BstF5I, BtsI, CjeI, CjePI, EciI, Eco31I, Eco57I, Eco57MI, Esp3I, FalI, FauI, FokI, GsuI, HaeIV, HgaI, Hin4I, HphI, HpyAV, Ksp632I, LguI, MboII, MlyI, MmeI, MnII, PleI, PpiI, PsrI, RleAI, SapI, SchI, SfaNI, SspD5I, Sth132I, StsI, TaqII, TspDTI, TspGWI, and Tth111II. Type IIS restriction endonucleases and various uses thereof are summarized by Szybalski et al., 1991, Gene 100, pages 13-26. Examples of suitable type IIS like restriction endonucleases include, but are not limited to, AasI, AdeI, BglI, Bme1390I, BseLI, BsiYI, BstXI, CaiI, DraIII, DrdI, Eam1105I, EcoNI, Fnu4HI, HpyF10VI, MwoI, PflMI, PsyI, SatI, ScrFI, SfiI, TaaI, Tsp4CI, Tth111I, Van91I, and XagI.

The invention is secondly based, in part, on the finding of the inventors to use certain orientations of individual restriction recognition sites relative to the nucleic acid molecule which is located between these sites. This orientation permits amongst others (i) the generation of certain pairs of compatible combinatorial sites between individual molecules for directed assembly, (ii) the elimination or retention of the type IIS restriction enzyme recognition sites according to the needs of downstream applications and (iii) the head-to-head combination of specific recognition sites in order to vary the length of the cohesive ends to be generated at specific combinatorial sites.

The invention is thirdly based, in part, on the finding of the inventors to use distinct synthetic adapter oligonucleotides which contain the recognition sites of type IIS restriction endonucleases. These oligonucleotides are readily fused to the end(s) of individual nucleic acid fragments comprising a nucleic acid molecule in order to introduce type IIS restriction endonuclease recognition sites for generation of cohesive ends that are composed at least in part of sequences derived from the nucleic acid molecule and not from the adapter oligonucleotide. The use of such adapter oligonucleotides has the following advantages. It permits (i) a significant reduction of cloning-associated costs by reducing primer syntheses efforts in order to create cohesive ends at specific combinatorial sites, which are necessarily attached to cloning primers in all previously applied techniques, it allows (ii) facilitated generation of chimeric DNAs comprising a multitude of directed assembled nucleic acid molecules and finally it allows the (iii) facilitated generation of site-directed mutagenesis within individual nucleic acid molecules which can be used to edit genetic information during the cloning procedure (e.g. the elimination of disturbing cleavage sites or undesirable rare codons is readily achieved). Alternatively to bringing a type IIS restriction endonuclease recognition site into an operative linkage with a combinatorial site via an adapter molecule, it is also possible to ligate the blunt end PCR product with an opened vector fragment carrying the recognition sites closely at the terminal blunt ends.

Unlike the most characterized and frequently used type IIP restriction endonucleases that cleave inside their recognition sequence, type IIS cleave DNA at a specified position up to 20 bases remote from the recognition site (see Szybalski et al., 1991, Gene, supra, for example). Depending on the type IIS restriction enzyme, DNA is either cleaved to create blunt ends if both DNA strands are cleaved at the same distance relative to the recognition sequence or to create cohesive ends if both strands are cleaved at different distances relative to the recognition sequence. Cohesive ends created by type IIS restriction enzymes are typically between 1 and 5 nucleotides in length and are created carrying the nucleotide sequence specified by the sequence residing at that position in the substrate DNA. Further, special type IIS restriction endonucleases are known that cleave the target DNA at the same time at 2 specific sites in both 5′ and 3′ direction from the recognition site. Examples for such type IIS restriction sites are, but not limited to, AjuI, AlfI, AloI, BaeI, BcgI, BdaI, BplI, CspCI, FalI, Hin4I, PpiI, PsrI, TstI. Such special type IIS restriction endonucleases are able to e.g. open an Acceptor vector at 2 combinatorial sites on behalf on one recognition site only while the use of normal type IIS restriction endonucleases would require 2 divergently oriented recognition sites.

It was found to the surprise of the inventors that type IIS restriction enzymes can be efficiently used for a cloning system that offers the advantages of the GATEWAY™ system, but at the same time additionally allows a one-step procedure/one tube reaction for subcloning of (target) nucleic acid molecules, without being restricted to the incorporation or appendage of major DNA segments to the nucleic acid molecule in the final Destination vector. One single type IIS restriction endonuclease is able to generate a multitude of different cohesive ends by cleaving at the predefined combinatorial sites (the equivalent to the recombination sites in the GATEWAY™ system). Thus, in principle, if, e.g., a 4 base cohesive end is created, one single type IIS restriction enzyme of such functionality is able to produce 4⁴=256 different cohesive ends which may be used to assemble a multitude of nucleic acid molecules in a predefined oriented manner.

In one embodiment, the present invention provides methods to synthesize new plasmids by combining two or more (i.e. a plurality) nucleic acid molecules in a predefined manner. These methods provide as new plasmid (i) an at least one (replicable) Entry vector into which the at least one nucleic acid molecule is to be inserted, wherein the at least one Entry vector carries two recognition sites for at least one first type IIS restriction endonuclease and wherein said at least one nucleic acid molecule can be excised from the at least one Entry or Donor vector at combinatorial sites with one (same) or more (different) cohesive ends that are formed by the at least one first type IIS restriction endonuclease. These methods also provide as new plasmid (ii) an Acceptor vector, into which the at least one nucleic acid molecule can be transferred from the at least one Entry or Donor vector carrying the at least one nucleic acid molecule, wherein said Acceptor vector comprises at least one recognition site for at least one second type IIS restriction endonuclease and wherein said Acceptor vector provides combinatorial sites identical to the combinatorial sites present in the Entry or Donor vector.

In the first step, the nucleic acid molecule of interest is inserted into an Entry vector to thereby create a Donor vector. This insertion is performed in such a way that the nucleic acid molecule of interest is placed between combinatorial sites and convergent recognition sites of one or more type IIS restriction endonucleases so that upon cleavage with corresponding type IIS restriction endonucleases said nucleic acid molecule may be excised with cohesive ends formed by the sequences of the combinatorial sites. These specific combinatorial sites are advantageously asymmetric (non-palindromic) and different for each junction to be formed. This enables directed assembly and prevents non-desired side reactions such as concatamer formation in the subsequent recombination and ligation reaction that are carried out for multimerization and/or for insertion in an Acceptor vector via compatible combinatorial sites. In a further advantageous embodiment, the nucleic acid molecule(s) is positioned close/adjacent to the combinatorial sites defined by the convergent type IIS restriction endonuclease recognition sites in the Donor vector to avoid carrying along superfluous extra nucleic acid sequences (in some cases, like e.g. for the fusion of nucleic acid molecules, it may however be desirable to deliberately add bases to one end of a nucleic acid molecule which may serve as linker element for example; cf. FIG. 9). Said insertion of nucleic acid molecules into an Entry vector may be easily performed in a single reaction, including ligation in the presence of the type IIS restriction endonuclease, with methods that are disclosed by U.S. Pat. No. 6,261,797. As an improvement relative to the methods of U.S. Pat. No. 6,261,797 it was unexpectedly found here that releasable primers described in U.S. Pat. No. 6,261,797 can be replaced by non-releasable primers. Such non-releasable primers have combinatorial sites or at least a part thereof at their 5′ end but lack the recognition site of the type IIS restriction endonuclease. The combinatorial sites are fused to the nucleic acid molecule by PCR. The restriction endonuclease recognition site is provided in this embodiment by a separate adapter oligonucleotide that is ligated to the PCR product. After cleavage, the target DNA is cut precisely at the predetermined specific combinatorial sites to create the desired cohesive ends for subsequent directed ligation with the opened Entry vector (see FIG. 1).

Alternatively to ligating an adapter oligonucleotide to both ends of the PCR product(s), the PCR product(s) may be inserted, for example, via blunt ends, into linearized adapter plasmid DNA that provides convergent recognition sites of the type IIS restriction endonuclease(s). The thereby created circular plasmid DNA is the equivalent of a Donor vector that enables the transfer of the PCR product(s) into an Entry vector by a reaction that is similar to the one depicted in FIG. 2, the only difference being that the Acceptor vector of FIG. 2B is replaced by an Entry vector. Alternatively, the adapter plasmid DNA may be used directly as Entry vector which after insertion of the nucleic acid molecule of interest is capable to act as Donor vector to transfer this nucleic acid molecule (PCR product) into an Acceptor vector or a multitude of Acceptor vectors. In this case, the adapter plasmid should be designed to carry appropriate convergent type II S restriction endonuclease recognition sites for appropriate cleavage of the combinatorial sites. Said combinatorial sites necessary for the transfer reactions are preferentially attached to the nucleic acid molecule prior to insertion (e.g. via PCR as described in FIG. 8). More details of using Entry vectors with divergent type IIS restriction endonucleases cutting blunt ends are disclosed in the description of the embodiment of FIG. 8.

These approaches have the advantage that the adapter oligonucleotide or adapter plasmid part containing the type IIS restriction endonuclease recognition sequence does not have to be integrated at each primer anew for each new generation of a desired target nucleic acid molecule. Thereby oligonucleotide synthesis costs are saved. These approaches also reduce the risk of non-specific PCR product formation because these type IIS restriction endonuclease recognition sequences have no complementary site in the template DNA. An even more important advantage is related to the use of inhibitory nucleotide base analogues to prevent cleavage at internal sites. The method described in U.S. Pat. No. 6,261,797, pages 9 to 11, has several limitations since only one strand of the recognition site in the final PCR product is created by the primer while the complementary strand is synthesized during PCR. By so doing, inhibitory base analogues are potentially incorporated which may prevent the desired cleavage at the combinatorial sites. With the adapter oligonucleotide or the aforementioned linearized adapter plasmid methodologies used in the present invention, both strands of the asymmetric recognition sequence are provided by the synthetic oligonucleotide(s) or by the adapter plasmid, respectively. For this reason, the PCR strategy using inhibitory base analogues to prevent cutting at internal sites can be performed with any type IIS restriction endonuclease and without any special precautions for directed cloning of the PCR product by means of the specific combinatorial sites into the Entry vector to create a Donor vector. It is obvious to the person skilled in the art that other methods than PCR may be used to equip the nucleic acid molecule with combinatorial sites or parts thereof, e.g. ligating a hybridized oligonucleotide carrying the combinatorial site. The method for Donor vector generation of the embodiment shown in FIG. 8 completely lacks the need for a restriction enzyme cleavage reaction and thereby totally circumvents the problem described above.

An illustrative example, without limitation, for one suitable way to create a Donor vector is as follows (see also FIG. 1):

1. Amplifying the nucleic acid molecule of interest via polymerase chain reaction (PCR) using a thermostable DNA polymerase, preferentially with proof-reading activity, and primer sequences that carry at the 5′ end combinatorial sites or a part thereof additionally to the sequence hybridizing to the nucleic acid molecule in the template DNA. The amplification is carried out using a reaction buffer suitable for the thermostable DNA polymerase and a nucleotide base mix (dNTP's) that is equipped with preferably at least one inhibitory nucleotide base analogue.

2. Mixing the PCR product (either purified or unpurified) with (i) an Entry vector that carries combinatorial sites compatible to the combinatorial sites from step 1 above and recognition sequences for one or more type IIS restriction endonucleases and with (ii) an adapter oligonucleotide. Preferably, the recognition sequences are positioned in the Entry vector in such a way that, after cleavage, they are removed as by-product and replaced by the PCR amplified nucleic acid molecule to create the Donor vector. It is also possible to have a marker in the by-product so that, after having performed the transfer reaction, bacterial clones carrying the Entry vector without inserted nucleic acid molecule can be distinguished from, for example, bacterial clones that carry the Donor vector. An example for such a suitable marker is the part of the lacZ gene encoding the alpha-peptide including promoter (lacP/Zα) which enables blue/white selection which is well known to person skilled in the art. Examples for other markers that could be used for the same purpose include, but are not limited to a suicide gene such as ccdB or a gene for a green or yellow fluorescent protein.

3. Adding the respective type IIS restriction endonuclease(s), ligase, polynucleotide kinase when non-phosphorylated PCR-primers and adapter oligonucleotides (or adapter plasmid) have been used, ATP, and buffer components and incubating the reaction mixture at a temperature at that the enzymes are active. Due to their specific and defined configuration all restriction endonuclease recognition sequences for the type IIS restriction endonucleases present in the reaction mixture have been removed from the Donor vector once this has formed. Thus, in contrast to the Entry vector, which may be permanently cleaved and religated, the Donor vector is a stable product in the reaction mixture, so that the reaction proceeds efficiently and is directed to give the desired Donor vector in good yield. The fact that the resulting Donor vector is precluded from the reaction because the reverse reaction is not possible due to the lack of the recognition sites of those type IIS restriction endonuclease(s) present in the reaction mixture is an advantage over the GATEWAY™ system. In the GATEWAY™ system an equilibrium forms between the vectors introduced into the reaction and the desired vector reaction products because the reverse reaction is possible as well thereby potentially leading to reduced Donor vector yield.

4. Transformation of host systems such as bacteria such as E. coli, (for example a mcrABC mutant without restriction system for nucleic acids carrying nucleotide base analogues), and selection of white clones on X-Gal containing plates. If a bacterial strain is used which carries the lac repressor gene, IPTG has also to be added to the plates.

5. Isolating of Donor vector plasmid DNA and sequencing of the inserted nucleic acid molecule for verification.

In the second step, a transfer reaction is performed to fuse the nucleic acid molecule of interest with other nucleic acid molecules and/or (finally) with an Acceptor vector. In an illustrative example to describe this approach, the nucleic acid molecule in the Donor vector is a (structural) gene that is to be fused with other nucleic acid molecules that enable expression of the (structural) gene as fusion with a purification tag at the C-terminal end. Thus the gene is to be fused at its 5′ end with a promoter/rbs (rbs=ribosomal binding site) sequence and at its 3′ end with a nucleotide sequence encoding the purification tag. In this example, this promoter/rbs sequence and the nucleotide sequence encoding the purification tag are provided by the Acceptor vector, pre-assembled with further nucleic acid molecules necessary for propagation of the plasmid in e.g. E. coli (e.g. selectable marker, origin of replication), and, carrying combinatorial sites 3′ to the promoter/rbs sequence and 5′ to the sequence encoding the purification tag. The transfer reaction thus comprises incubating the Donor vector and the Acceptor vector together with at least one type IIS restriction endonuclease that cuts both vectors at the combinatorial sites. Thereby, the gene is excised from the Donor vector and compatible cohesive ends are provided in the Acceptor vector so that both nucleic acid fragments may recombine and create a Destination vector after ligation (see also FIGS. 2A and 2B). A plurality/multitude of Acceptor vectors carrying identical combinatorial sites in combination with other functional or regulatory elements, e.g. elements for fusion of the gene with other tags or with other promoters and the like, can be provided so that the gene may be transferred in parallel into different genetic surroundings. Thus, the only element that has to be kept constant to enable subcloning of a gene into a multitude of different genetic surroundings provided by Acceptor vectors are the combinatorial sites which are cleaved by a type IIS restriction endonuclease to create compatible cohesive ends for directed assembly of nucleic acid molecules in the Destination vector. Recognition sequences of type IIS restriction endonuclease(s) are typically designed in the present invention in such a way that they are removed from the Destination vector upon formation. This arrangement optimizes the one-step reaction comprising the transfer of the nucleic acid molecule from the Donor vector into the Acceptor vector, thereby creating a Destination vector in the presence of type IIS restriction endonuclease and ligase because the Destination vector is the only stable product in the reaction mixture. Thus, after its formation, the Destination vector is not longer available for the reaction and shifts the equilibrium of the reaction towards formation of the Destination vector (see also FIGS. 1 and 2). When using a special type IIS restriction endonuclease like AjuI, that cleaves in both directions relative to the recognition site, already the integration of one such recognition site into the Acceptor vector is sufficient to create the specific cohesive ends for directed cloning of the nucleic acid molecule from the Donor vector into the Acceptor vector to create the Destination vector. An illustrative example for the use of the methods of the invention is a subcloning system for screening optimal purification tag:promoter (specific for different host organisms) combinations as outlined by FIG. 11.

Using one single type IIS restriction endonuclease for oriented assembly of a multitude of nucleic acid molecules is one presently preferred embodiment of the invention as this has the advantage to, for example, (i) reduce costs, (ii) reduce the risk of occurrence of “internal” restriction sites which may reduce subcloning efficiency and (iii) reduce the risk of experimental failures as the proper handling of one restriction endonuclease has to be learned by the novice researcher only. As, according to the invention, type IIS restriction endonuclease recognition sites are positioned in a way that they are removed from the desired product, a further presently preferred embodiment of the invention is that restriction and ligation is performed simultaneously in the reaction mixture.

In a further presently preferred embodiment, Donor vector and Acceptor vector—present in a reaction mixture that contains one or more type IIS restriction endonucleases and ligase—each carry different selectable markers so that, after transformation, Acceptor and Destination vectors can be selected without selecting clones carrying a Donor vector. In this context it should be noted that creating Acceptor vectors with at least 2 different selectable markers makes the system more flexible as then, in most cases, at least one selectable marker that is present in the Acceptor vector will not be present in the Donor vector and could be chosen for selection after a subcloning reaction. Flexibility arises from the fact that more modes of operation to generate a Donor vector from multiple reactions between pre-existing Entry Vectors prior to nucleic acid molecule transfer into an Acceptor vector to generate a Destination vector become possible because these modes of operation also need to change the selectable marker from subcloning step to subcloning step between said Entry vectors and are not restricted anymore in a way that a defined selectable marker, the one of the Acceptor vector, has to be avoided from being used for creation of the Donor vector for said Acceptor vector. For distinguishing bacterial clones carrying an Acceptor vector from bacterial clones carrying the desired Destination vector, the nucleic acid fragment present in the Acceptor vector that should be replaced by the nucleic acid molecule from the Donor vector carries a reporter gene and is flanked by divergent type IIS restriction endonuclease recognition sites (cf., Entry vector 5 of FIG. 4B where NAM3 is flanked by divergent Esp3I recognition sites and therefore can be replaced by any nucleic acid fragment inserted in an Donor vector via compatible combinatorial sites). Such reporter gene may be the lacZα gene that encodes the alpha fragment of beta galactosidase including promoter (lacP/Zα), the gene for green fluorescent protein (GFP) or for yellow fluorescent protein (YFP), a suicide gene like ccdB, to name only a few illustrative examples.

An example, without limitation, for a suitable way to create a Destination vector by transfer of one nucleic acid molecule is (see also FIG. 2):

1. Mixing the Donor vector with an Acceptor vector in the presence of a type IIS restriction endonuclease and ligase and incubating in a buffer at a temperature where both enzymes are active. (The fact that the resulting Destination vector is precluded from the reaction because the reverse reaction is not possible due to the lack of the recognition sites of those type IIS restriction endonuclease(s) present in the reaction mixture is an advantage over the GATEWAY™ system where an equilibrium forms between the vectors introduced into the reaction and the desired vector reaction products because the reverse reaction is possible as well thereby leading to reduced Destination vector yield.)

Alternatively, the nucleic acid molecule can also be transferred from a Donor vector where it is placed between 2 convergent type IIS restriction endonuclease recognition sites that cleave at the combinatorial sites into an Acceptor vector which has (two respective) combinatorial sites that are cleaved by type IIS like restriction endonucleases. In such an embodiment, a Donor and an Acceptor vector are mixed and reacted with the corresponding type IIS restriction and the type IIS like restriction endonucleases, respectively, in the presence of ligase. For this purpose, the mixture containing the at least one Donor vector and at least one Acceptor vector and the 3 enzymes is incubated in a buffer at a temperature where all three enzymes are active.

2. Transforming bacteria, such as E. coli, with the reaction mixture and plating out on plates that contain preferably a substance for selection of the resistance gene present in the Acceptor/Destination vector and, if required, a further substance that allows to detect the reporter gene encoded by the Acceptor vector.

3. Isolating plasmid DNA from a clone that carries the Destination vector for further experiments.

When the nucleic acid molecule to be transferred carries an internal recognition site for the type IIS restriction endonuclease, the aforementioned step 1 may be modified so that, after restriction, type IIS restriction endonuclease is heat inactivated and ligase is subsequently added to the reaction. In general, however, internal restriction sites pose no problem as shown in Experimental Example 5, at least as long as the overhang that is produced is not identical to the overhangs produced at the combinatorial sites.

It should be emphasized here that this strategy is not only useful to create Destination vectors by the transfer of one target nucleic acid molecule only but also a plurality (i.e. at least two) of nucleic acid molecules may be transferred in one step by the strategy of the invention (cf., FIG. 3, FIG. 9C or FIG. 10). Using the products and methods of the invention, the generation of whole operating expression vectors (plasmids, viruses and the like) can be considered as a simple combinatorial problem, in which individual nucleic acid molecules only need to be combined via appropriate (predetermined) combinatorial sites.

In a first approach, it may be advantageous that the combinatorial sites used for construction of the Entry vector are either different from the combinatorial sites used for assembly of the nucleic acid molecules (other than shown in the Example of FIGS. 1 and 2), or at most partially overlapping with the sequence of the combinatorial sites in order to find a compromise between getting combinatorial site variability for assembly flexibility and keeping the sequence constraints from the combinatorial sites for the final assembly minimal. This strategy allows inserting the same nucleic acid molecule in parallel into different Entry vectors via the same combinatorial sites. Excision of the nucleic acid molecule from each Entry vector, however, equips said nucleic acid molecule with different cohesive ends, thereby allowing its positional allocation in a directed assembly with other nucleic acid molecules. The combinatorial site at the 3′ end of the first nucleic acid molecule has to be the same as the combinatorial site at the 5′ end of the second nucleic acid molecule (see FIG. 3). When more than 2 nucleic acid molecules have to be assembled, Entry vectors with further combinatorial sites are provided so that the combinatorial site at the 3′ end of the second nucleic acid molecule is the same as the combinatorial site at the 5′ end of the third nucleic acid molecule and so on. Exemplary applications for this parallel mode of operation include, but are not limited to, the generation of artificial polycistronic operons or the de novo synthesis of plasmid vectors from individual nucleic acid molecules (cf. FIG. 9).

The operating conditions of the cloning method/system usually eliminate type IIS recognition sites upon formation of the Destination vector. If, however, a first Entry vector contains a nucleic acid molecule together with two (2) divergently oriented type IIS recognition sites (=BsaI in Entry vector 1 of FIG. 4A) and such insertion is transferred into a second Entry vector carrying also two (2) divergently oriented type IIS recognition sites (=Esp3I in Entry vector 2 of FIG. 4A) with combinatorial sites compatible to combinatorial sites defined by the convergently oriented type IIS recognition sites in the first Entry vector (=Esp3I in Entry vector 1 in FIG. 4A), a third Entry vector is then generated that is able to take up a further nucleic acid molecule (FIG. 4A). By repeating this procedure with Entry vectors similar to the first Entry vector from above carrying further nucleic acid molecules, Entry vectors may be sequentially built up to assemble a plurality of nucleic acid molecules representing novel functional units. The outer (donor) combinatorial sites (defined e.g. by the type IIS restriction endonuclease SapI in FIG. 4) are retained throughout the sequential assembly procedure while the inner (acceptor) combinatorial sites are from integration step to integration step alternately defined by two different divergently oriented type IIS restriction endonuclease recognition sites (Esp3I and BsaI in FIG. 4). The integration of the last nucleic acid molecule will not carry along 2 divergently oriented type IIS restriction endonuclease recognition sites thereby leading to the formation of a Donor vector instead of a further Entry vector and the outer combinatorial sites may now be used for insertion of the finally assembled unit into a designated Acceptor vector thereby creating a Destination vector. A typical application for this sequential mode of operation is the combinatorial synthesis of vectors, in which multiple nucleic acid molecules such as but not limited to affinity tags, secretion signals and fusion partners are assembled.

If the nucleic acid molecule to be transferred into an Entry vector is arranged in between the divergently oriented type IIS recognition sites (cf. nucleic acid molecule 3 between Esp3I in FIG. 4B), the further Entry vector to be generated is for uptake of an nucleic acid molecule that substitutes said nucleic acid molecule (FIG. 4B).

A further advantageous application of the methods of the invention is the ability for simple site-directed mutagenesis (substitutions, deletions and additions of nucleic acid sequences as well as simultaneous combinations thereof) of nucleic acid molecules during e.g. the generation of a Donor vector (see FIG. 5). Such an application is e.g. useful for eliminating “internal” recognition sites for the operating type IIS restriction endonucleases from nucleic acid molecules (e.g. target genes) that otherwise may hinder to exploit efficiently the subsequent methods of the invention for modular assembly of nucleic acid molecules. Such site directed mutagenesis can, for example, also be used for optimization of codon usage or the facile generation of deletions, additions, fusion proteins and chimeras. The mutagenesis method of the present invention does not rely—in contrast to conventional PCR mutagenesis—on the necessary presence of gene internal restriction sites but takes advantage of the fact that the sequences of the cohesive ends necessary for directed ligation of the two PCR products can be freely chosen and the type IIS restriction endonuclease recognition sites for creation of said cohesive ends may be positioned so that they are eliminated from the final product. Thus, the mutagenesis method of the present invention provides a convenient means for directed mutagenesis at any desired chosen site of any given target nucleic acid.

### Manufacture of Entry and Acceptor Vectors and Provision of the Necessary Overhangs for Uptake of a Nucleic Acid Molecule

In one embodiment, an Entry and/or Acceptor vector is provided in either circular or linear form and possesses divergent type IIS restriction endonuclease recognition sites on behalf of which the overhangs (cohesive ends) at the combinatorial site can be generated after cleavage with the corresponding restriction endonuclease for uptake and insertion of a nucleic acid molecule excised from a Donor vector.

In a further embodiment, Entry and Acceptor vectors are provided in either circular or linear form and possess type IIS like restriction endonuclease recognition sites on behalf of which compatible overhangs can be generated after cleavage with the corresponding type IIS like restriction endonuclease(s) for uptake and insertion of a nucleic acid molecule excised from a Donor vector at the combinatorial site(s).

In yet a further embodiment, Entry and/or Acceptor vector are provided in linear form and possess overhangs for uptake and insertion of a nucleic acid molecule excised from a Donor vector at the combinatorial site(s). In these embodiments, the respective linear Entry or Acceptor vector does not contain a recognition site for a type IIS restriction endonuclease.

In another embodiment, Entry and Acceptor vectors are provided in linear form and possess overhangs for uptake and insertion of a nucleic acid molecule excised from a Donor vector at the combinatorial site(s), wherein said overhangs have been generated by one or more type IIS restriction endonucleases.

In still a further embodiment, Entry and Acceptor vectors are provided in linear form and possess overhangs for uptake and insertion of a nucleic acid molecule excised from a Donor vector at the combinatorial site(s), wherein said overhangs have been generated by one or more type IIS like restriction endonucleases.

In still a further embodiment, Entry and Acceptor vectors are provided in linear form and possess overhangs for uptake and insertion of a nucleic acid molecule excised from a Donor vector at the combinatorial site(s) whereby said overhangs have been generated by ligating a linker to the opened Entry or Acceptor vector. Said linker may be generated, without limitation, by annealing single stranded oligonucleotides or by excising a double stranded nucleic acid stretch from DNA with appropriate enzymes.

### Formation of Combinatorial Sites

The combinatorial sites of the respective nucleic acid molecule (which can be the molecule of interest or a vector used in the present invention) can typically be formed as an overhang selected from the group consisting of a nucleotide sequence of 5 bases in length, a non-palindromic nucleotide sequence with 4 bases in length, a nucleotide sequence of 3 bases in length, a non-palindromic nucleotide sequence of 2 bases in length, and a nucleotide sequence of 1 base in length.

The nucleotide sequence of the overhang can have any suitable sequence, for example, GAATG, AAATG, AAAGG, GGGGA, GGGGC, GGGTC, GGGCA, TAAGC, TGCTC, CCCTC, GAGAG, ATCGG, AAGGG, GCCCT, GCCGC, ATTGA, GAAAA, CCCGC, CTCCT, AATG, GGGA, TAAG, GAAT, AAAT, AAAG, GGGG, GGGT, GGGC, TGCT, GAGA, ATCG, GCTG, GGCT, TCCT, CCCT, CGCG, TGCT, TTTT, TCTC, TCCG, CCGC, CAAA, CTCC, ATTG, GAAA, ATG, GGG, AAT, TCC, TCT, AGC, TGC, CCC, GCT, TGG, GAA, GAG, AGG, AAA, ATA, CTT, CTC, TTG, GTT, TTT, ACT, TAO, CAA, CAT, GAT, CGT, CGC, TAA, TAG, TGA, TA, TG, GG, CC, CT, GA, AG, A, G, T, C and the respective complementary sequence.

### Kits

In accordance with the above disclosure the invention also provides a nucleic acid cloning kit. Such a kit can contain only at least one Acceptor vector or at least one Entry vector as described herein. It is also possible that the kit comprises in two separate parts at least one Acceptor vector and at least one Entry vector. Further, such a kit can contain also at least one Entry vector for upstream fusion and one Entry vector for downstream fusion.

An (replicable) Entry vector (that can be offered in a kit alone and/or in combination with at least one Acceptor vector) in into which the at least one nucleic acid molecule of interest is to be inserted can carry two recognition sites for a at least one first type IIS restriction endonuclease and/or one at least one type IIS like restriction endonuclease. The at least one nucleic acid molecule of interest can be excised from the at least one Entry vector at two combinatorial sites with one (same) or more (different) cohesive ends that are formed by the at least one first type IIS and/or type IIS like restriction endonuclease.

An at least one Acceptor vector (that can be offered in a kit alone and/or in combination with at least one Entry vector) comprises at least one recognition site for a second type IIS restriction endonuclease and/or type IIS like restriction endonuclease. In addition the Acceptor vector provides combinatorial sites identical to the two combinatorial sites present in an Entry vector from which an inserted at least one nucleic acid molecule of interest can be transferred (i.e., a Donor vector generated from the Entry vector).

A nucleic acid cloning kit of the invention can comprise a plurality of Acceptor vectors with identical combinatorial sites, for example, in order to provide a plurality of different genetic surroundings for a target nucleic acid to be expressed (cf. also FIG. 11 in this regard).

The Entry vector can be provided in a kit either in circularized or linearized form. When provided in linearized form, the Entry vector may have been opened/linearized in any suitable way as long as the linearized Entry vector is capable of ultimately providing the desired (free) cohesive ends. As described above, the Entry vector may have been opened for example, but not limited thereto by cleavage of an restriction endonuclease, for example any regular type IIP restriction endonuclease at an arbitrary position between two of the at least one third (divergent) type IIS restriction endonuclease recognition sites. Thus, in this approach the desired/necessary cohesive ends for uptake of the nucleic acid molecule from the Donor vector or PCR product will be created by the at least one third type IIS or type IIS like restriction endonuclease during the reaction with the Donor vector or PCR fragment. Alternatively, in another embodiment of the kit, the linearized Entry vector may have been opened by cleavage of the at least one third type IIS restriction endonuclease so that the cohesive ends of the Entry vector comprise the combinatorial sites and are ready prior to the reaction with a Donor vector or PCR fragment for uptake of the nucleic acid molecule from the Donor vector or PCR fragment after cleavage with the at least one first type IIS restriction endonuclease.

In line with the above, also the Acceptor vector can be provided in a kit either in circularized form or in linearized form. When provided in linearized form, the Acceptor vector may have been opened/linerarized in any suitable way as long as the linearized Acceptor vector is capable of ultimately providing the desired (free) cohesive ends. As described above, the Acceptor vector may have been opened for example, but not limited thereto, by cleavage of an restriction endonuclease, any regular type IIP restriction endonuclease, at an arbitrary position between the two at least one second (divergent) type IIS restriction endonuclease recognition sites so that the necessary cohesive ends for uptake of the nucleic acid molecule from the Donor vector will be created by the at least one second type IIS or type IIS like restriction endonuclease during the reaction with the Donor vector. Alternatively, in another embodiment of the kit, the linearized Acceptor vector may have been opened by cleavage of the at least one second type IIS restriction endonuclease so that the cohesive ends of the Acceptor vector comprise the combinatorial sites and are ready prior to the reaction with the Donor vector for uptake of the nucleic acid molecule from the Donor vector after excision with the at least one first type IIS restriction endonuclease.

A kit of the invention can further comprise the one or more type IIS restriction endonucleases the recognition site of recognition sites of which the Entry or Acceptor vectors carries. In addition, the kit can also comprise buffer solutions that provide for suitable reaction conditions for the restriction endonuclease(s).

### FIGURES AND EXAMPLES

The embodiments of the invention are further illustrated by the following figures and non-limiting examples.

FIG. 1

FIG. 1 illustrates an example of a method to create a Donor vector by inserting a nucleic acid molecule of interest (=DNA molecule) into an Entry vector.

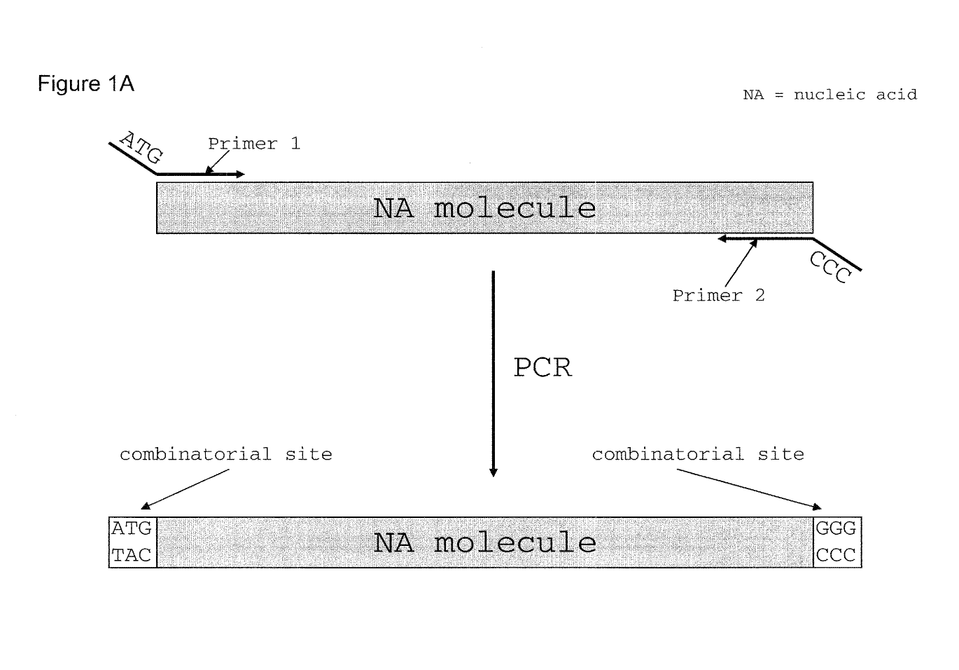

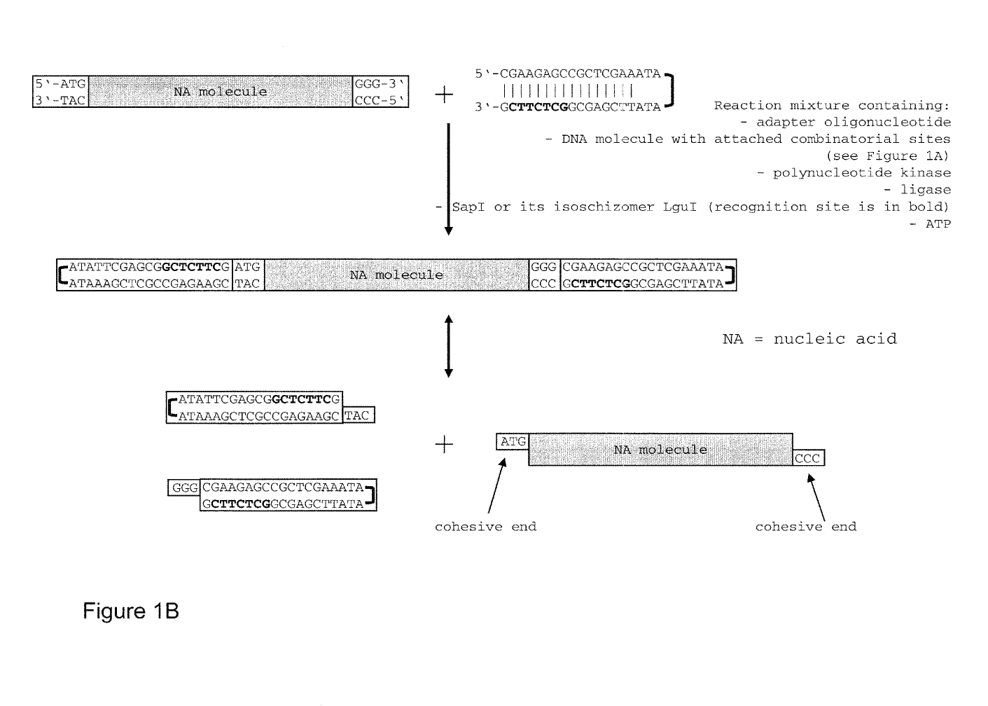

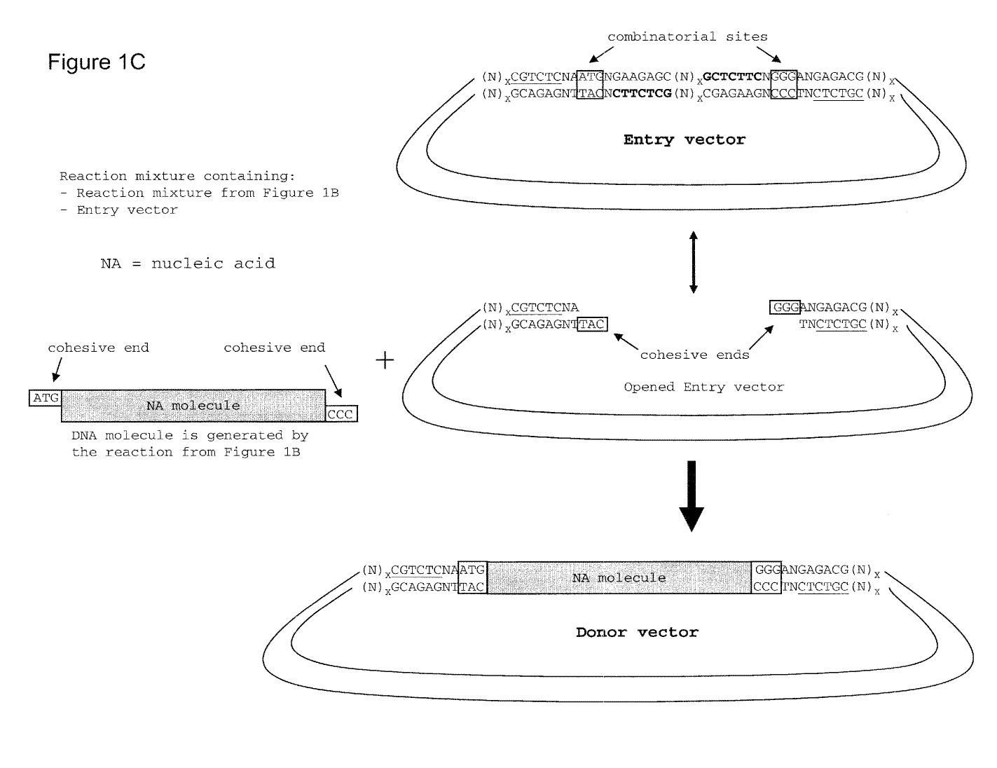

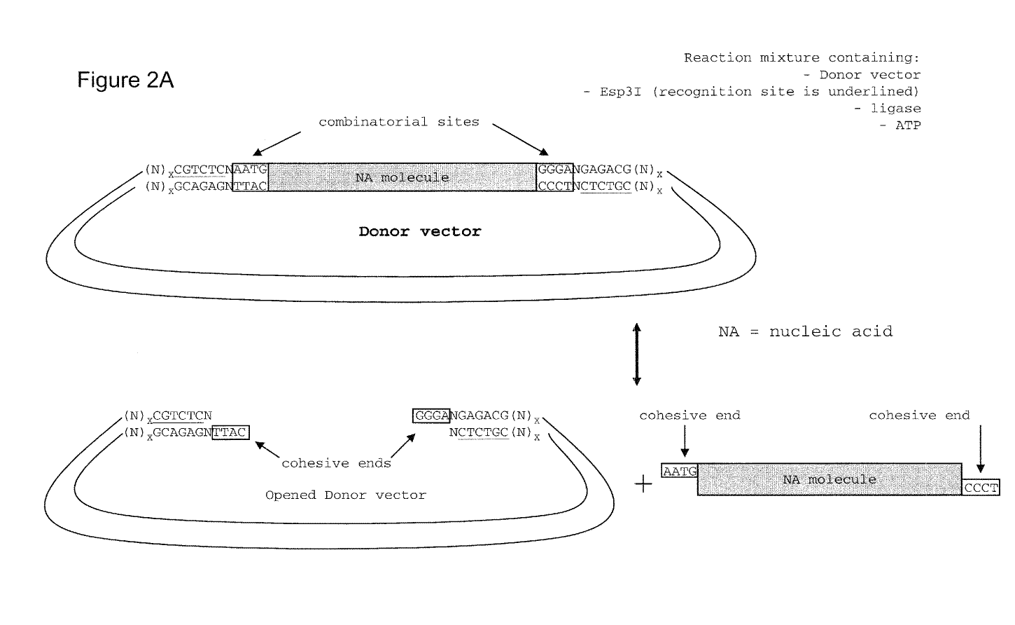

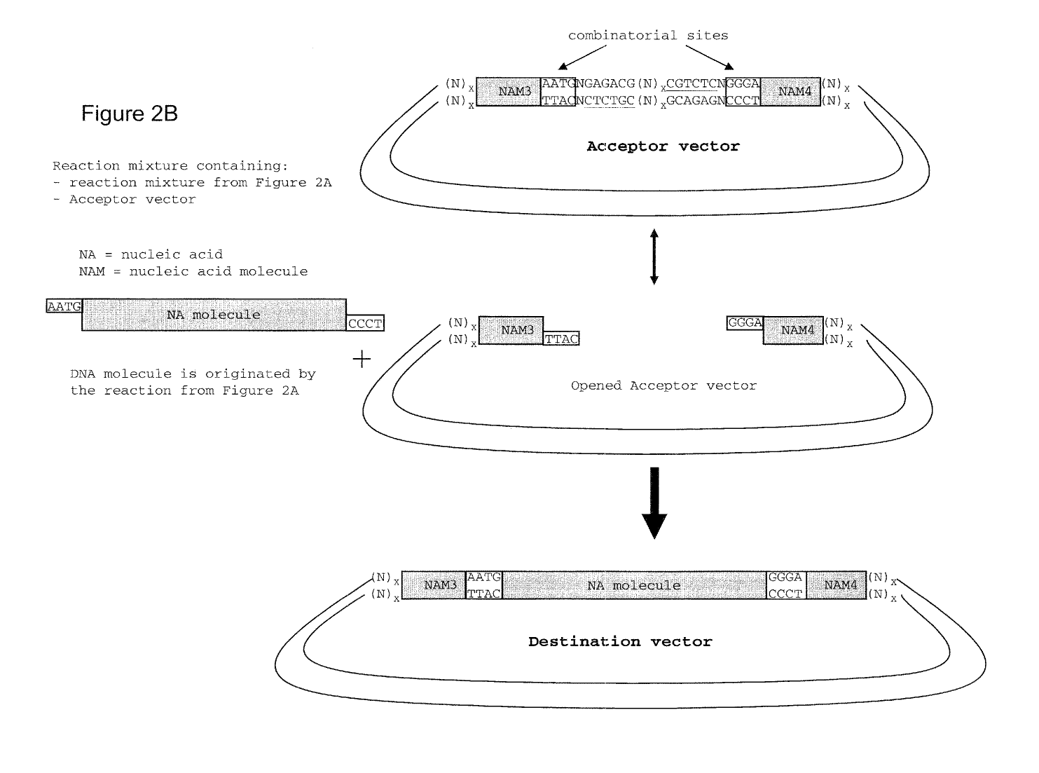

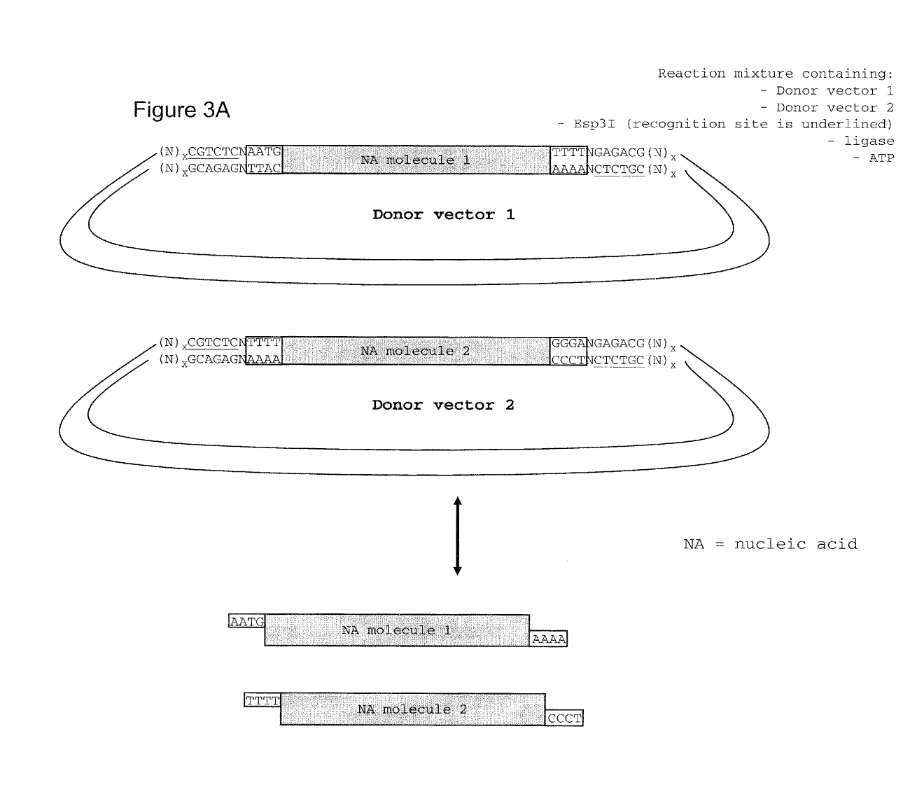

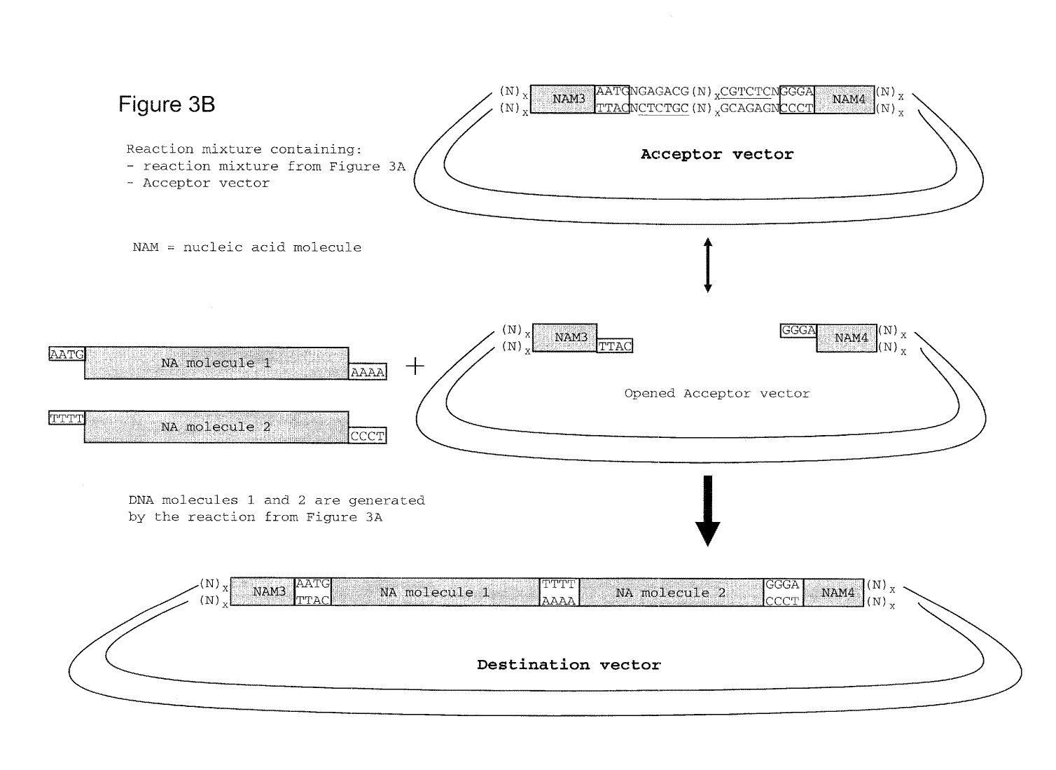

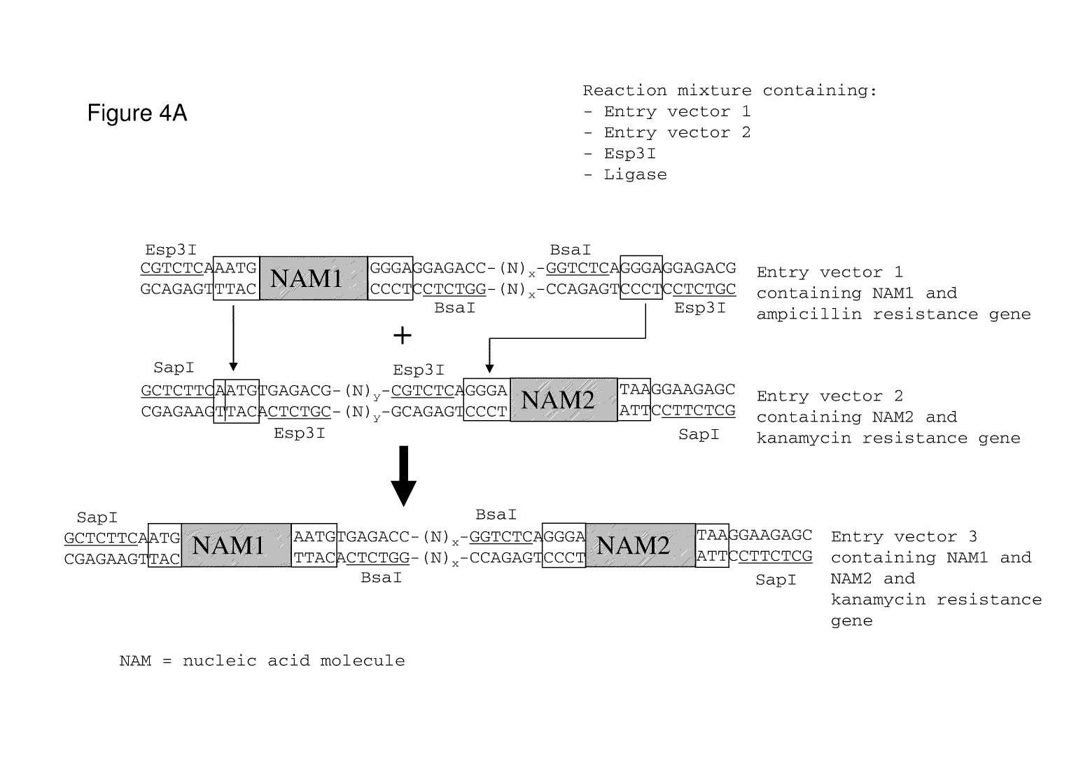

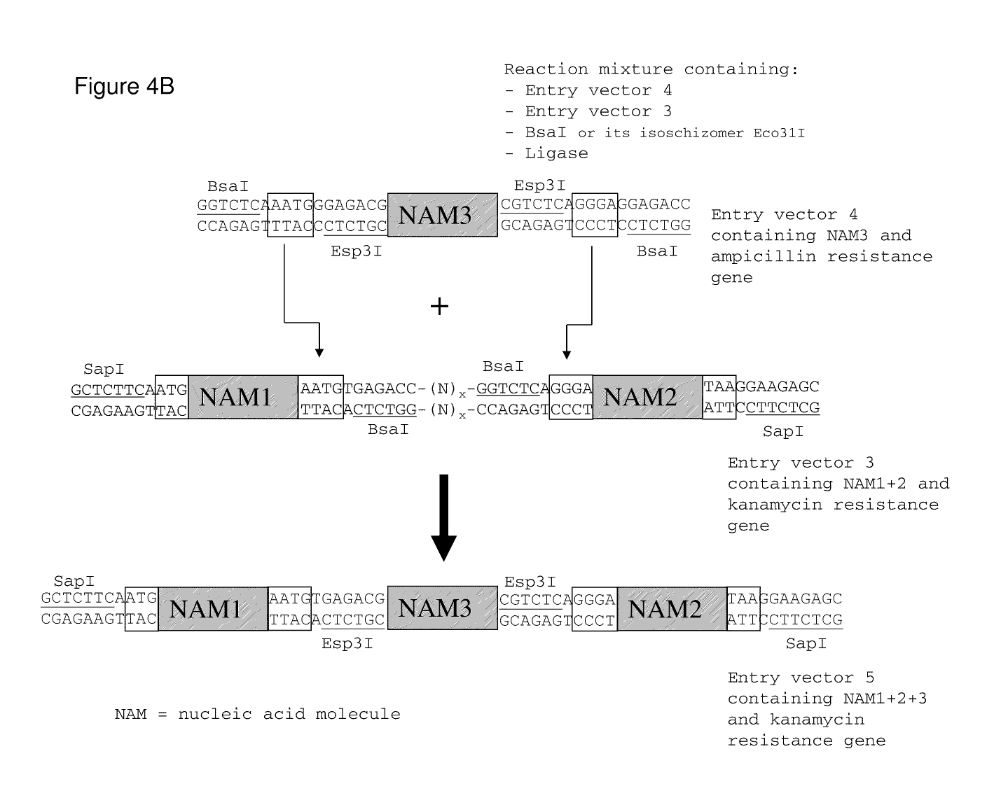

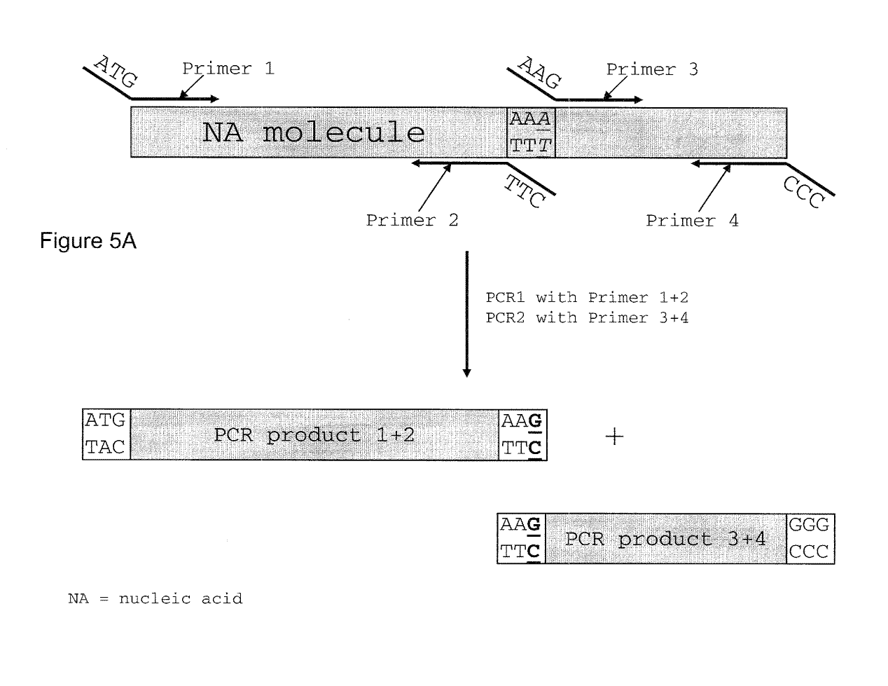

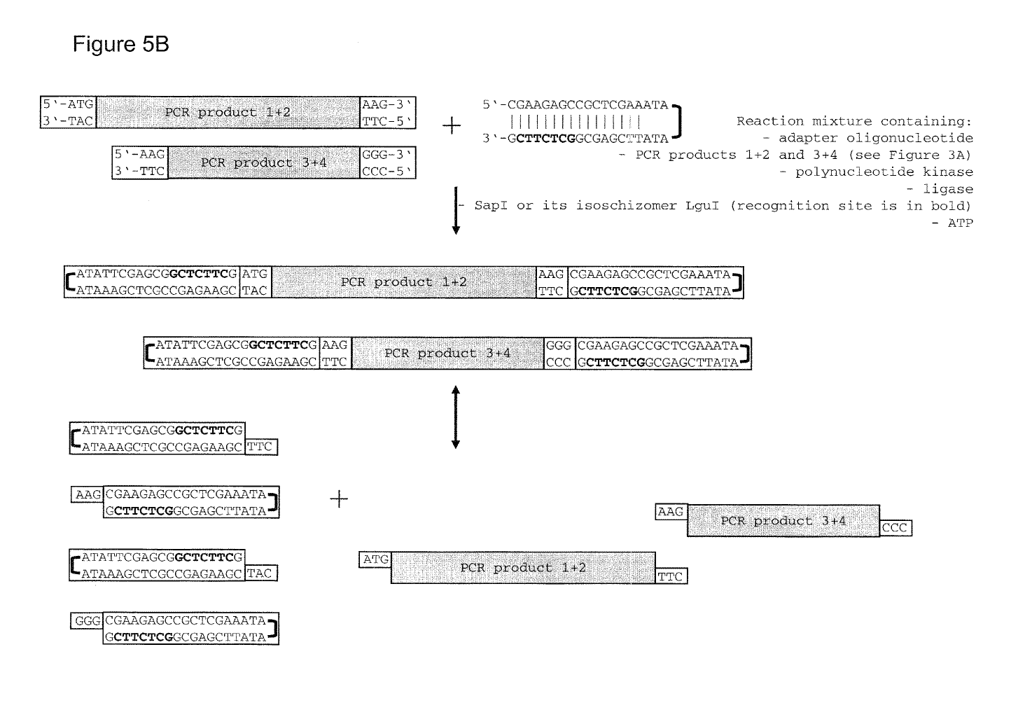

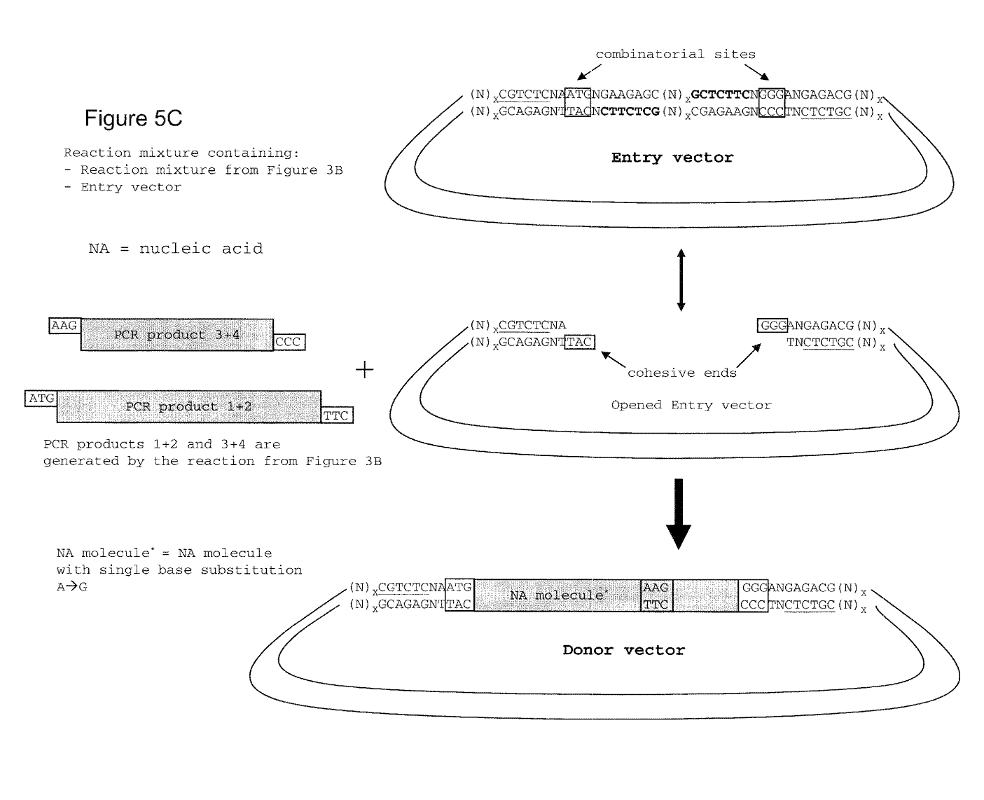

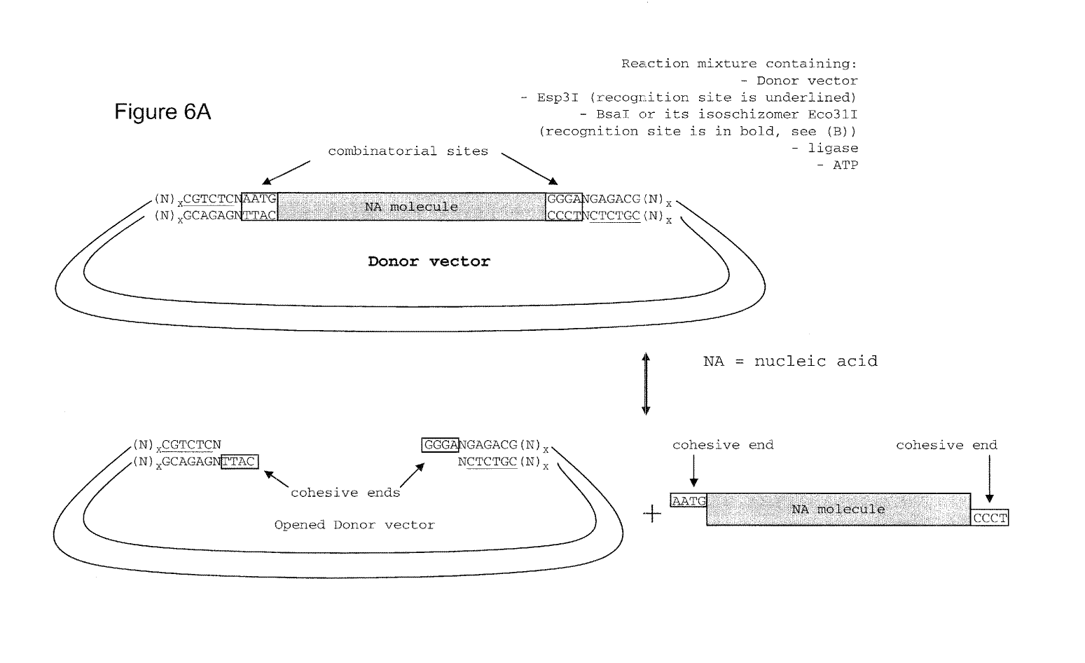

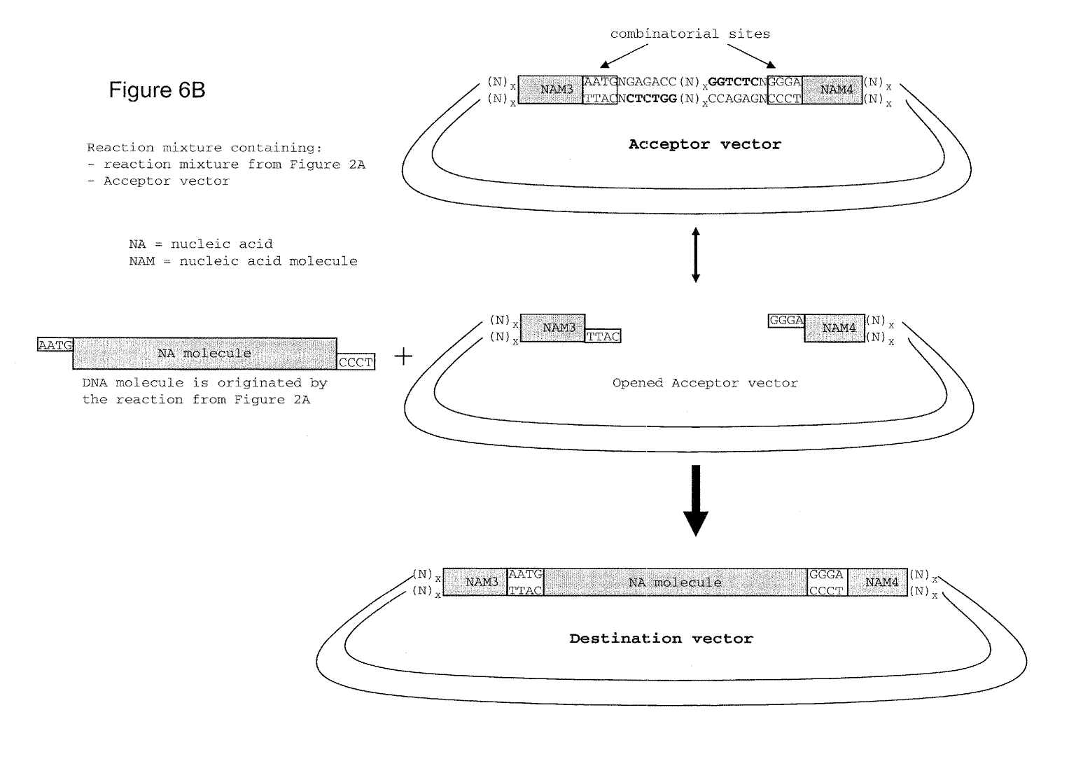

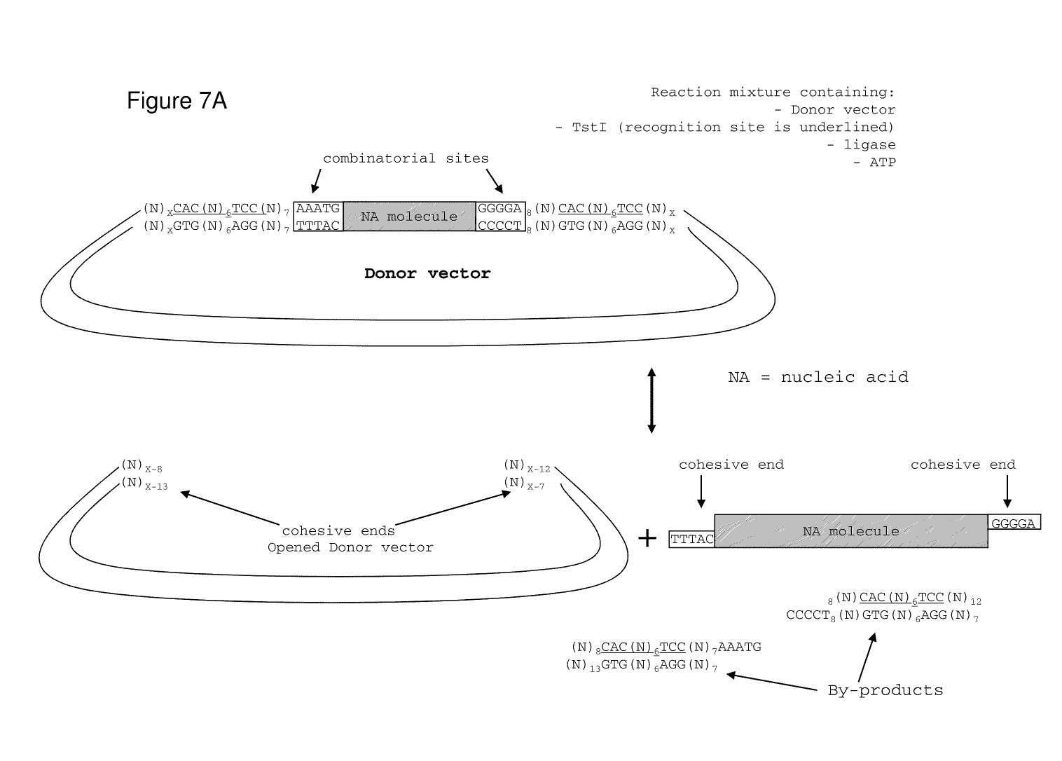

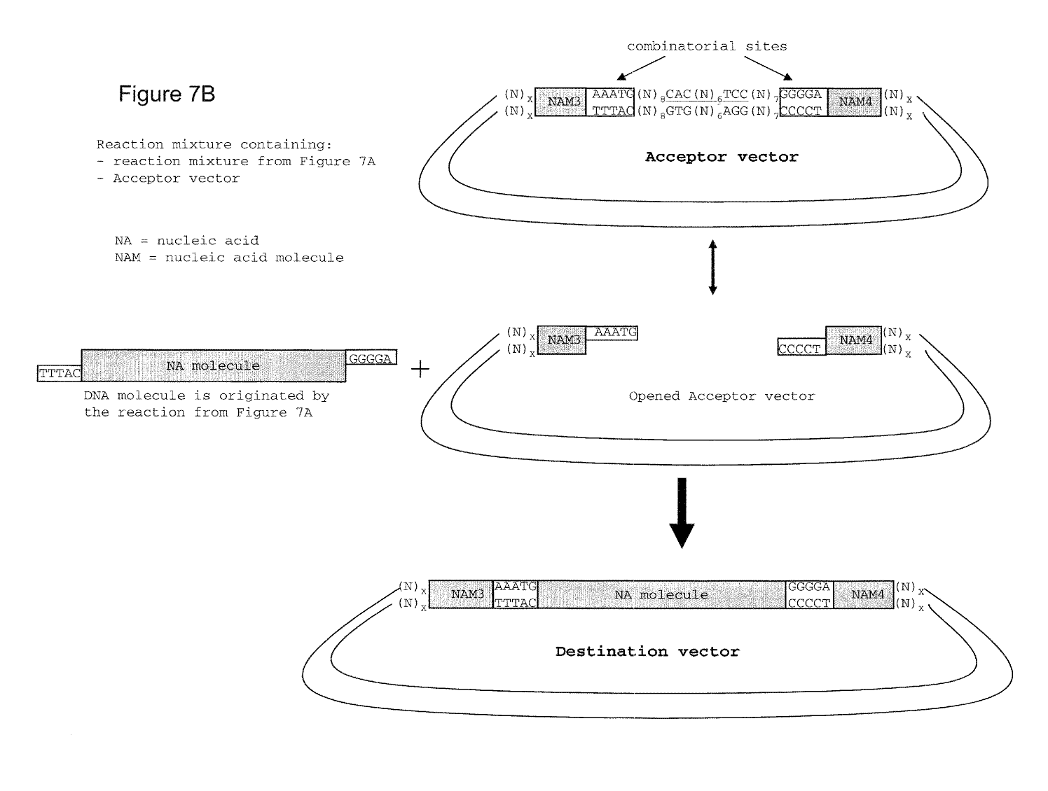

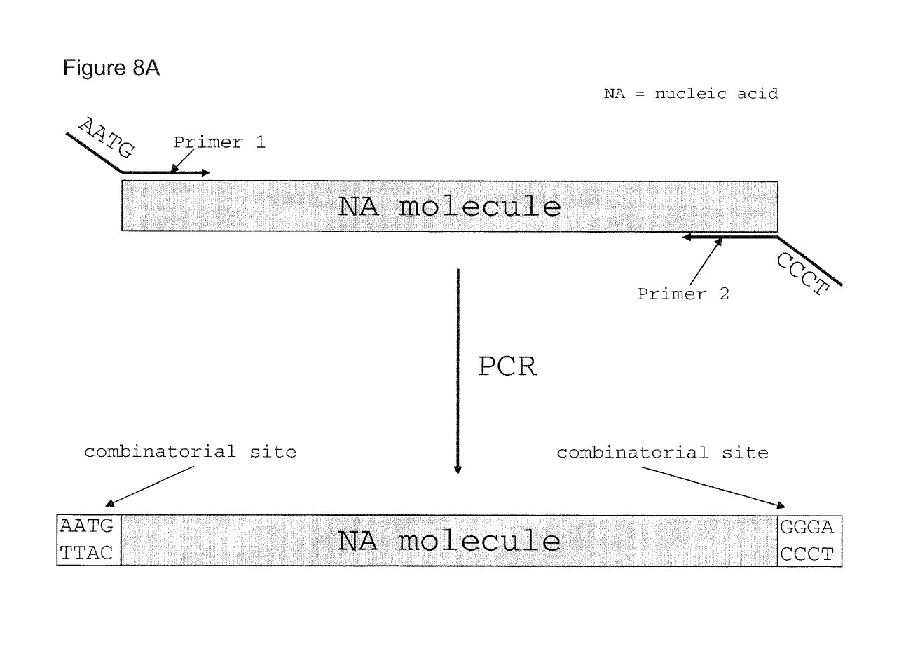

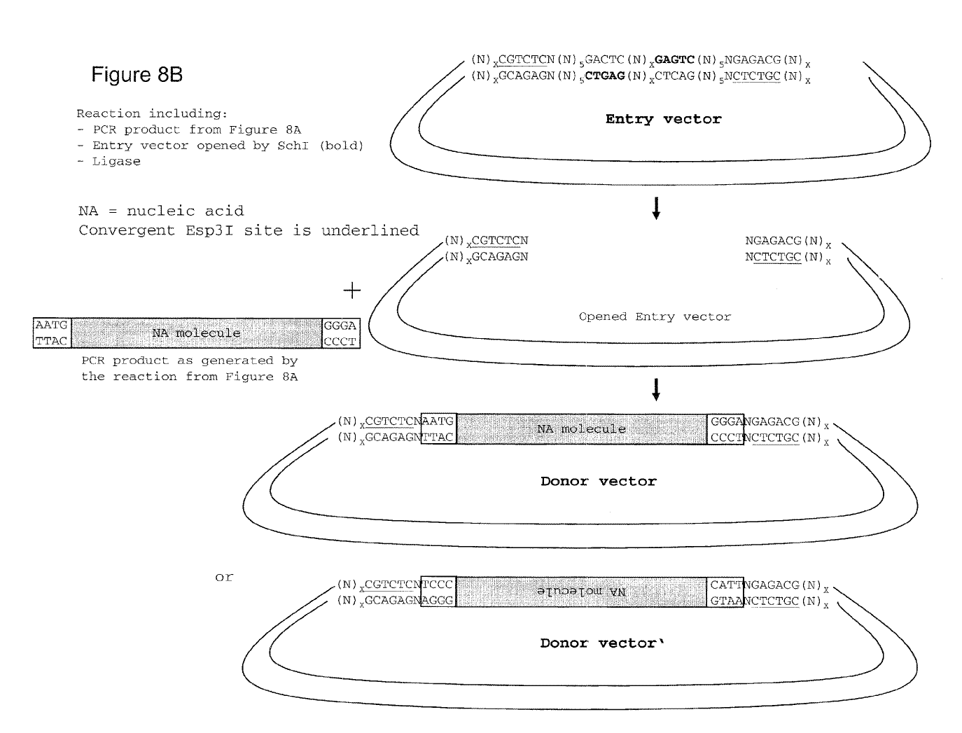

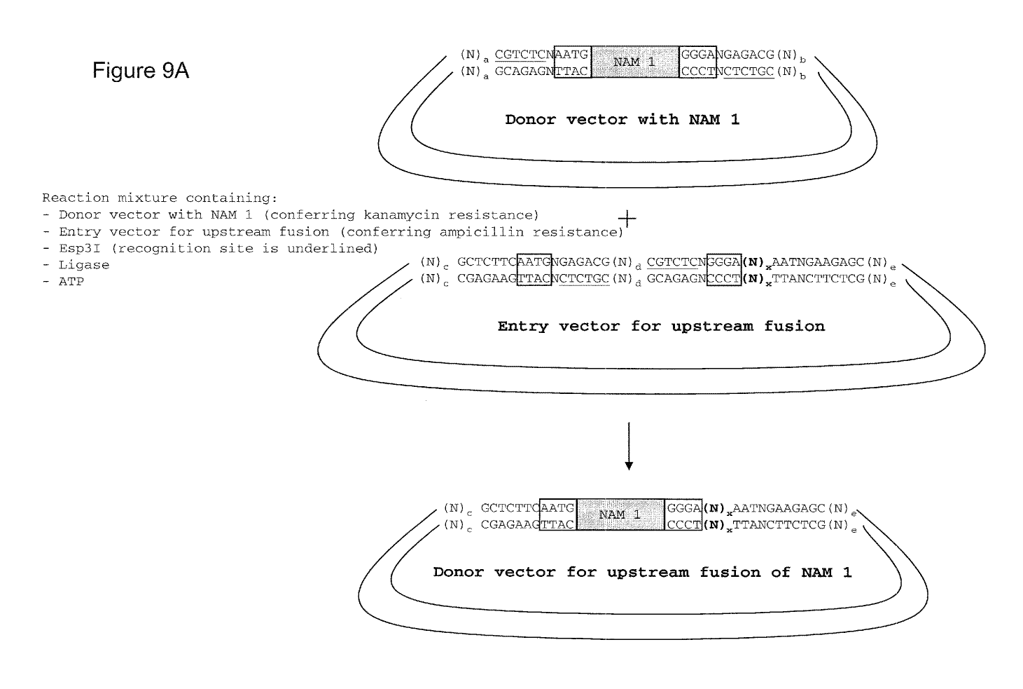

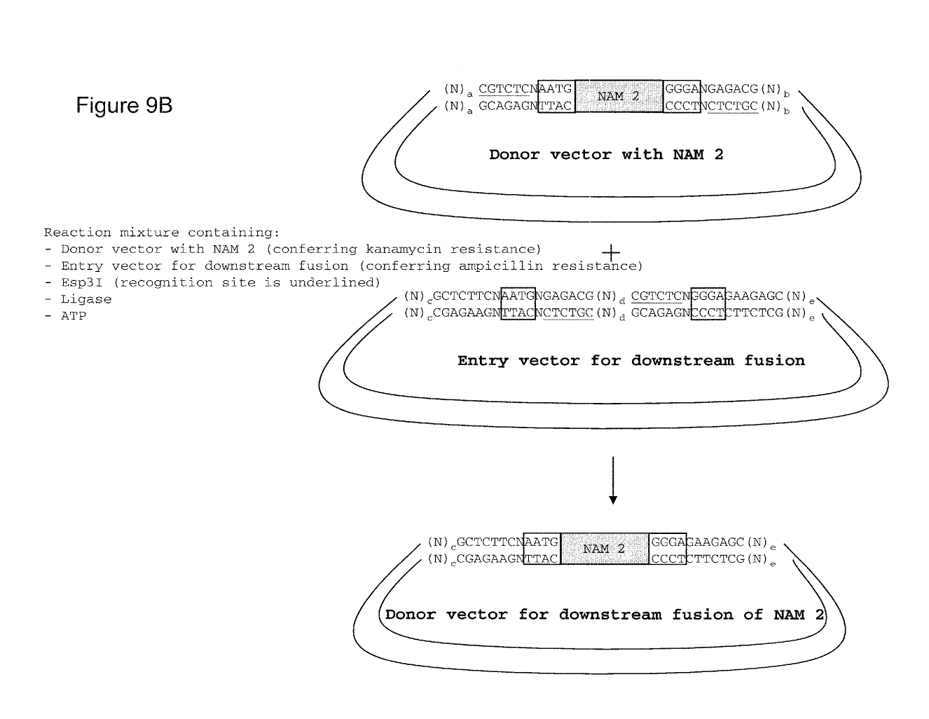

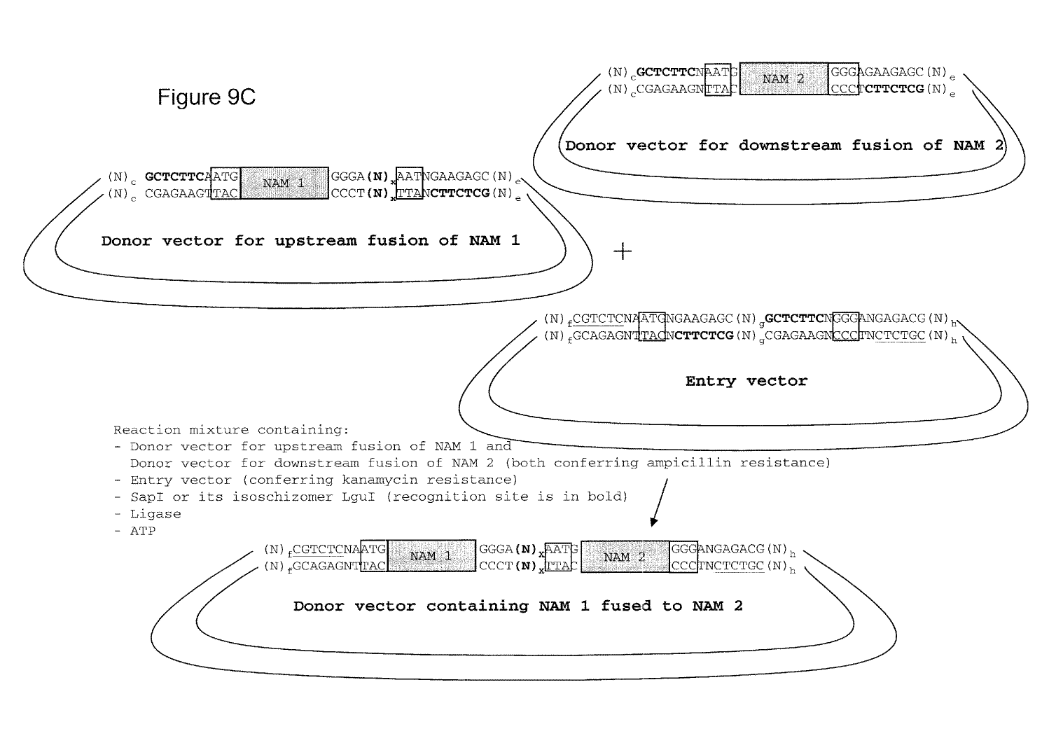

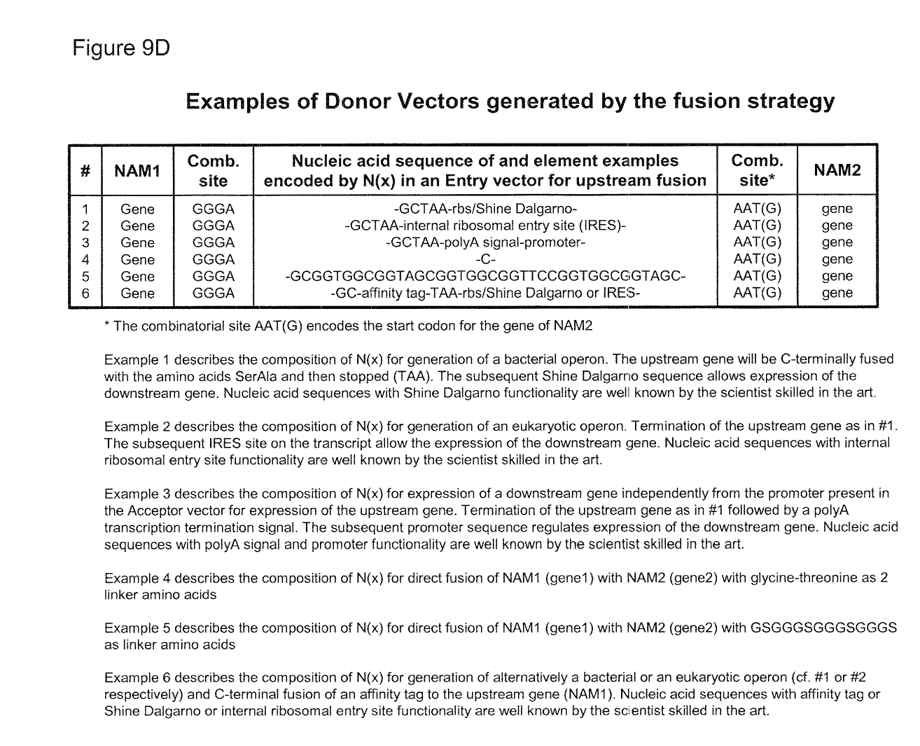

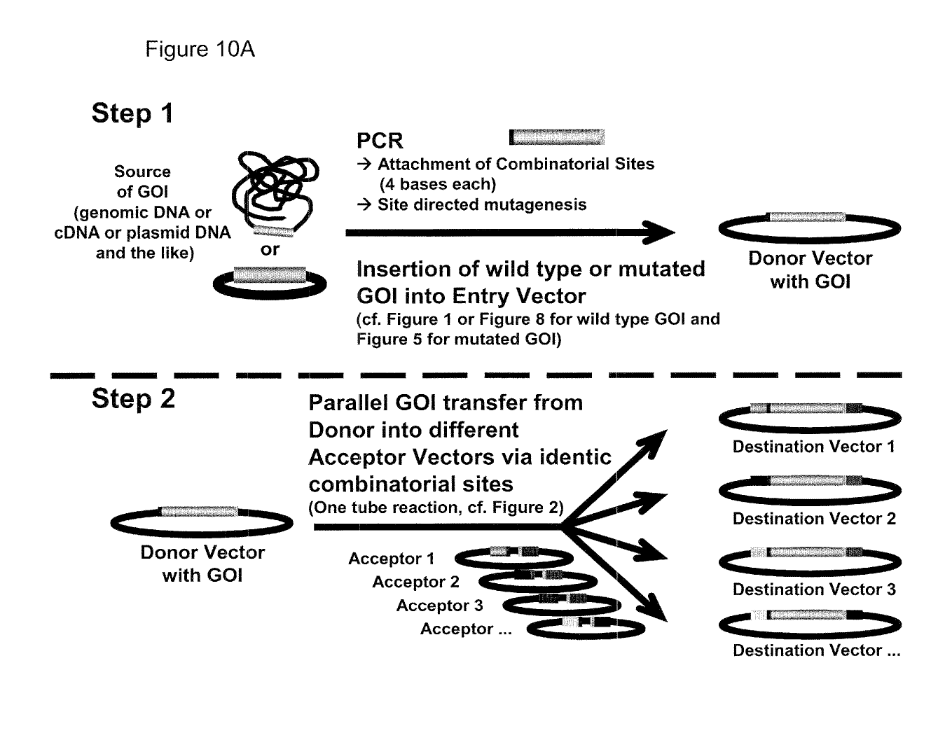

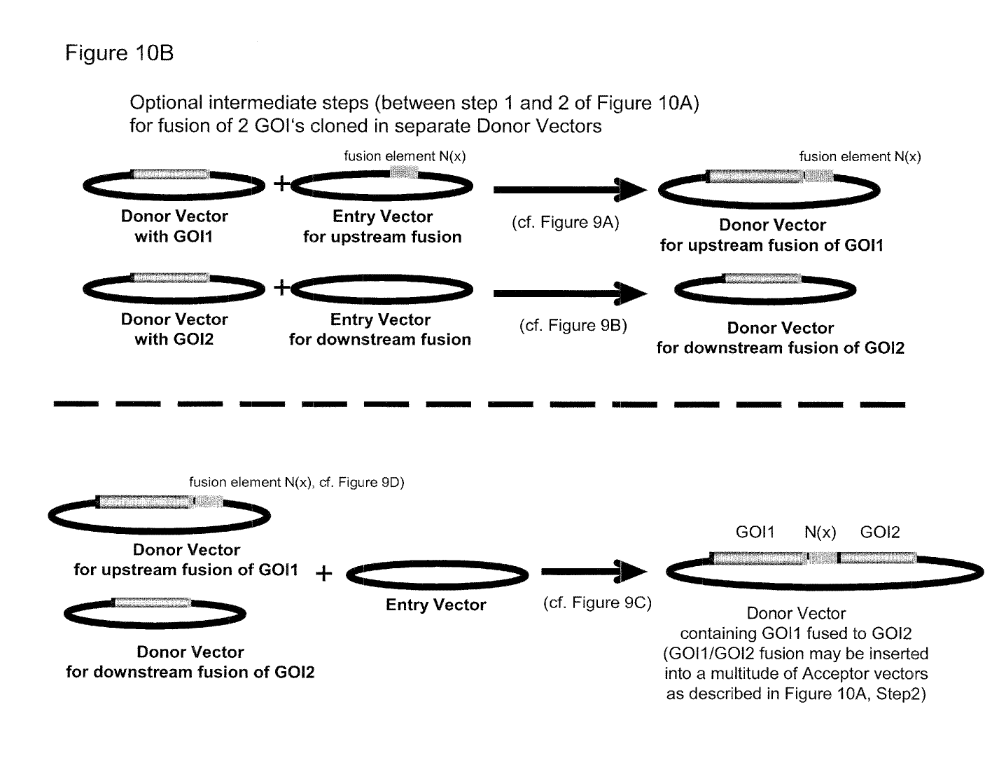

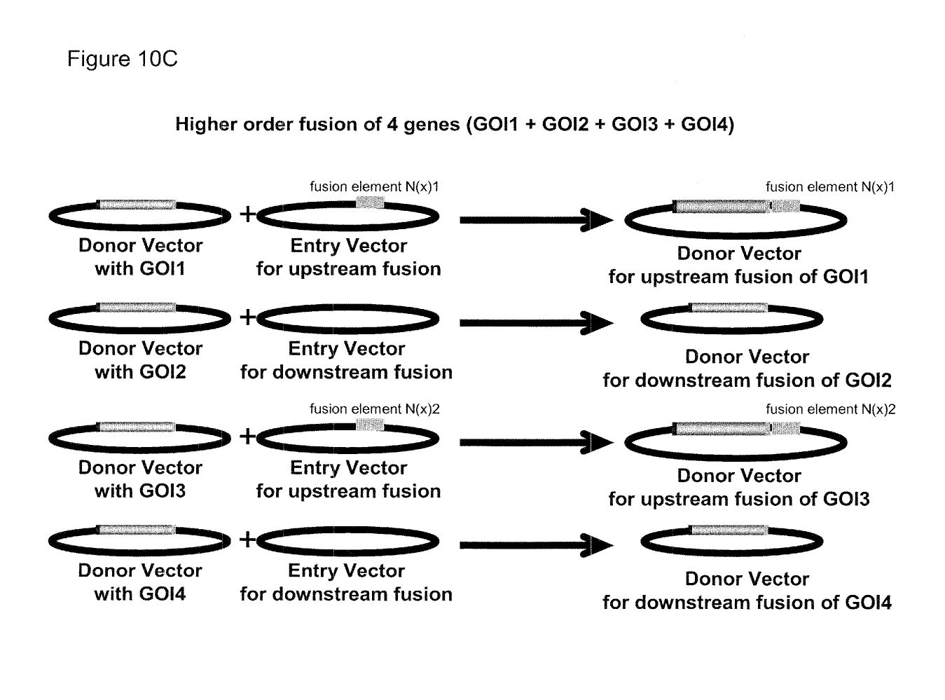

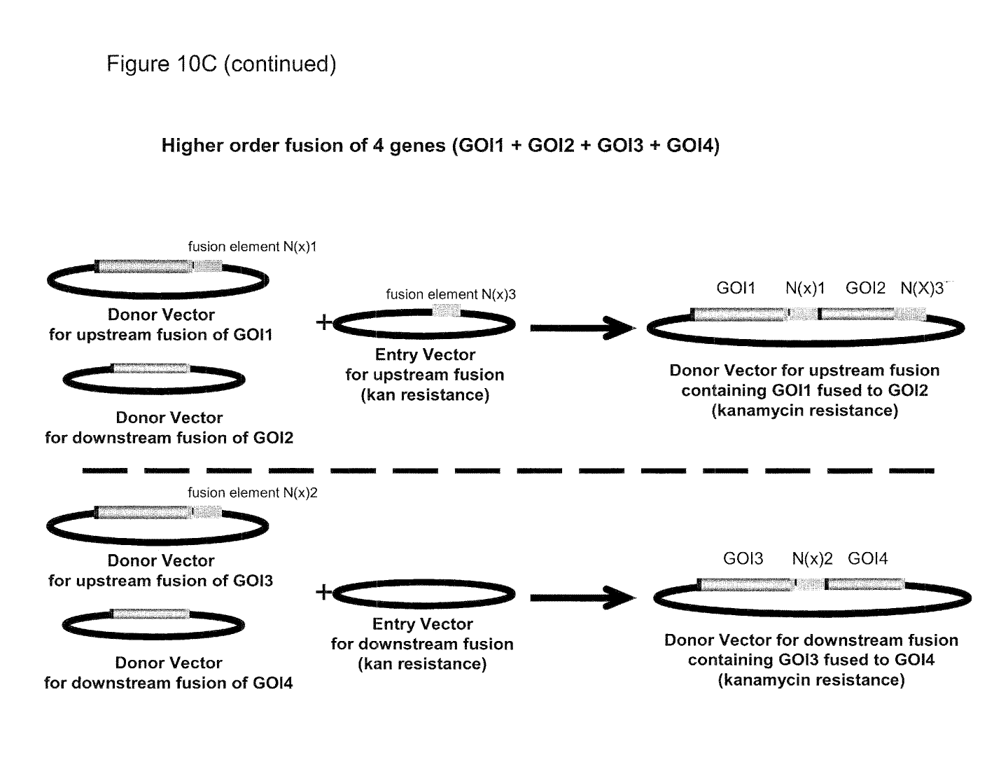

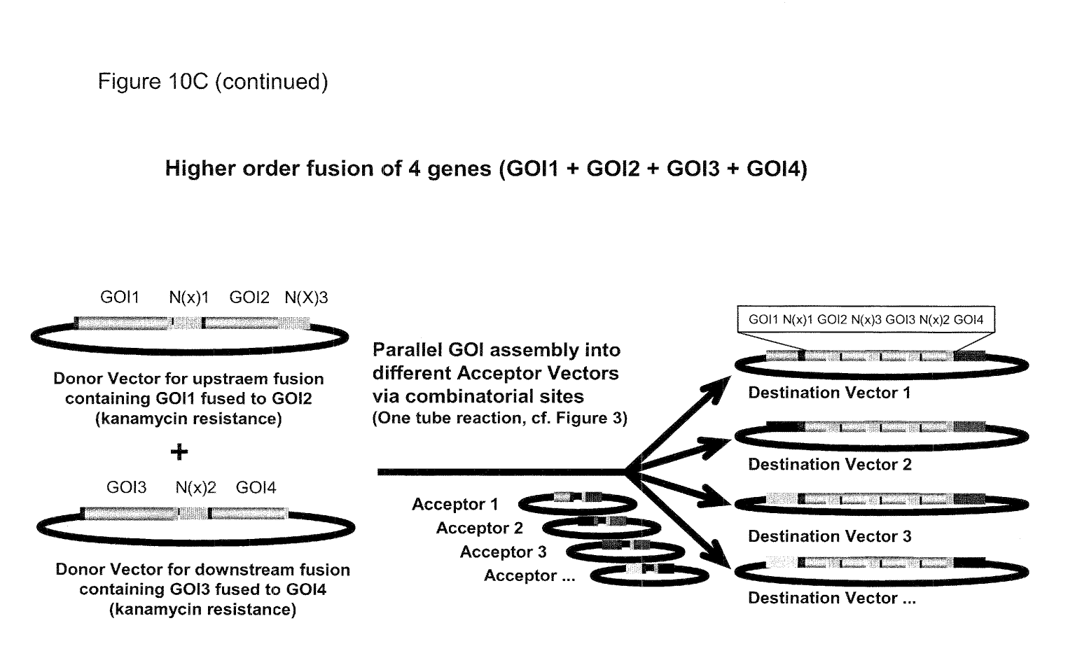

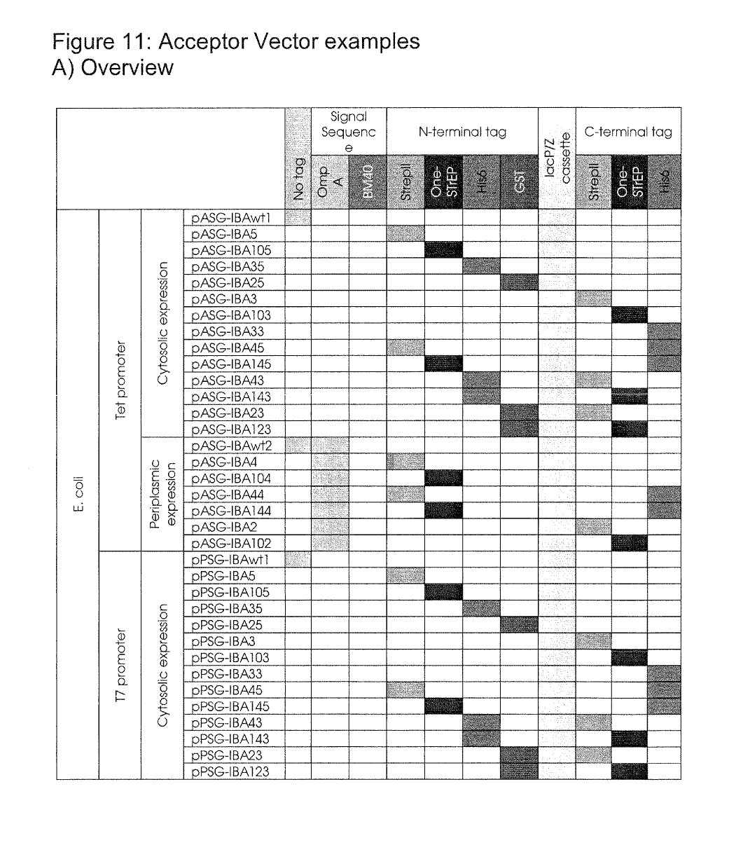

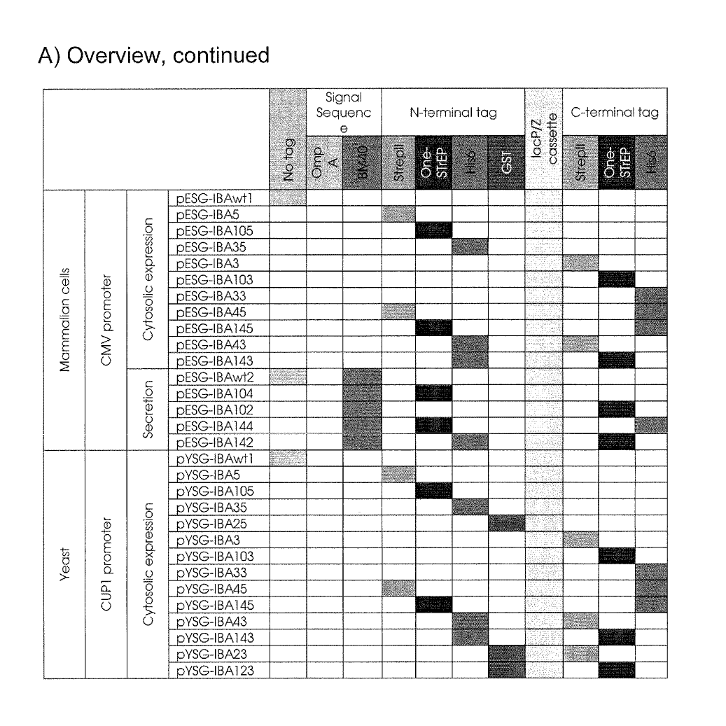

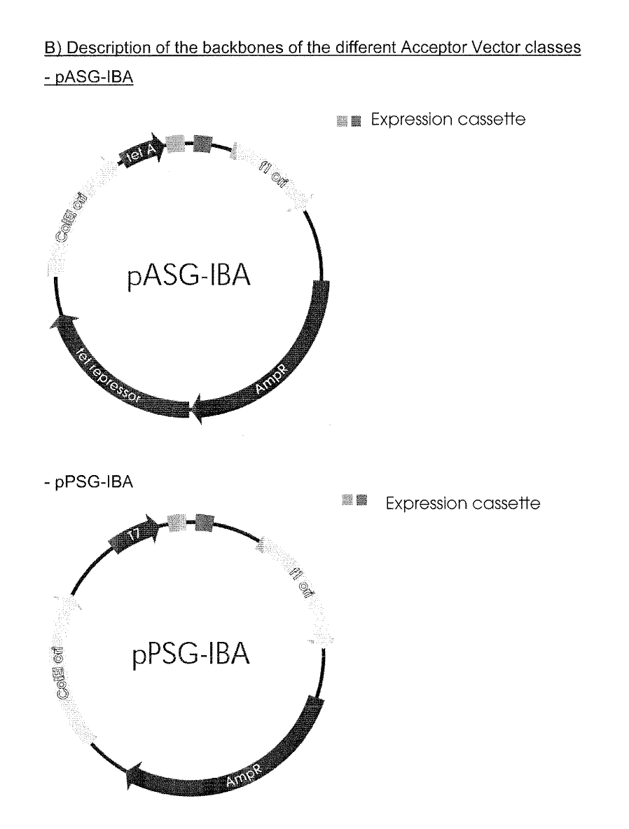

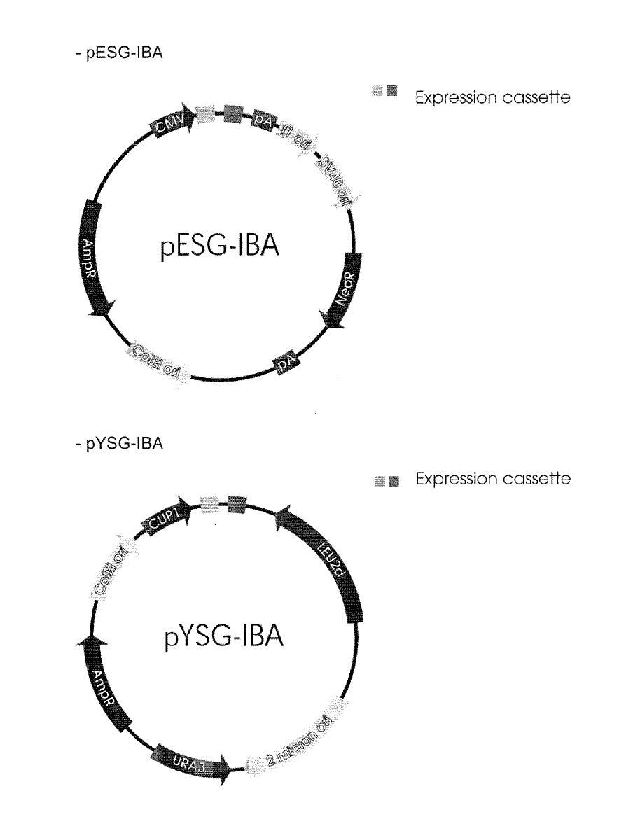

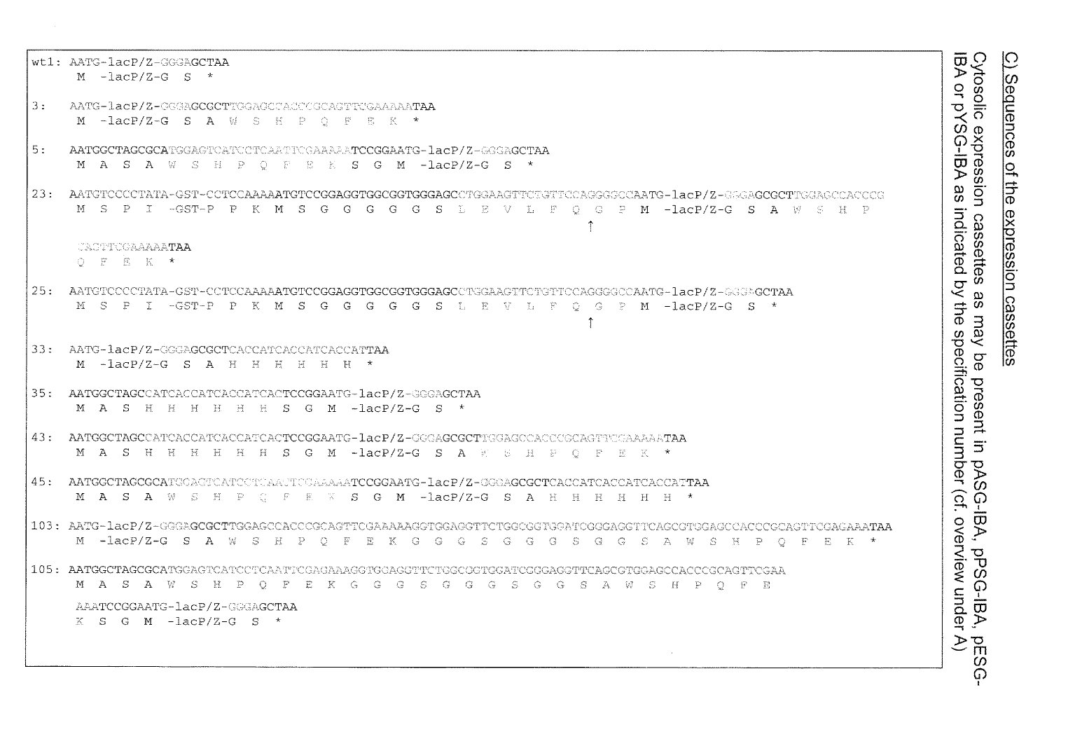

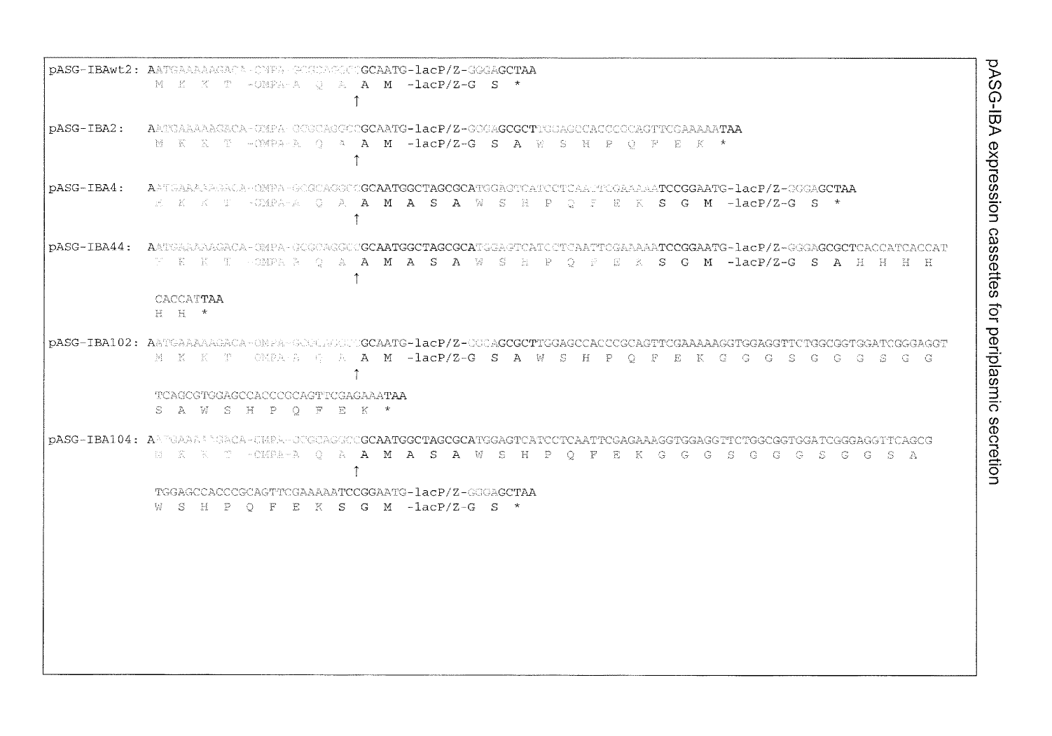

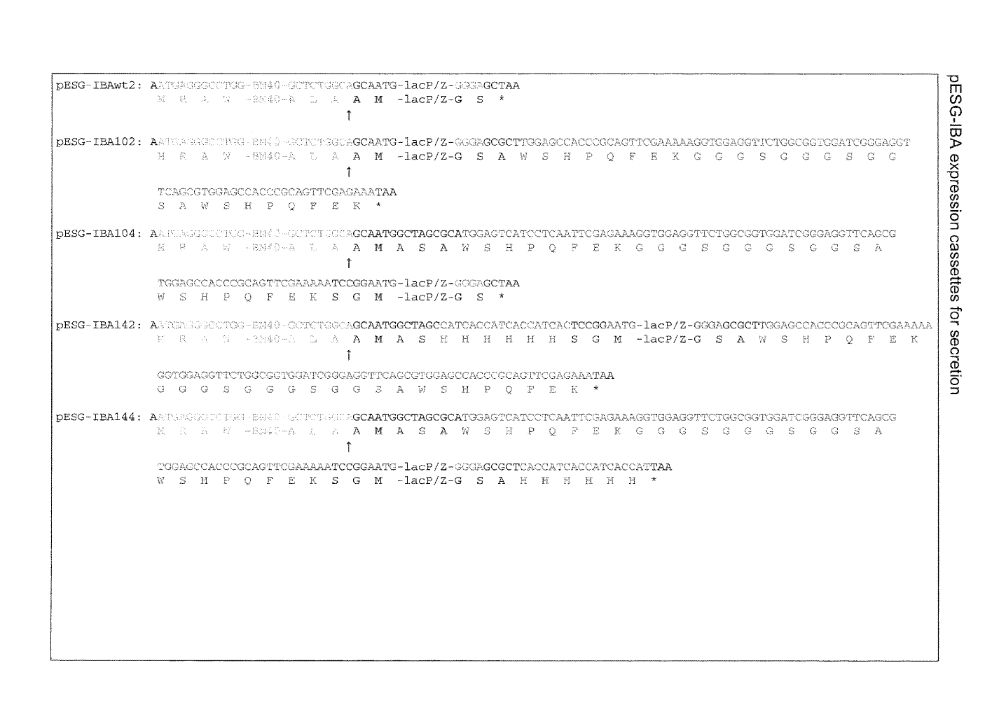

In a first step (FIG. 1A), the nucleic acid molecule of interest is modified at both ends to attach specific (predetermined) combinatorial sites. In this illustrative example, the whole combinatorial site is attached at this step by PCR using appropriate primers (Primer 1 and 2). Alternatively, only a part of the combinatorial site may be attached at this step and the other part may be provided by the adapter oligonucleotide described in FIG. 1B.

After PCR, the PCR products are purified and transferred into a reaction mixture (FIGS. 1B and 1C). Said reaction mixture contains (i) an adapter oligonucleotide (e.g. 5′-CGAAGAGCCGCTCGAAATAATATTCGAGCGGCTCTTCG) which provides the recognition site for a type IIS restriction endonuclease (e.g. SapI or LguI as shown in FIG. 1B) and, if wanted, also a part of the combinatorial site (=not the case in the actual example), (ii) a type IIS restriction endonuclease (e.g. SapI or LguI), (iii) DNA ligase (e.g. T4 DNA ligase), (iv) ATP, (v) a Donor vector with appropriate combinatorial sites and (vi) optionally polynucleotide kinase (e.g. T4 polynucleotide kinase) when synthetic oligodesoxynucleotides without 5′ phosphate are used. For the sake of clarity it is noted here that the recognition site of SapI is

| | | |
|---|---|---|
| 5′-GCTCTTC(N₁)↓ | and/or | ↓(N₄)GAAGAGC-3′ |
| 3′-CGAGAAG(N₄)↓ | | ↓(N₁)CTTCTCG-5′ |

meaning the cleavage site is located after the first nucleotide downstream the 3′-end of the recognition site 5′-GCTCTTC(N₁), and provides a three base cohesive end (see FIG. 1B, cf. also Szybalski et al., 1991, supra) on the counter strand.

Alternatively, the reaction may also be performed without polynucleotide kinase when PCR products have been generated with phosphorylated primers and a phosphorylated adapter oligonucleotide is used. A further alternative to the use of the adapter oligonucleotide is performing PCR following the methods described in U.S. Pat. No. 6,261,797, thereby equipping the PCR product with the combinatorial site and the recognition site for the type IIS restriction endonuclease directly. In the latter case, polynucleotide kinase and the adapter oligonucleotide may be omitted from the reaction mixture. In this connection, it is not noted that the adapter molecule does not necessarily need to form a hairpin as shown in FIG. 1B. Without wishing to be bound by theory, dimerization of 2 adapter oligonucleotide molecules is also possible and lead to the same desired result to equip the nucleic acid of interest with the type 2 IIS restriction endonuclease recognition site and ultimately the predetermined cohesive ends.

Alternatively to ligating an adapter oligonucleotide to both ends of the PCR product, the PCR product may be inserted into linearized plasmid DNA that provides the required SapI or LguI recognition sequences. The blunt ends in the adapter plasmid to ligate the PCR product comprising the nucleic acid of interest can be e.g. provided by providing an adapter plasmid comprising the following sequence:

-(N)ₓGCTCTTCG↓CGAAGAGC(N)ₓ-
-(N)ₓCGAGAAGC↓GCTTCTCG(N)ₓ-

precut with the type IIP restriction endonuclease NruI (underlined) so that after ligation, LguI or SapI (SapI and LguI are isoschizomers) cleaves in the predetermined combinatorial site (SapI/LguI recognition site is in italics) or by providing an adapter plasmid comprising the following sequence:

-(N)ₓGCTCTTCN↓(N)₅GACTC(N)₆GAGTC(N)₅↓NGAAGAGC(N)ₓ-
-(N)ₓCGAGAAGN↓(N)₅CTGAG(N)₆CTCAG(N)₅↓NCTTCTCG(N)ₓ-

precut with the type IIS restriction endonuclease SchI (underlined) so that after ligation LguI or SapI cleaves in the predetermined combinatorial site (SapI/LguI recognition site is in italics)

In other words, recircularisation of such cleaved plasmid through insertion of the PCR product by means of a ligation reaction and subsequent cleavage with SapI or LguI will equally generate the required cohesive ends at the nucleic acid molecule as shown in (FIG. 1B).

When a PCR product with attached adapter oligonucleotide is cleaved and then is ligated with a cleaved Entry vector that provides complementary cohesive ends, a Donor vector is created which is devoid of any of those type IIS restriction endonuclease recognition sequences that are used for cloning due to the initial positioning of the recognition sequences (see FIG. 1C). Therefore, said Donor vector cannot be re-cut at the combinatorial sites and accumulates during the reaction. In this regard, it is noted that the Entry vector and, for example the adapter oligonucleotide (that provides the recognition site for the type IIS restriction endonuclease to the nucleic acid of interest) do not have to comprise a recognition site for the same type II restrictions endonuclease but it is sufficient that by means of the treatment with the two restrictions endonucleases compatible/complementary cohesive ends are formed (cf. FIG. 6 which depicts essentially the same reaction as FIG. 2 with the only difference that Esp3I in the Acceptor vector has been replaced by BsaI and the mixture for the transfer reaction includes additionally BsaI).

An illustrative example for an Entry Vector providing the combinatorial sites “AATG” and “GGGA” defined by convergent Esp3I sites as shown in this FIG. 1 is pENTRY-IBA20. pENTRY-IBA20 carries the colE1 origin of replication and a kanamycin resistance gene as selectable marker and is further defined by SEQ ID NO: 22.

FIG. 2

FIG. 2 describes an example of a method to create a Destination vector by transferring a nucleic acid molecule of interest (=DNA molecule) from a Donor vector into an Acceptor vector. The nucleic acid molecule is arranged in the Donor vector in between 2 recognition sites for a type IIS restriction endonuclease so that it can be excised from said Donor vector via said type IIS restriction endonuclease. Said recognition sequences are preferably convergent so that they will be cut off from the nucleic acid molecule and remain in the (unused) vector fragment after cleavage with the corresponding type IIS restriction endonuclease. In the present illustrative example, said recognition sites are represented by recognition sites that are recognized by Esp3I (FIG. 2A). The recognition site of Esp3I is

| | | |
|---|---|---|
| 5′-CGTCTC(N₁)↓ | and/or | ↓(N₅)GAGACG-3′ |
| 3′-GCAGAG(N₅)↓ | | ↓(N₁)CTCTGC-5′ |

meaning the cleavage site is located after the first nucleotide downstream the 3′-end of the recognition site 5′-CGTCTC(N₁) and provides a four base cohesive end (see FIG. 2B or also cf. Szybalski et al., 1991, supra).

As Esp3I excises the nucleic acid molecule with cohesive ends that are compatible to cohesive ends that are generated by type IIS restriction enzyme cleavage of the Acceptor vector (in this case also Esp3I), preferably by using divergently orientated recognition sites, the nucleic acid molecule can ligate with the opened Acceptor vector to form a Destination vector. As Esp3I recognition sites are positioned in the Donor vector and the Acceptor vector so that they are absent in the Destination vector, digest and ligation can be performed simultaneously in a single reaction mixture (FIG. 2B). In this connection, it is noted that also the Donor vector and the Acceptor vector do not have to comprise a recognition site for the same type II restriction endonucleases but it is sufficient that by means of the treatment with the two restriction endonucleases compatible/complementary cohesive ends are formed (cf. FIG. 6). Thus, the first and the second type IIS restriction endonucleases used in the present invention can be the same restriction endonuclease or can also be different enzymes. Further, the two first type IIS restriction endonucleases used in the present invention can be the same restriction endonuclease or can also be different enzymes which is also the case for the second and third type IIS restriction endonucleases.

FIG. 3

### Direct Assembly of Multiple Nucleic Acid Molecules

The possibility to create multiple combinatorial sites for a single type IIS restriction endonuclease permits the assembly of the individual nucleic acid molecules in a pre-defined manner. Examples of useful applications for this mode of operation include the generation of artificial polycistronic operons or even the de novo synthesis of plasmid vectors from individual nucleic acid molecules. Nucleic acid molecules have to be cloned dependent on the position in the final Destination vector in dedicated Donor vectors.

In the example of FIG. 3, 2 nucleic acid molecules are assembled in parallel. The nucleic acid molecule 1 to be positioned upstream is arranged in a Donor vector 1 that has a 5′ combinatorial site (AATG) compatible with the 5′ combinatorial site of the Acceptor vector (TTAC) and a 3′ combinatorial site (AAAA) compatible to the 5′ combinatorial site of the Donor vector 2 (TTTT) containing the nucleic acid molecule to be positioned downstream. The nucleic acid molecule 2 to be positioned downstream in the Destination vector is present in a Donor vector 2 that has a 5′ combinatorial site (TTTT) compatible with the 3′ combinatorial site of the Donor vector 1 (AAAA) and a 3′ combinatorial site (CCCT) which is compatible with the 3′ combinatorial site (GGGA) of the Acceptor vector (see FIGS. 3A and 3B). Each of the Donor vectors comprises two convergent Esp3I recognition sites inbetween which the nucleic acid molecule 1 and nucleic acid molecule 2, respectively, are located. Both nucleic acid molecules present in Donor vectors are assembled in a directed manner into a Destination vector shown in FIG. 3B by means of a single reaction mixture (a one pot reaction) containing among other substances Donor vector 1, Donor vector 2, Acceptor vector, type IIS restriction endonuclease Esp3I (the latter to create the cohesive ends at the combinatorial sites), and ligase.

FIG. 4

### Sequential Assembly of Multiple Nucleic Acid Molecules in Entry Vectors

Entry vectors of the present invention also allow the sequential assembly of functional units composed of several individual nucleic acid molecules. Different divergent type IIS restriction endonuclease recognition sites are alternately used for each assembly step. They can be located up- or downstream of individual nucleic acid molecules. The divergent recognition site(s) used for insertion of a first nucleic acid molecule are eliminated and the divergent recognition sites required for insertion of a second nucleic acid molecule are co-transferred with the first nucleic acid molecule (A). In the Example illustrated in FIG. 4B, the different starting point Entry vectors carry different antibiotic resistance genes (either an ampicillin (Entry vector 4 used as donor vector) or a kanamycin resistance gene (Entry vector 3 used as acceptor vector)) so that the desired ligation product (Entry vector 5) can be selected from Entry vector 4 used as donor vector. Discrimination between Entry vector 3 and Entry vector 5 can be achieved by the transfer of a marker gene like lac P/Zα from Entry vector 4 into Entry vector 5 or by replacing a marker gene like lac P/Zα already present in Entry vector 5 prior to the transfer reaction.

### Substitution of Nucleic Acid Molecules

A nucleic acid molecule which is flanked on both sides by divergent oriented type IIS restriction endonuclease recognition sites can be in a further step replaced by another nucleic acid molecule (FIG. 4B).

If, e.g., nucleic acid molecule 3 in FIG. 4B represents a gene encoding a marker protein, bacterial clones that carry such Entry vector may be distinguished from bacterial clones carrying an Entry vector where said nucleic acid molecule had been substituted by another nucleic acid molecule carrying no or another marker protein.

Directionality by Changing Selectable Marker and Marker Gene from Step to Step

Cloning using a method of the invention is straightforward by using vectors with different resistance markers and wherein one of both vectors carries a nucleic acid molecule encoding a marker protein.

For example, the nucleic acid fragment designated as (N)ₓ of Entry vector 1 in FIG. 4A represents a marker gene encoding e.g. the green fluorescent protein (GFP) under the control of a constitutive promoter and Entry vector 1 further carries an ampicillin resistance gene as selectable marker. Entry vector 2 shown in FIG. 4A carries no GFP encoding gene but a kanamycin resistance gene as selectable marker. Then the desired reaction product of the reaction mixture (after incubation with Esp3I and ligase) indicated in FIG. 4A is an Entry vector 3 carrying said GFP gene and a kanamycin resistance gene. When E. coli is transformed by said reaction mixture and such transformed cells are plated on culturing plates containing kanamycin for selection, then only those cells carrying Entry vector 2 or Entry vector 3 are able to grow. Colonies harbouring Entry vector 2 are white while cells carrying Entry vector 3 should exhibit green fluorescence. Thus, such a strategy enables direct selection of E. coli cells harbouring the desired vector without the need for analyzing individual clones by further methods.

Further, when nucleic acid molecule 3 from Entry vector 4 in FIG. 4B encodes the lac P/Zα gene (alpha peptide of beta-galactosidase under control of a constitutive promoter), for example, and also carries an ampicillin resistance gene and when E. coli carrying the lacZΔM15 mutation is transformed with the vectors of the reaction mixture indicated in FIG. 4.B and selected for kanamycin resistance, the E. coli colonies harbouring the desired Entry vector 5 will develop a blue colour on X-gal containing medium while those colonies with Entry vector 3 will exhibit green fluorescence. E. coli harbouring Entry vector 4 will not grow on kanamycin plates. Thus, cells carrying the desired plasmid may be directly isolated without the need for additional analysis steps.

Summarizing, using e.g. coloured or colour developing marker genes and vectors with different selectable markers enables the straightforward development of Entry vectors carrying a multitude of nucleic acid molecules. The same strategy is possible for the straightforward transfer of nucleic acid molecules from Donor vectors into Acceptor vectors by using Acceptor vectors carrying a marker gene between the divergent type IIS recognition sites. Said marker gene is replaced by the nucleic acid molecule from the Donor vector upon creation of the Destination vector and colonies lacking the marker gene are isolated.

Circularity of the vectors is not indicated in this FIG. 4 for the sake of clarity. In addition the sequences of the relevant parts are indicated only.

FIG. 5

### Use of the Methods of the Invention for Site-Directed Mutagenesis

This figure illustrates how a single base pair A/T occurring in the target nucleic acid molecule is substituted by a G/C base pair during cloning of the target nucleic acid molecule into the Entry vector for creating a Donor vector.

The A/T pair to be replaced by the G/C pair is underlined and indicated in italics in FIG. 5A, whereas the G/C pair is underlined and depicted in bold in FIG. 5A. For this purpose two PCR reactions are carried out in parallel as illustrated in FIG. 5A. In a first PCR reaction primer 1 (forward primer) carrying a combinatorial site and primer 2 (reverse primer) carrying a C for introducing the desired mutation are employed resulting in PCR product 1 that carries the desired mutation at the 3′-end of the PCR product (the NA molecule is depicted in FIG. 5A in the conventional 5′-3′ direction). In the second PCR reaction the primer 3 that introduces the desired G in the coding strand of the target nucleic acid is used as forward primer and primer 4 that introduces the combinatorial site “CCC” at the 3′-terminus of the target nucleic acid is used as reverse primer. As shown in FIG. 5B, the two PCR products are then reacted with an adapter oligonucleotide that provides for the recognition site of the type IIS restriction endonuclease SapI (or LguI) (cf. also FIG. 1 in this regard) in the presence of ligase, polynucleotide kinase and ATP (the latter if unphosphorylated oligonucleotides are used). By so doing, the adapter oligonucleotide provides an extension for the two PCR products that carry the SapI (or LguI) recognition sites and at the same time allow for the later insertion of the mutated nucleic acid molecule into the desired functional/regulatory context of being placed in the reading frame of the ATG start codon. Similar to the directed assembly as shown in FIG. 3, incubation of these two modified PCR products with SapI as illustrated in FIG. 5C results in the PCR product of amplification reaction 1 to have a 5′ combinatorial site compatible with the 5′ combinatorial site of a respective Acceptor vector that comprises two divergent SapI recognition sites and a 3′ combinatorial site compatible to the 3′ combinatorial site of the PCR fragment of amplification reaction 2. Accordingly, the PCR product of the second amplification reaction has a 5′ combinatorial site compatible with the 3′ combinatorial site of PCR product of the first amplification reaction and a 3′ combinatorial site which is compatible with the 3′ combinatorial site of a respective Acceptor vector.

Such a procedure is of course not limited to the introduction of a single base pair substitution but also multiple substitutions, deletions and additions of sequences as well as combinations of said alterations may be similarly made using appropriately designed primers. Such technology is e.g. useful for the elimination or integration of restriction sites into a nucleic acid molecule or for codon optimization or for exchange of amino acids if a protein is encoded.

FIG. 6

FIG. 6 depicts the same transfer reaction as depicted in FIG. 2 with the difference that 2 different type IIS restriction endonucleases, Esp3I in the Donor vector (convergently oriented recognition sites) and BsaI or Eco31I (the isoschizomer of BsaI) in the Acceptor vector (divergently oriented recognition sites) are used. It is obvious for the person skilled in the art that also different type IIS restriction endonuclease recognition sites may in principle be used in e.g. the Donor vector to form the convergently oriented recognition sites or in e.g. the Acceptor vector to form the divergently oriented recognition sites, as alternative proceedings to get the same result. Essentially, all type IIS restriction endonucleases may be combined in such a reaction as long as they cut the same type of cohesive end, e.g. a 5′ overhang of 4 arbitrary bases (like Eco31I or BsaI or BveI or Esp3I or AarI or BpiI or BveI and the like) or a 5′ overhang of 3 arbitrary bases (like SapI or LguI or Eam1104I and the like) or a 3′ overhang of 2 arbitrary bases (like Eco57I or Eco57MI or GsuI or TsoI and the like) or a 3′ overhang of 1 arbitrary base (like BfuI or BfiI or HphI and the like) and the like, and as long as the sequences of the cohesive ends are compatible and as long as not too many further recognition sites occur in the used nucleic acids. Mixed reactions with “special” type IIS restriction endonucleases (cf. FIG. 7) are possible as well. For example, the “normal” type IIS restriction endonucleases generating a 3′ overhang of 2 arbitrary bases (like Eco57I or Eco57MI or GsuI or TsoI and the like, see above) could be used together with the “special” type IIS restriction endonucleases (like AlfI or BdaI and the like) generating also 3′ overhangs of 2 arbitrary bases.

FIG. 7

FIG. 7 depicts a similar transfer reaction as depicted in FIG. 2 with the difference that a “special” type IIS restriction endonuclease is used. Said “special” type, is illustrated in the example of shown in FIG. 7 by the type IIS restriction enzyme TstI which has the following recognition site

5′-CAC(N₆)TCC-3′
3′-GTC(N₆)AGG-5′

This “special” type restriction endonuclease cuts in both directions relative to the recognition site (for example TstI cuts 8 bases upstream from the 5′-end of the recognition site and 7 bases downstream from the 3′-end of the recognition site as shown in FIG. 7) and, therefore, cutting on behalf of one recognition site only may yield the same result as cutting on behalf of 2 divergently oriented recognition sites of “normal” type IIS restriction endonucleases. This is the reason why Acceptor vectors may be adequately opened by using only one recognition site of said “special” type IIS restriction endonuclease. Using said “special” type IIS restriction endonucleases may have a further advantage with general impact for all the nucleic acid transfer reactions described in the present invention: If adequate “special” type IIS restriction endonuclease are provided in Entry, Donor, and Acceptor vectors so that the melting temperature of the by-product (cf. FIG. 7A) is below the temperature at which the transfer reaction has to be performed, then said by-product will melt as soon as generated and be excluded from the reaction. Thereby, any back reaction is prevented and the reaction is driven towards formation of the Destination vector of this example. It is obvious to the scientist skilled in the art that said advantage of using such “special” type IIS restriction endonuclease to drive the reaction towards the end product can apply for all other nucleic transfer reactions of the invention as well.

FIG. 8

Instead of using the helper plasmid as donor plasmid for transferring the nucleic acid molecule into an Entry vector to create a Donor vector the helper plasmid may be designed as generic Entry vector for direct uptake of a nucleic acid molecule to generate a Donor vector or a Donor vector' (see FIG. 8) which is suitable to transfer nucleic acid molecules into Acceptor and/or other Entry vectors.

In a first step, the desired combinatorial site(s) (e.g. the combinatorial site present in a multitude of Acceptor vectors such as AATG or GGGA in 5′- and 3′-position, respectively) is attached to the nucleic acid molecule of interest (FIG. 8A). This can be achieved by performing an amplification reaction such as PCR. Preferentially, a proof reading DNA polymerase is used for PCR because such polymerases generate PCR products with blunt ends while normal Taq polymerase adds nucleotides to generate 3′ overhangs.

Further, an Entry vector containing divergent type IIS restriction endonuclease recognition sites of a type IIS restriction endonuclease generating blunt ends (illustrated by SchI in the example of FIG. 8) or any other blunt end generating restriction enzyme (for example, a type IIP restriction endonuclease) that is able to open said Entry vector with blunt ends at defined positions towards the convergent type IIS restriction endonuclease recognition sites (=Esp3I in this FIG. 8B) is provided (FIG. 8B). Said defined position assures that—after insertion of the e.g. PCR product—the resulting Donor vector is cleaved within the combinatorial sites after cleavage with the type IIS restriction endonuclease associated with the convergent type IIS recognition sites. Said Entry vector is opened by said blunt end generating restriction enzyme and the opened Entry vector is ligated with the isolated PCR product. The reaction will generate a Donor vector or a Donor vector' as no predefined orientation or is given by blunt ends. However, the same nucleic acid molecule will be generated upon cleavage with Esp3I of the present example—irrespective whether Donor vector or Donor vector' has been cleaved—thereby providing in each case the necessary cohesive ends as e.g. needed for the transfer reaction described in FIG. 2. When the combinatorial site is only partially attached to the nucleic acid molecule via e.g. PCR, then the Donor vector will differ from Donor vector' and one of both will not be suitable for the subsequent transfer reactions. One advantage of using a blunt end insertion of the nucleic acid molecule of interest into the helper plasmid or Entry vector (both is possible) is that no trimming of the terminal ends of the nucleic acid molecule of interest is necessary, thereby circumventing the problem of potential internal recognition site for the restriction enzyme used for trimming.

The present embodiment is simple, universal and straightforward to generate a Donor vector. An example for an Entry vector providing convergent Esp3I restriction enzyme recognition sites for gene transfer into Acceptor vectors (cf. FIG. 11) is pENTRY-IBA10. pENTRY-IBA10 carries the colE1 origin of replication and a kanamycin resistance gene as selectable marker and is further defined by SEQ ID NO: 23.

FIG. 9

Use of the Methods of the Invention to Fuse Two or More Nucleic Acid Molecules Present in Donor Vectors Through Transfer into Special Entry Vectors for Upstream and Downstream Fusion and Re-Introduction into the Initial Entry Vector

The methods of the invention allow bringing a nucleic acid molecule from a Donor vector into an Acceptor vector by a facile one-step subcloning procedure. A variety of pre-made different Acceptor vectors providing different genetic surroundings, e.g. to bring different promoters or purification tags into operative linkage with the nucleic acid molecule of interest, allows for the systematic screening of the optimal tool combination for efficient expression and purification of a nucleic acid molecule when this constitutes a protein encoding gene for example. When such a standardized cloning system is in use, a library of Donor vectors with cloned nucleic acid molecules of interest (genes) flanked with the identical combinatorial sites will accumulate. In some cases, it might be interesting to bring two nucleic acid molecules already present in different Donor vectors into operative linkage. Examples are, without limitation, to generate a fusion protein from two or more genes or to express different nucleic acid molecules from one promoter as polycistronic operon or to express different nucleic acid molecules from a single expression vector under control of independent promoters.

A further attractive aspect of a simple tool to generate fusions is the following. For protein expression, for example, a series of Acceptor vectors has to be provided for the systematic screen of an optimal expression host/purification tag combination which means that a separate Acceptor vector has to be constructed for each promoter/tag combination wherein each tag may be placed N- or C-terminally to the gene of interest or in conjunction with other tags in different combinations. Thus, the number of Acceptor vectors to be provided grows exponentially with the number of tags and each time when a new tag is developed many Acceptor vectors have to be constructed to make such new tag available to users of the subcloning system of the invention. To reduce here time and cost it is straightforward to provide such new tag precloned in a Donor vector for upstream fusion and in a Donor vector for downstream fusion. With these 2 vectors a user of the cloning system of the invention can easily combine its gene of interest with the new tag, both N-and C-terminally, and express it in different hosts (and in combination with different other tags) by using the already existing Acceptor vectors carrying tags and different promoters for expression in different hosts.

The strategy for fusing two nucleic acid molecules is the following:

In a first step, nucleic acid molecule 1 cloned in a Donor vector, e.g. generated via the methods of the invention (FIG. 1), intended to be fused upstream to a nucleic acid molecule 2, also cloned in a Donor vector, e.g. also generated via the methods of the invention (FIG. 1), is transferred by a one-step reaction of the invention into an Entry vector for upstream fusion (FIG. 9A) and nucleic acid molecule 2 is transferred in parallel by a similar reaction into an Entry vector for downstream fusion (FIG. 9B).

In a second step, the generated Donor vector for upstream fusion of nucleic acid molecule 1 and Donor vector for downstream fusion of nucleic acid molecule 2 are reacted by a further one-step reaction of the invention with an Entry vector (cf. FIG. 1C) to generate a Donor vector containing nucleic acid molecule 1 fused to nucleic acid molecule 2 via a linker sequence denoted (N)ₓ (FIG. 9C). Such assembled nucleic acid molecules 1 and 2 in a new Donor vector carry now upstream and downstream combinatorial sites so that the assembly may be transferred into the pre-made Acceptor vectors providing the different genetic surroundings, e.g. tools for expression and purification of the assembled nucleic acid molecules 1 and 2.

The sequence (N)ₓ provided by the Entry vector for upstream fusion determines the way in which both nucleic acid molecules are fused. If for example both nucleic acid molecules are genes encoding proteins and (N)ₓ stands for the nucleic acid sequence GC TAA CGA GGG CAA AA (containing a stop codon for nucleic acid molecule 1 (“TAA”, underlined) followed by a bacterial ribosomal binding site (Shine Dalgarno site), then nucleic acid molecule 1 may be expressed together with nucleic acid molecule 2 as separate proteins via this synthetic dicistronic operon after having transferred the fusion of nucleic acid molecules 1 and 2 present in a Donor vector (and generated as shown in FIG. 9C) into an Acceptor vector providing a bacterial promoter and transforming a bacterial host like E. coli with the resulting Destination vector.

Likewise, a direct fusion protein may be generated if nucleic acid molecules 1 and 2 are fused using an Entry vector for upstream fusion carrying a single nucleic acid base, e.g. a cytosine “C”, at the site marked with (N)ₓ. In this case, a fusion protein may be generated consisting of the protein encoded by nucleic acid molecule 1 and the protein encoded by nucleic acid molecule 2, both fused by a linker consisting of the amino acid doublet Gly-Thr. Of course, also longer sequences may be inserted to generate fusion proteins with elongated linkers as long as the insert (N)ₓ connects both nucleic acid molecules in the same reading frame and contains no stop codon in such reading frame.

An Entry vector for upstream fusion with (N)ₓ representing terminator and promoter or polyA signal and promoter may be useful for expression of 2 nucleic acid molecules under control of different promoters in bacteria or eukaryotic cells, respectively. Further, tags may be provided already cloned in a Donor vector for upstream or downstream fusion for direct N- or C-terminal fusion with a nucleic acid molecule.

It shall be noted that higher order fusions can easily be performed by repeating this procedure with already fused nucleic acid molecules. In case of higher order fusions, also combinations of the linking elements may be created to generate, e.g., without limitation, a synthetic operon where the upstream gene carries an affinity tag (using an Entry fusion vector as shown by example 6 in FIG. 9D) and the subsequent carries no tag (using an Entry fusion vector as shown by example 1 in FIG. 9D), simply by using an Entry fusion vector carrying the appropriate sequence N(x) at the dedicated step of fusion (cf FIG. 9D which enumerates some of the possible elements encoded by N_{X}). Also Entry vectors for fusion carrying random sequences at N_{X} may be used for optimization of linking elements which may be, e.g., amino acid linkers or Shine Dalgarno sequences in a certain context.

To reduce the number of subcloning steps of the invention in case of generation of higher order fusions, special Entry vectors for upstream and downstream fusion carrying a kanamycin resistance gene (if in context of the example of FIG. 9) and divergent LguI recognition sites for uptake of the fusion product can be used instead of the initial Entry vector for gene assembly from FIG. 1C. Such vectors may provide convergent Esp3I exit sites (if in context of the example of FIG. 9) and a region (N)ₓ (if in context of the example of FIG. 9) and are designed in a way that they provide compatible combinatorial sites for the fusion of cloned nucleic acid molecules and integration of the fusion product into an Entry fusion vector with ampicillin resistance of FIG. 9A or FIG. 9B or directly into an Acceptor vector carrying an ampicillin resistance gene and e.g. promoters and/or tags for gene expression. Such a second series of fusion vectors, having another resistance gene and inverted convergent and divergent type IIS restriction enzyme recognition sites, allows the rapid assembly of higher order fusions of nucleic acid molecules by shuttle reactions with Entry vectors for fusion of FIG. 9 (see also FIG. 10).

It should be noted also that higher order fusions with different linking elements ((N)ₓ) may be generated easily by using the appropriate Entry fusion vectors for fusion at the dedicated step of the assembly.

It should be noted also that a similar strategy with a different arrangement of the elements can be used for the same purpose of making fusions of nucleic acid molecules. For example, but not limited thereto, the linker element N(x) can also be integrated into the Entry vector for downstream fusion or other type IIS restriction endonuclease recognition sites than Esp3I and LguI can be used. The principal element for a cassette system is that the nucleic acid molecule is inserted into the Entry vector for gene fusion with a first typeIIS restriction enzyme using certain combinatorial sites and can be cut out with a second typeIIS restriction enzyme using at least one other combinatorial site that is positioned in a way to fuse a sequence Nx to the nucleic acid molecule and that is compatible with a combinatorial site that is present in the other Entry vector for gene fusion.

Likewise, an Entry vector for upstream fusion can also be designed—by a simple shift of the upstream Lgu I recognition site for excision relative to the upstream Esp3I recognition site for insertion so that the combinatorial sites ATG and AATG are separated by a region N(y) and not overlapping as in the current example—for fusion of the linker element N(y) upstream to the GOI, which would be for example useful for the direct fusion of individual GOIs with different affinity tags or other N-terminal fusion partners, but also for the generation of co-expression plasmids, which allow differential induction of individual genes or groups thereof under the control of different promotors.

Due to the high efficiency of the methods of the invention for subcloning nucleic acid molecules (see also experimental example 5), the methods of the invention may also be very useful for e.g. the fusion or handling of random libraries where efficiency during subcloning is crucial to preserve library diversity.

The fusion technology of FIG. 9 may for example be useful if random libraries have to be combined as it may for example be the case during the engineering of recombinant antibody fragment light and heavy chains. A further example for the utility of the fusion technology is the combination of different alleles of MHCII molecules with different antigenic peptides. MHCII molecules are composed of an alpha and beta chain and of an antigenic peptide which each could be seen as a module (nucleic acid molecule). Many different alleles are known for alpha and beta chains and also a high variety exists for antigenic peptides. MHCII together with the antigenic peptide may be recombinantly produced as single chain molecules. Thus a very useful application of the present invention is to clone the different alpha chains, beta chains and antigenic peptides in separate Donor Vectors so that the cloning of any combination may be quickly achieved by the fusion strategy outlined in this FIG. 9 and in FIG. 10.

FIG. 10

### Schematic Overview and Workflow of a Generic Subcloning System Enabled by the Methods of the Invention

A) Step 1: Donor Vector Generation (cf. FIG. 1 or 8)

In a first step, the target nucleic acid, also referred to as gene of interest (GOI) is equipped at both ends with combinatorial sites (of for example 4 bases) by PCR and is inserted into an Entry Vector by a simple one-tube reaction. The opened Entry Vector contributes the recognition sites of the type IIS restriction endonuclease and brings them into operative linkage with the combinatorial sites for the highly specific gene transfer process from Step 2.

Step 2: Destination Vector Generation (cf. FIG. 2)

After sequence confirmation, the resulting Donor Vector is the origin for exerting the option of the highly parallel subcloning of GOI by a second simple one-tube reaction via the combinatorial sites into a multitude of Acceptor Vectors, each providing a different genetic surrounding like host specific promoters and different purification tags. The resulting Destination Vectors are then transformed into the corresponding host cells for further experiments.

B) It may also be of interest to fuse two genes present already cloned and sequenced in Donor Vectors via the methods of the invention and then transfer the fused genes into an Acceptor Vector. The presented strategy (cf. also FIG. 9) uses 2 special Entry Vectors, one for positioning the inserted gene upstream and one for positioning the inserted gene downstream in the fusion gene construct. In the present example, the design of the typeIIS restriction enzyme recognition sites is such that the Entry Vector for upstream fusion provides a sequence stretch N(x) that constitutes the linker between the upstream gene (GOI1 and the present example) and the downstream gene (GOI2 in the present example) in the resulting fusion. There are, however, also other possibilities how the linker N(x) may be provided, e.g. by the Entry Vector for downstream fusion. The linker N(x) determines the function by which the 2 genes are brought into operative linkage. Examples are given in FIG. 9D.

C) Higher order fusions (also with different linkers N(x) when using the appropriate Entry Vectors for upstream fusion at the dedicated step) may be performed by repeating the reactions from FIG. 10B. If, for example, 4 genes of interest (GOI's) are to be assembled then GOI1 and GOI2 as well as GOI3 and GOI4 can be fused as shown in FIG. 10B and the fused GOI1/GOI2 and GOI3/GOI4 in the resulting Donor vectors can be introduced again into the Entry Vector for upstream fusion and Entry Vector for downstream fusion respectively. In a further step, GOI1/GOI2 and GOI3/GOI4 are assembled into the Entry Vector to constitute a Donor Vector with GOI1/GOI2/GOI3/GOI4-fusion which can be then transferred in parallel into a multitude of separate Acceptor Vectors (FIG. 11). Such procedure to generate the fusion of 4 genes from initial Donor Vectors needs 5 sequential cloning steps of the invention. The procedure can be short cut to 3 sequential cloning steps by using special short cut Entry Vectors for upstream and downstream gene fusion and by using the strategy of FIG. 10C. The special Entry Vectors for upstream and downstream fusion differ from the analogous Entry vectors from FIGS. 9A and 9B, respectively, in a way that i) they have LguI recognition sites instead of Esp3I sites for GOI uptake (in fact, the combinatorial sites have to be dedicated for GOI assembly from Entry Vectors for upstream and downstream fusion from FIGS. 9A and 9B) and ii) Esp3I sites instead of LguI sites for cutting the insert out (GOI plus N(x) for special Entry Vector for upstream fusion) and assembling it in an Acceptor Vector and iii) they are preferably devoid of the selectable marker present in the Acceptor Vector and preferably possess another selectable marker than present in the Entry Vectors for upstream and downstream fusion from FIG. 9 to make GOI transfer reactions more efficient. In the present example, Acceptor Vectors and Entry Vectors for upstream and downstream fusion from FIG. 9 contain an ampicillin resistance gene as selectable marker while the special short cut Entry Vectors for upstream and downstream fusion contain a kanamycin resistance gene as selectable marker.

FIG. 11

### Acceptor Vector Examples

### A) Overview

The table shows a series of Acceptor Vectors which can be subdivided in 4 classes:
pASG-IBA
pPSG-IBA
pYSG-IBA
pESG-IBA

The vector pASG-IBA is for tightly regulated gene expression in E. coli via the tetracycline promoter.

The vector pPSG-IBA is for high level gene expression in E. coli via the T7 promoter.

The vector pYSG-IBA is for regulated expression in yeast via the copper inducible CUP1 promoter.

The vector pESG-IBA is high level gene expression in mammalian cells via the CMV promoter.

The label (number or wt1) of each Acceptor Vector denotes a defined expression cassette which is composed of certain elements (i.e. secretion signal (E. coli or eukaryotic cells) and/or affinity tag (STREP-tag®; His-tag; GST-tag (N-terminal positioning only); sequentially arranged tags as described in US patent application 20030083474 marketed under the name “One-STrEP-tag”, and which is identical throughout the Acceptor Vector classes except for vectors with a secretion signal. The nucleic acid sequence and the corresponding polypeptide sequence of illustrative expression cassettes (termed wt-1, 3, 5, 23, 33, 35, 43, 45, 103 and 105) is depicted in FIG. 11C (see also below).

Vectors with a secretion signal differ because signal sequences for E. coli are other than for mammalian cells. The identity and order of the elements is indicated in the table for each Acceptor Vector. Each Acceptor Vector contains a cassette with lacP/Zα flanked by divergent Esp3I restriction enzyme recognition sites for uptake of a nucleic acid molecule (gene of interest; GOD cloned into a Donor Vector (cf. FIG. 1 or FIG. 8 for the generation of a Donor Vector) and for positioning the GOI in operative linkage with the elements of the expression cassette.

### B) Description of the Backbones of the Different Acceptor Vector Classes

pASG-IBA

pASG-IBA vectors as illustrated in FIG. 11B carry the promoter/operator region from the tetA resistance gene (tetA) which allows tightly controlled gene expression. The tet repressor gene is constitutively expressed as downstream element of an artificial operon from the beta lactamase promoter controlling also expression of beta lactamase gene (AmpR) as selectable marker as upstream element of said artificial operon. Constitutive expression of tet repressor enables tight repression of the promoter until addition of the inducer anhydrotetracycline (200 μg/liter culture) to the medium. In contrast to the lac promoter, which is susceptible to catabolite repression (cAMP-level, metabolic state) and chromosomally encoded repressor molecules, the tetA promoter/operator is not coupled to any cellular regulation mechanisms. Therefore, when using the tet system, there are basically no restrictions in the choice of culture medium or E. coli expression strain. For example, glucose minimal media and even the bacterial strain XL1-Blue, which carries an episomal copy of the tetracycline resistance gene, can be used for expression. Further, an f1 on for the preparation of single stranded plasmid DNA and a ColE1 on for plasmid propagation in E. coli are included. The position of the expression cassette is downstream of tetA and indicated with 2 boxes. The nucleic acid sequence of pASG-IBA vector backbone for cytosolic expression except the expression cassette is given as SEQ ID NO: 16. The chosen expression cassette is positioned between base 3060 (“A”) and base 3061 (“G”) of SEQ ID NO: 16.

Some expression cassettes carry the ompA signal sequence for secretion of the recombinant protein into the periplasmic space which is crucial for functional expression of proteins with structural disulfide bonds. In this case, the authentic Shine Dalgarno sequence of the ompA gene is used which implicates a small nucleic acid variation in the region directly upstream of the expression cassette. The nucleic acid sequence of pASG-IBA vector backbone for periplasmic secretion (comprising an expression cassette comprising the ompA signal sequence) except expression cassette is given as SEQ ID NO: 17. The expression cassette (which can be freely chosen) is positioned between base 3039 (“A”) and base 3040 (“G”) of SEQ ID NO: 17.

pPSG-IBA

pPSG-IBA vectors illustrated in FIG. 11B use the T7 promoter for high-level transcription of the gene of interest. Expression of the target genes is induced by providing a source of T7 RNA polymerase in the E. coli host cell. This is accomplished by using, e.g., an E. coli host which contains a chromosomal copy of the T7 RNA polymerase gene (e.g. BL21(DE3) which has the advantage to be deficient of Ion and ompT proteases). The T7 RNA polymerase gene is under control of the lacUV5 promoter which can be induced by addition of IPTG.

The plasmid contains the constitutively expressed beta lactamase gene (AmpR) as selectable marker. Further, an f1 ori for the preparation of single stranded plasmid DNA and a ColE1 on for plasmid propagation in E. coli are included. The position of the expression cassette is downstream of T7 and indicated with 2 boxes. The nucleic acid sequence of pPSG-IBA vector backbone except expression cassette is given as SEQ ID NO: 18. The expression cassette (which can be freely chosen) is positioned between base 2679 (“A”) and base 2680 (“G”) of SEQ ID NO: 18.

pESG-IBA

pESG-IBA vectors shown in FIG. 11B are designed for high-level constitutive expression of recombinant proteins in a wide range of mammalian host cells through the human cytomegalovirus immediate-early CMV promoter (CMV). To prolong expression in transfected cells, the vector will replicate in cell lines that are latently infected with SV40 large T antigen (e.g. COS7) trough the SV40 ori. In addition, Neomycin resistance gene allows direct selection of stable cell lines. Propagation in E. coli is supported by a ColE1 on and the beta lactamase gene (AmpR) is included as selectable marker. Transcription of the expression cassette and of the Neomycin resistance gene is terminated by a polyA signal (pA). The position of the expression cassette is downstream of CMV and indicated with 2 boxes. The nucleic acid sequence of pESG-IBA vector backbone except expression cassette is given as SEQ ID NO: 19. The expression cassette (which can be freely chosen) is positioned between base 5282 (“C”) and base 5283 (“G”) of SEQ ID NO: 19.

pYSG-IBA

pYSG-IBA expression vectors illustrated in FIG. 11B are designed for high-level expression of recombinant proteins in yeast. Cloned genes are under the control of the Cu⁺⁺-inducible CUP1 promoter (CUP1) which means that expression is induced upon addition of copper sulfate. In addition, the vectors include the E. coli beta lactamase gene as selectable marker in E. coli, and the genes leu2-d (a LEU2 gene with a truncated, but functional promoter) and URA3 as selectable markers in respectively auxotrophic yeast strains. Vectors including the leu2-d marker are maintained at high copy number to provide enough gene products from the inefficient promoter for cell survival during growth selection in minimal medium lacking leucine. Propagation in E. coli is supported by a ColE1 on and the beta lactamase gene (AmpR) is included as selectable marker. Propagation in yeast is supported by the 2 micron ori. The position of the expression cassette is downstream of CUP1 and indicated with 2 boxes. The nucleic acid sequence of pYSG-IBA vector backbone except expression cassette is given as SEQ ID NO: 20. The expression cassette (which can be freely chosen) is positioned between base 7047 (“C”) and base 7048 (“G”) of SEQ ID NO: 20.

### C) Sequences of the Expression Cassettes

The nucleic acid sequence and the corresponding polypeptide sequence of illustrative expression cassettes is depicted in FIG. 11C. The illustrative expression cassettes are termed wt-1, 3, 5, 23, 33, 35, 43, 45, 103 and 105.

These illustrative expression cassettes for cytosolic expression with a defined number in its designation are identical for each of the pASG-IBA, pPSG-IBA, pESG-IBA and pYSG-IBA backbone. Furthermore, different expression cassettes for periplasmic secretion for E. coli containing the ompA signal sequence have been generated and introduced into the pASG-IBA backbone and different expression cassettes for secretion into the medium for mammalian cells containing the BM40 signal sequence have been generated and introduced into the pESG-IBA backbone. The expression cassettes comprise a lacP/Zα element for alpha complementation of lacZΔM15 E. coli strains for blue/white selection. The lacP/Zα element is flanked by divergent Esp3I restriction endonuclease recognition sites. When a GOI, flanked by convergent Esp3I restriction endonuclease recognition sites, is transferred from a Donor Vector (cf. FIG. 1 or FIG. 8) into one of the described Acceptor Vectors via the combinatorial sites “AATG” and “GGGA”, the lacP/Zα element is replaced by GOI and the corresponding E. coli clone after transformation will lead to a white colony. The sequence of the lacP/Zα element with flanking divergent Esp3I restriction endonuclease recognition sites as inserted in the expression cassettes is defined by SEQ ID NO: 21.

It is obvious for the person skilled in the art that any further backbone of an expression vector, serving also other expression hosts like insect cells, can easily be adapted to be an Acceptor vector of the invention.

### EXPERIMENTAL EXAMPLES

### Experimental Example 1

### Cloning of GFP in a Donor Vector

### Generation of the Adapter Oligonucleotide

200 μl of a solution containing the adapter oligonucleotide (5′-CGA AGA GCC GCT CGA AAT AAT ATT CGA GCG GCT CTT CG-3′) (SEQ ID NO: 26) in a concentration of 10 μM in 1×PCR buffer with enhancer (Invitrogen; Cat. no. 11495-017) was introduced in a sealed 0.5 ml reaction vessel which was then incubated for 15 min in 600 ml boiling water. After incubation, the reaction vessel in the 600 ml water bath had been transferred into a box of Styrofoam (3 cm wall thickness). The closed Styrofoam box was incubated in the cold room (+4° C.) to allow slow cooling and annealing of the adapter oligonucleotide. The annealed adapter oligonucleotide was then stored at +4° C. in the refrigerator.

### Generation of the Donor Vector Containing as Nucleic Acid Molecule a Gene Encoding GFP

GFP was amplified by PCR using thermostable proofreading Pfu polymerase (Fermentas, Cat. no. EP0502) with dedicated primers to generate a PCR product with blunt ends (SEQ ID NO: 1) that subsequently was purified using a Kit (Qiagen, Cat. no. 27106).

The purified PCR product was transferred into an Entry vector by a reaction mixture of 50 μl with the following constituents:
50 ng Entry vector (pALD(EL)2_Kan(blue) containing the lac P/Zα gene (to be replaced by GFP gene), SEQ ID NO: 2)
0.8 μg purified PCR product encoding GFP (SEQ ID NO. 1)
25 u polynucleotide kinase (Fermentas, Cat. no. EK0032)
2.5 u T4 DNA ligase (Fermentas, Cat. no. EL0013)
10 u LguI (Fermentas, Cat. no. ER1932)
0.02 μM annealed adapter oligonucleotide (SEQ ID NO: 26)
500 μM ATP (Fermentas, Cat. no. R0441)
1× buffer Tango (Fermentas, Cat. no. BY5)
   were incubated at 25° C. for 60 min. Then, 2 μl of the mixture were added to 100 μl chemically competent E. coli XL1 blue (CaCl₂ method) and incubated on ice for 10 min. After heat shock (37° C., 5 min), transformed E. coli cells were recovered by addition of 900 μl LB medium and incubation at 37° C. for 60 minutes. Then, cells were sedimented, resuspended in 100 μl and the whole was plated on LB agar containing 50 μg/ml kanamycin, 500 μM IPTG and 50 μg/ml X-Gal and incubated overnight at 37° C. The next day, 119 white colonies and 287 blue colonies appeared on the plate. 3 white colonies were picked and correct Donor vector formation (SEQ ID NO: 3) was confirmed by restriction analysis and sequencing of the relevant fragment (1 clone).

### Experimental Example 2

### Transfer of a Nucleic Acid Fragment via LguI

A nucleic acid fragment encoding a protease cleavage site (Prescission) and the lacZ alpha peptide under control of the lac promoter (lac P/Zα) was transferred from pTS-PCS(blue) (SEQ ID NO: 4) including convergently oriented LguI recognition sites and a kanamycin resistance gene as selectable marker into pALD3.1_Amp (SEQ ID NO: 5) including divergently oriented LguI recognition sites and an ampicillin resistance gene as selectable marker thereby generating pAU-7(blue) (SEQ ID NO: 6). The transfer reaction comprises incubating
500 ng pTS-PCS(blue)
50 ng pALD3.1_Amp
2 u T4 DNA ligase (Fermentas, Cat. no. EL0013)
5 u LguI (Fermentas, Cat. no. ER1932)
0.5 mM ATP (Sigma, Cat. no. A2383)
1× buffer Tango (Fermentas, Cat. no. BY5)
   in a final volume of 50 μl for 1 h at 30° C. Then, 5 μl of the mixture were gently mixed with 50 μl chemically competent E. coli DH5α (prepared according to Inoue et al., 1990, Gene 96, pp 23-28, 2*10⁷ cfu/μg pTS_Kan) and incubated on ice for 10 min. After heat shock (42° C., 10 sec), 950 μl LB medium were added and the kanamycin resistance was allowed to develop for 1 h at 37° C. Then, 50 μl of the resulting mixture were plated on LB agar containing 50 μg/ml carbenicillin, 500 μM IPTG and 50 μg/ml X-Gal. Plates were incubated overnight at 37° C. The next day, 8 white and 583 blue colonies appeared on the plate. 10 blue colonies putatively harbouring pAU-7(blue) were picked and correct vector formation was confirmed by restriction analysis and one of the plasmids was sequenced to confirm the relevant fragment.

### Experimental Example 3

### Transfer of a Nucleic Acid Fragment Via Eco31I

A nucleic acid fragment encoding the lacZ alpha peptide under control of the lac promotor (lacP/Zα) was transferred from pAU-1(blue) (SEQ ID NO: 7) including convergently oriented Eco31I recognition sites and an ampicillin resistance gene as selectable marker into pAU-wt (SEQ ID NO: 8) including divergently oriented Eco31I recognition sites and a kanamycin resistance gene as selectable marker thereby generating pTU-((blue) (SEQ ID NO: 9). The transfer reaction comprises incubating
500 ng pAU-1(blue)
50 ng pTU-wt
2 u T4 DNA ligase (Fermentas, Cat. no. EL0013)
10 u Eco31I (Fermentas, Cat. no. ER 0291)
0.5 mM ATP (Sigma, Cat. no. A2383)
1× buffer G (Fermentas, Cat. no. BG5)
   in a final volume of 50 μl for 1 h at 30° C. Then, 5 μl of the mixture was gently mixed with 50 μl chemically competent E. coli TOP10 (prepared according to Inoue et al., 1990, Gene 96, pp 23-28, 5*10⁷ cfu/μg pUC DNA) and incubated on ice for 20 min. After heat shock (42° C., 10 sec), 950 μl LB medium were added and the kanamycin resistance was allowed to develop for 1 h at 37° C. Then, 50 μl of the resulting mixture were plated on LB agar containing 50 μg/ml kanamycin, 500 μM IPTG and 50 μg/ml X-Gal. Plates were incubated overnight at 37° C. The next day, 99 white and 124 blue colonies appeared on the plate. 10 blue colonies were picked and the formation of pTAU-((blue) was confirmed by restriction analysis and sequencing of one of the plasmids.

### Experimental example 4

### Transfer of a Nucleic Acid Fragment Via Esp3I

A nucleic acid fragment encoding the β-alanine CoA-transferase gene from Clostridium propionicum in pALD2_Kan(Act) (SEQ ID NO: 10; Donor vector) under control of the tet-promoter including convergently oriented Esp3I recognition sites and a kanamycin resistance gene as selectable marker was transferred into pEx1_CHis(blue) (SEQ ID NO: 11; Acceptor vector) including divergently oriented Esp3I recognition sites and an ampicillin resistance gene as selectable marker thereby generating pEX1_CHis-Act (SEQ ID NO: 12; Destination vector). The transfer reaction comprises incubating
500 ng pALD2_Kan(Act)
100 ng pEx1_CHis
2 u T4 DNA ligase (Fermentas, Cat. no. EL0013)
10 u Esp3I (Fermentas, Cat. no. ER0452)
0.5 mM ATP (Sigma, Cat. no. A2383)
1 mM DTT (Biomol, Cat. no. 04010)
1× buffer Tango (Fermentas, Cat. no. BY5)
   in a final volume of 50 μl for 1 h at 30° C. Then, 5 μl of the mixture was gently mixed with 50 μl chemically competent E. coli TOP10 (prepared according to Inoue et al., 1990, Gene 96, pp 23-28, 5*10⁷ cfu/μg pUC DNA) and incubated on ice for 20 min. After heat shock (42° C., 30 sec), 950 μl LB medium were added and 50 μl of the resulting mixture including transformed E. coli cells were plated on LB agar containing 50 μg/ml carbenicillin and 50 μg/ml X-Gal. Plates were incubated overnight at 37° C. The next day, 566 white and 34 blue colonies appeared on the plate. 10 white colonies putatively harbouring pEX1_CHis-Act were picked and the formation of pEX1_CHis-Act was confirmed by restriction analysis and activity test after induction of the act-gene in growing cultures supplemented with 50 ng/μL anhydrotetracycline.

### Experimental Example 5

This example provides evidence for different aspects. It shows the efficiency of the method of the invention which can be exerted with i) low amounts of plasmid DNA, ii) low amounts of type IIS restriction enzyme activity and iii) with competent E. coli cells prepared according to the CaCl₂ method which is simple and cost efficient. Further, it shows that type IIS recognition sites present internally in the genes to be transferred are not even an obstacle of performing the one-step subcloning reaction of the invention with the corresponding type IIS restriction endonuclease. In addition, this example illustrates that a working ratio between type IIS restriction endonuclease to ligase of 1:2 is shown to be suitable for one-step subcloning of such nucleic acid fragments. This example this provides further evidence, that also the assembly of multiple nucleic acid molecules can be performed efficiently in a single reaction of the invention as the transfer of nucleic acid molecules with internal recognition sites also causes the need for directional arrangement of several DNA fragments (in case of 2 internal restriction sites, four DNA fragments have to arrange in a directed manner). This is, therefore, evidence for the practicability for reactions as shown in FIGS. 3, 5, 9 and 10. Further, this example provides evidence that efficiency of the subcloning procedure of the invention is practically not influenced by the length of the transferred nucleic acid molecule but independent from the length of the nucleic acid molecule of interest.

### Materials

In a first step, nine different Donor vectors have been constructed.

The first series of 3 vectors contains the eGFP gene (714 bases in length when considered without start and stop codon; base 103 up to base 816 of SEQ ID NO:3) as nucleic molecule wherein i) one vector variant contains the eGFP gene without an internal Esp3I restriction endonuclease recognition site (SEQ ID NO: 3) and wherein ii) a further vector variant contains the eGFP gene with one internal Esp3I restriction endonuclease recognition site (SEQ ID NO: 3 with the substitution CA at position 669) and wherein iii) a last vector variant contains the eGFP gene with two internal Esp3I restriction endonuclease recognition sites (SEQ ID NO: 3 with the substitutions C→A at position 669 and G→C at position 189).

The second series of 3 vectors contains the alkaline phosphatase (phoA) gene (1409 bases in length when considered without start and stop codon; base 103 up to base 1512 of SEQ ID NO:13) as nucleic molecule wherein i) one vector variant contains the phoA gene without an internal Esp3I restriction endonuclease recognition site (SEQ ID NO: 13) and wherein ii) a further vector variant contains the phoA gene with one internal Esp3I restriction endonuclease recognition site (SEQ ID NO: 13 with the substitution A→G at position 1188) and wherein iii) a last vector variant contains the phoA gene with two internal Esp3I restriction endonuclease recognition sites (SEQ ID NO: 13 with the substitutions A→G at position 1188 and T→C at position 603).

The third series of 3 vectors contains the T7 RNA polymerase gene (2645 bases in length when considered without start and stop codon; base 103 up to base 2748 of SEQ ID NO:14) as nucleic molecule wherein i) one vector variant contains the T7 RNA polymerase gene without an internal Esp3I restriction endonuclease recognition site (SEQ ID NO: 14) and wherein ii) a further vector variant contains the T7 RNA polymerase gene with one internal Esp3I restriction endonuclease recognition site (SEQ ID NO: 14 with the substitution G→C at position 1386) and wherein iii) a last vector variant contains the T7 RNA polymerase gene with two internal Esp3I restriction endonuclease recognition sites (SEQ ID NO: 14 with the substitutions G→C at position 1386 and T→G at position 828).

The Acceptor vector pEx1_CStrep(blue) (SEQ ID NO: 15) was prepared. For investigating the effect of introducing the vector pre-cut with Esp3I into the subcloning reaction of the invention, the large Esp3I vector fragment was prepared as well.

Chemically competent E. coli TOP10 (3.5*10⁶ cfu, as measured by applying 100 pg pUC18 plasmid DNA to 100 μl competent cells) were prepared via the CaCl₂ method (Cohen et al., 1972, Proc. Natl. Acad. Sci. USA 69, 2110-2114).

The nine nucleic acid molecule variants, all present in Donor vectors, have been subcloned into the Acceptor vector pEx1_CStrep(blue) via the following reaction mixtures:

| | |
|---|---|
| pEx1_CStrep (blue) (pre-cut or circular) | 10 ng |
| Respective Donor vector | 50 ng |
| T4 DNA ligase (Fermentas, Cat. no. EL0013) | 2 units |
| Esp3I (Fermentas, Cat. no. ER0452) | 1 unit |
| ATP (Fermentas, Cat. no. R0441) | 500 μM |
| DTT (Fermentas, Cat. no. R0861) | 1 mM |
| Tango buffer (Fermentas, Cat. no. BY5) | 1x concentrated |

Each reaction mixture having a total volume of 50 μl was incubated for 60 minutes at 30° C. As control for cfu that could be achieved with the Acceptor vector alone, without any additives, 10 ng circular pEx1_CStrep(blue) in 50 μl water were incubated in parallel.

Then, a vial of 100 μl chemically competent E. coli TOP10 was transformed with 2 μl of the reaction mixture (corresponds to 400 pg Acceptor vector) via the same procedure as used for determining cfu's with pUC18 circular plasmid DNA.

The result was as follows:

| | pEx1_CStrep | | pEx1_CStrep | |
|---|---|---|---|---|
| | (blue), | | (blue), pre-cut | |
| | circular | | with Esp3I | |
| | blue | white | blue | white |
| Donor vector | colonies | colonies | colonies | colonies |
| eGFP, no internal Esp3I | 9 | ~3000 | 0 | ~1700 |
| eGFP, 1x internal Esp3I | 4 | ~1300 | 0 | ~1300 |
| eGFP, 2x internal Esp3I | 8 | ~1040 | 0 | 440 |
| phoA, no internal Esp3I | 86 | ~2500 | 2 | ~1000 |
| phoA, 1x internal Esp3I | 14 | ~2000 | 0 | 740 |
| phoA, 2x internal Esp3I | 107 | ~2000 | 0 | 550 |
| T7 RNA pol, no internal Esp3I | 15 | ~3000 | 0 | ~1500 |
| T7 RNA pol, 1x internal Esp3I | 19 | ~1500 | 0 | 720 |
| T7 RNA pol, 2x internal Esp3I | 23 | ~1100 | 0 | 570 |
| no Donor, control reaction | 1800 | 0 | | |

Plasmid DNA was prepared from 36 white colonies from the subcloning reaction with the Donor vector containing the T7 RNA polymerase gene with 2 internal Esp3I recognition sites and analyzed via XbaI/HindIII double restriction and Esp3I restriction. All of the produced DNA fragments from the plasmid DNA isolated from the 36 clones corresponded to the expected size thereby giving evidence that the subcloning reaction had performed accurately and reliably.

The experiment was for several Donor vectors from above reproduced by using the Acceptor vector pEx1_CHis(blue) (SEQ ID NO: 11) instead of pEx1_CStrep(blue) with similar results.

This example shows also that essentially the same amount of white colonies is obtained as could be obtained at all with the non-cleaved Acceptor vector alone thereby suggesting that almost all Acceptor vector present in the subcloning reaction is translated into Destination vector. Such efficiency is the more valuable as it could be obtained with economical use of enzyme based reagents and plasmid DNA.

### Experimental Example 6

### Use of the Fusion Technology of FIG. 9 for Generating an Expression Vector for an Dicistronic Operon

### Objective

The gene for bacterial alkaline phosphatase (BAP) should be fused with the gene for GFP via a ribosomal binding site (Shine Dalgarno site, cf example 1, FIG. 9D) for expression of both proteins from a dicistronic operon after subcloning into a suitable Acceptor Vector. From the resulting Destination vector, BAP should be secreted to the periplamic space of E. coli and GFP should be expressed in the cytosol simultaneously. This had been achieved by performing the following steps:

### Performance

A) Transfer of the gene encoding BAP from a Donor Vector (SEQ ID NO: 13) into an Entry Vector for upstream fusion, i.e. pFFrbs3a(blue) (SEQ ID NO: 24; N(x) according to example 1 of FIG. 9D) via Esp3I and AATG and GGGA combinatorial sites. The following reagents were mixed:

| | |
|---|---|
| pFFrbs3a (blue) (SEQ ID NO: 24) | 5 ng |
| Donor vector with BAP (SEQ ID NO: 13) | 25 ng |
| T4 DNA ligase (Fermentas, Cat. no. EL0335) | 1 unit |
| Esp3I (Fermentas, Cat. no. ER0452) | 0.5 units |
| ATP (Fermentas, Cat. no. R0441) | 500 μM |
| DTT (Fermentas, Cat. no. R0861) | 1 mM |
| Buffer B (Fermentas, Cat. no. BB5) | 1x concentrated |

The mixture was incubated in a volume of 25 μl for 1 hour at 30° C.

B) Transfer of the gene encoding GFP from a Donor Vector (SEQ ID NO: 3) into an Entry Vector for downstream fusion, i.e. pFFc(blue) (SEQ ID NO: 25) via Esp3I and AATG and GGGA combinatorial sites. The following reagents were mixed:

| | |
|---|---|
| pFFc (SEQ ID NO: 25) | 5 ng |
| Donor vector with GFP (SEQ ID NO: 3) | 25 ng |
| T4 DNA ligase (Fermentas, Cat. no. EL0335) | 1 unit |
| Esp3I (Fermentas, Cat. no. ER0452) | 0.5 units |
| ATP (Fermentas, Cat. no. R0441) | 500 μM |
| DTT (Fermentas, Cat. no. R0861) | 1 mM |
| Buffer B (Fermentas, Cat. no. BB5) | 1x concentrated |

The mixture was in a volume of 25 μl for 1 hour at 30° C.

C) E. coli TOP10 was transformed with 10 μl of each of the reaction mixture from A) and B) and cells were plated on LB-Agar with 100 mg/L ampicillin and 50 mg/L X-Gal. Plates were incubated at 37° C. The next day, DNA minipreparation from a white colony was performed for each reaction and integration of the GFP and BAP genes into pFFc(blue) and pFFrbs3a(blue), respectively, was verified by restriction analysis. The resulting vectors were called pFFc-GFP and pFFrbs3a-BAP respectively.

D) One-step fusion of BAP gene with GFP gene in pENTRY-IBA20. The following reagents were mixed:

| | |
|---|---|
| Donor Vector pFFc-GFP | 50 ng |
| Donor Vector pFFrbs3a-BAP | 50 ng |
| Entry Vector pENTRY-IBA20 (SEQ | 10 ng |
| ID NO: 22) | |
| T4 DNA ligase (Fermentas, Cat. no. EL0335) | 1 unit |
| Lgul (Fermentas, Cat. no. ER1932) | 1 unit |
| ATP (Fermentas, Cat. no. R0441) | 500 μM |
| Tango buffer (Fermentas, Cat. no. BY5) | 1x concentrated |

The mixture was incubated in a volume of 25 μl for 1 hour at 30° C. Then, E. coli TOP10 was transformed with 10 μl of the reaction and cells were plated on LB-Agar with 50 mg/L kanamycin and 50 mg/L X-Gal. Plates were incubated at 37° C. The next day, DNA minipreparation was performed from a white colony and integration of the GFP/BAP fusion into pENTRY-IBA20 was verified by restriction analysis. The resulting Donor vector was called pFF-GFP/BAP. It includes the gene for BAP fused upstream to the gene for GFP with a Shine Dalgarno sequence as linking element. The gene fusion is flanked with convergent Esp3I sites defining AATG and GGGA as combinatorial sites. Thus, the gene fusion (synthetic operon) could be transferred via the methods and reagents of the invention into any of the vectors listed in FIG. 11A.

E) To test whether both genes could be expressed from the artificial operon, created by using the methods and reagents of the invention, the fusion of the GFP and BAP genes was transferred from the Donor Vector pFF-GFP/BAP into the Acceptor vector pASG-IBA44 (see FIG. 11). The following reagents were mixed:

| | |
|---|---|
| pFF-GFP/BAP | 25 ng |
| pASG-IBA44 | 5 ng |
| T4 DNA ligase (Fermentas, Cat. no. EL0335) | 1 unit |
| Esp3I (Fermentas, Cat. no. ER0452) | 0.5 units |
| ATP (Fermentas, Cat. no. R0441) | 500 μM |
| DTT (Fermentas, Cat. no. R0861) | 1 mM |
| Buffer B (Fermentas, Cat. no. BB5) | 1x concentrated |

The mixture was incubated in a volume of 25 μl for 1 hour at 30° C. E. coli TOP10 was transformed with 10 μl of the reaction mixture and cells were plated on LB-Agar with 100 mg/L ampicillin and 50 mg/L X-Gal. Plates were incubated at 37° C. The next day, DNA minipreparation was performed from a white colony and the generation of the expected Destination vector was verified by restriction analysis. E. coli BL21(DE3) was transformed with the Destination Vector plasmid DNA and protein expression was performed following standard protocols available @iba-go.com. Briefly, 200 ml fresh LB medium with 100 mg/L ampicillin was inoculated with a fresh colony and protein expression was induced by the addition of 200 μg/L anhydrotetracycline after the optical density of the culture reached OD550=0.5. 3 hours after induction, cells were harvested. A small sample was saved for total cell analysis. Then, the content of the periplasmic space of the cells was released by a treatment with ice-cold buffer containing 1 mM EDTA and 500 mM sucrose and incubation on ice. The resulting spheroblasts were sedimented by centrifugation and the supernatant was saved as periplasmic extract fraction. Then the spheroblasts were resuspended in a buffer compatible with His-tag purification and lysed by sonication. Insoluble cell debris was sedimented by centrifugation and the supernatant was saved as cytosolic extract fraction. The BAP-Strep-tag fusion protein could be detected in and purified from the periplasmic extract while the GFP-His-tag fusion protein could be detected in and purified from the cytosolic extract after respective Western blot analysis and affinity purification (Data not shown). This showed that the fusion reactions have resulted in a functional expression vector (Destination Vector) and is in coincidence with the expected configuration of the functional elements in the expression cassette: -ompA-Strep-tagII-BAP-ShineDalgarno-GFP-His-tag-

### EQUIVALENTS

The foregoing written specification is considered to be sufficient to enable one skilled in the art to practice the invention. Indeed, various modifications of the above-described methods for carrying out the invention which are obvious to those skilled in the field of molecular biology or related fields are intended to be within the scope of the following claims.

What is claimed is:

1. Method of (sub)cloning at least one nucleic acid molecule of interest comprising
a) providing at least one (replicable) Entry vector into which the at least one nucleic acid molecule of interest is to be inserted, wherein the at least one Entry vector carries two recognition sites for at least one first type IIS and/or type IIS like restriction endonuclease and wherein said at least one nucleic acid molecule of interest can be excised from the at least one Entry vector at two combinatorial sites with one (same) or more (different) cohesive ends that are formed by the at least one first type IIS or type IIS like restriction endonuclease, and
b) providing an Acceptor vector, into which the at least one nucleic acid molecule of interest is transferred from the at least one Entry vector carrying the at least one nucleic acid molecule of interest, wherein said Acceptor vector comprises at least one recognition site for at least one second type IIS restriction endonuclease and/or at least one recognition sites for at least one type IIS like restriction endonuclease, and wherein said Acceptor vector provides two combinatorial sites identical to the two combinatorial sites present in the Entry vector.

2. (canceled)

3. The method of claim 1, wherein the two recognition sites of the at least first type IIS restriction endonucleases are arranged in the Entry vector in such relation to the combinatorial sites that the combinatorial sites are positioned in between said two type IIS restriction endonuclease recognition sites.

4. The method of claim 3, wherein the Entry vector further comprises one or two recognition sites of at least one third type IIS restriction endonuclease, wherein these recognition sites are arranged such in the Entry vector that they are positioned in between the two recognition sites of the at least one first type IIS and/or type IIS like restriction endonuclease.

5. The method of claim 4, wherein the Entry vector further comprises two second combinatorial sites that are associated with the one or two recognition site(s) of the third type IIS restriction endonuclease.

6. The method of claim 5, wherein the one or two recognition site(s) of the third type IIS restriction endonuclease are arranged such in the Entry vector in relation to their associated combinatorial sites that said recognition site(s) are positioned in between said associated combinatorial sites.

7. The method of claim 1, comprising, prior to inserting the nucleic acid of interest into the Entry vector, equipping the nucleic acid molecule of interest with combinatorial sites that have identical sequence with the combinatorial sites that are associated with the at least one third type IIS restriction endonuclease recognition site(s).

8. The method of claim 7, wherein the nucleic molecule of interest is equipped with said combinatorial sites that are compatible with the combinatorial sites that are associated with the at least one third type IIS restriction endonuclease recognition site(s) by means of oligonucleotide primers comprising the nucleotide sequence of said combinatorial sites.

9. The method of claim 8, wherein said oligonucleotide primers equip the nucleotide acid molecule of interest with said combinatorial sites in an amplification reaction or in a ligation reaction.

10. The method of claim 7, further comprising equipping the nucleic acid molecule of interest with cohesive ends that are compatible with the cohesive ends that are formed by the at least one third type IIS restriction endonuclease.

11-14. (canceled)

15. The method of claim 7, further comprising incubating the nucleic acid molecule of interest and the Entry vector in the presence of the at least one third type IIS restriction endonuclease and ligase, thereby inserting the nucleic acid molecule of interest into the Entry vector via the cohesive ends formed by the at least one third type IIS restriction endonuclease, thereby creating a Donor vector.

16-18. (canceled)

19. The method of any claim 15, comprising transforming a suitable host organism with the reaction mixture containing the Donor vector carrying the nucleic acid molecule of interest and identifying transformed hosts cells comprising the Donor vector carrying the nucleic acid molecule of interest.

20. (canceled)

21. The method of claim 19, wherein cleavage of the combinatorial sites of the at least one first type IIS restriction endonuclease and/or type IIS like restriction endonuclease in the Donor vector carrying the nucleic molecule of interest provides cohesive ends that are compatible with the cohesive ends of a linearized Acceptor vector.

22. (canceled)

23. The method of claim 21, wherein the two recognition sites of the first type IIS restriction endonuclease and/or type IIS like restriction endonuclease are identical to the at least one recognition sites of the second type IIS restriction endonuclease and/or type IIS like restriction endonuclease of the Acceptor vector.

24. The method of claim 21, wherein the at least one first type IIS restriction endonuclease is selected from the group consisting of Esp3I, Eco31I, BsaI, BveI, AarI, BpiI and BveI.

25. The method of claim 21, further comprising incubating the Donor vector carrying the nucleic acid molecule of interest and the Acceptor vector in the presence of the at least one first type IIS restriction endonuclease and/or type IIS like restriction endonuclease and the at least one second type IIS restriction endonuclease and/or type IIS like restriction endonuclease and ligase, thereby cleaving the Donor vector and Acceptor vector and transferring the nucleic acid molecule into the Acceptor vector (thereby generating a Destination vector).

26. The method of claim 15, wherein the Entry vector is provided to the reaction mixture either in circularized or linearized form.

27. (canceled)

28. The method of claim 25, wherein the Acceptor vector is provided to the reaction mixture either in circularized form or in linearized form.

29. (canceled)

30. The method of claim 1, wherein the cohesive ends are formed as an overhang selected from the group consisting of a nucleotide sequence of 5 bases in length, a non-palindromic nucleotide sequence with 4 bases in length, a nucleotide sequence of 3 bases in length, a non-palindromic nucleotide sequence of 2 bases in length, and a nucleotide sequence of 1 base in length.

31. The method of claim 30, wherein the nucleotide sequence of the overhang is selected from a sequence of the group consisting of GAATG, AAATG, AAAGG, GGGGA, GGGGC, GGGTC, GGGCA, TAAGC, TGCTC, CCCTC, GAGAG, ATCGG, AAGGG, GCCCT, GCCGC, ATTGA, GAAAA, CCCGC, CTCCT, AATG, GGGA, TAAG, GAAT, AAAT, AAAG, GGGG, GGGT, GGGC, TGCT, GAGA, ATCG, GCTG, GGCT, TCCT, CCCT, CCCG, TGCT, TTTT, TCTC, TCCG, CCGC, CAAA, CTCC, ATTG, GAAA, ATG, GGG, AAT, TCC, TCT, AGC, TGC, CCC, GCT, TGG, GAA, GAG, AGG, AAA, ATA, CTT, CTC, TTG, GTT, TTT, ACT, TAC, CAA, CAT, GAT, CGT, CGC, TAA, TAG, TGA, TA, TG, GG, CC, CT, GA, AG, A, G, T, C and the respective complementary sequence.

32-34. (canceled)

35. A nucleic acid cloning kit comprising in two separate parts a) in the first part a (replicable) Entry vector into which the at least one nucleic acid molecule of interest is to be inserted, wherein the at least one Entry vector carries two recognition sites for a at least one first type IIS restriction endonuclease and/or one at least one type IIS like restriction endonuclease and wherein said at least one nucleic acid molecule of interest can be excised from the at least one Entry vector at two combinatorial sites with one (same) or more (different) cohesive ends that are formed by the at least one first type IIS and/or type IIS like restriction endonuclease, and b) in the second part at least one Acceptor vector, into which the at least one nucleic acid molecule of interest can be transferred from the at least one Entry vector with inserted nucleic acid molecule (Donor vector) carrying the at least one nucleic acid molecule of interest, wherein said Acceptor vector comprises at least one recognition site for a second type IIS restriction endonuclease and/or type IIS like restriction endonuclease, and wherein said Acceptor vector provides combinatorial sites identical to the two combinatorial sites present in the Entry vector.

36-60. (canceled)

61. A method of (sub)cloning at least one nucleic acid molecule of interest from a replicable Donor vector into an Acceptor vector, said Donor vector comprising the nucleic acid molecule of interest to be transferred into the Acceptor vector, wherein said Donor vector carries two recognition sites for an at least one first type IIS or type IIS like restriction endonuclease and wherein said nucleic acid molecule of interest can be excised from the at least one Donor vector at two combinatorial sites with one (same) or more (different) cohesive ends that are formed by the at least one first type IIS or type IIS like restriction endonuclease, wherein the two recognition sites of the at least one first type IIS restriction endonuclease are arranged in the Donor vector in such relation to the combinatorial sites that said combinatorial sites are positioned in between these two type IIS restriction endonuclease recognition sites, and wherein the two combinatorial sites are identical in sequence to two combinatorial sites present in the corresponding Acceptor vector, said method comprising providing the Acceptor vector, into which the at least one nucleic acid molecule of interest is transferred from the at least one Donor vector carrying the at least one nucleic acid molecule of interest, wherein said Acceptor vector is linearized and provides overhangs of two combinatorial sites identical to the two combinatorial sites present in the Donor vector and wherein said combinatorial sites comprise a nonpalindromic nucleic acid sequence.

62-64. (canceled)
